(19) United States
(12) Patent Application Publication
(10) Pub. No.: US 2015/0134047 A1
(43) Pub. Date: May 14, 2015

(54) SAFE DRUG ELUTING STENT WITH ABSORBABLE COATING

(71) Applicant: Micell Technologies, Inc., Durham, NC (US)

(72) Inventors: James B. McClain, Raleigh, NC (US); Charles Douglas Taylor, Franklinton, NC (US)

(21) Appl. No.: 14/402,839

(22) PCT Filed: May 21, 2013
(86) PCT No.: PCT/US13/42093

§ 371 Date: Nov. 21, 2014

Related U.S. Application Data
(60) Provisional application No. 61/649,832, filed on May 21, 2012.
(60) Provisional application No. 61/716,437, filed on Oct. 19, 2012.

(51) Int. Cl. A61L 31/16; A61L 31/10; A61F 2/86

(52) U.S. Cl. CPC A61L 31/16; A61F 2/86; A61L 31/10; A61L 2420/06; A61L 2300/606; A61L 2300/404; A61L 2300/204
USPC 623/1.42; 424/423; 514/291

(57) ABSTRACT

Provided herein is a device comprising: a. stent; b. a plurality of layers on said stent framework to form said device; wherein at least one of said layers comprises a bioabsorbable polymer and at least one of said layers comprises one or more active agents; wherein at least part of the active agent is in crystalline form.

### CROSS REFERENCE

This application claims the benefit of U.S. provisional application Ser. No. 61/649,832, filed May 21, 2012, and U.S. provisional application Ser. No. 61/716,437, filed Oct. 19, 2012, which are incorporated by reference in their entirety.

This application is related to the following provisional patent applications: U.S. Provisional Application No. 61/556,742, filed Nov. 7, 2011, U.S. Provisional Application No. 61/581,057, filed Dec. 28, 2011, and U.S. Provisional Application No. 61/623,469, filed Apr. 12, 2012, the entire contents of which are incorporated herein by reference.

This application is also related to the following co-pending patent applications: U.S. application Ser. No. 12/426,198; U.S. application Ser. No. 12/751,902; and U.S. application Ser. No. 12/762,007, and U.S. application Ser. No. 13/086,335, and U.S. application Ser. No. 13/445,723, the entire contents of which are incorporated herein by reference.

### BACKGROUND OF THE INVENTION

Drug-eluting stents are used to address the drawbacks of bare stents, namely to treat restenosis and to promote healing of the vessel after opening the blockage by PCI/stenting. Some current drug eluting stents can have physical, chemical and therapeutic legacy in the vessel over time. Others may have less legacy, but are not optimized for thickness, deployment flexibility, access to difficult lesions, and minimization of vessel wall intrusion.

### SUMMARY OF THE INVENTION

The present invention relates to methods for forming stents comprising a bioabsorbable polymer and a pharmaceutical or biological agent in powder form onto a substrate.

It is desirable to have a drug-eluting stent with minimal physical, chemical and therapeutic legacy in the vessel after a proscribed period of time. This period of time is based on the effective healing of the vessel after opening the blockage by PCI/stenting (currently believed by leading clinicians to be 6-18 months).

It is also desirable to have drug-eluting stents of minimal cross-sectional thickness for (a) flexibility of deployment (b) access to small vessels and/or tortuous lesions (c) minimized intrusion into the vessel wall and blood.

Provided herein is a method comprising providing a device comprising a stent and a coating on the stent wherein the coating comprises PLGA and crystalline sirolimus and wherein following implantation into a subject no more than 25% of the struts are malapposed at 4 months, no more than 20% of the struts are malapposed at 6 months, and no more than 10% of the struts are malapposed at 8 months. Further provided herein is a method comprising providing a device comprising a stent and a coating on the stent wherein the coating comprises PLGA and crystalline sirolimus and wherein following implantation into a subject no more than 25% of the struts are malapposed at 4 months, no more than 20% of the struts are malapposed at 6 months, no more than 10% of the struts are malapposed at 8 months, and no more than 5% of the struts are malapposed at 18 months. In some embodiments, at 8 months, no more than 5% of the struts are malapposed. In some embodiments, at 18 months, no more than 2% of the struts are malapposed. In some embodiments, a lot of devices which includes the device has an average percent malapposition of stent struts of about 1% at 4 months, about 1% at 6 months, and/or about 0.5% at 8 months. In some embodiments, a lot of devices which includes the device has an average percent malapposition of stent struts of about 1% at 4 months, about 1% at 6 months, about 0.5% at 8 months, and/or about 0.1% at 18 months. In some embodiments, a lot of devices which includes the device has an average percent malapposition of stent struts of less than 1% at 4 months, less than 1% at 6 months, less than 0.5% at 8 months and/or less than 0.1% at 18 months. In some embodiments, a lot of devices which includes the device has an average percent malapposition of stent struts of less than 1% at 4 months, less than 1% at 6 months, and/or less than 0.5% at 8 months. In some embodiments, a lot of devices which includes the device has an average percent malapposition of stent struts of less than 1% at 4 months, less than 1% at 6 months, less than 0.5% at 8 months, and/or less than 0.1% at 18 months. In some embodiments, a lot of devices which includes the device has an average percent of malapposed stent struts that trends downward over time. In some embodiments, there is a homogeneous distribution of malapposed stent struts along the stent at any one or more of 4, 6, and 8 months following implantation of the device in the subject. In some embodiments, there is a homogeneous distribution of malapposed stent struts along the stent at any one or more of 4, 6, 8, and 18 months following implantation of the device in the subject. In some embodiments, there is no abnormal intraluminal tissue related with any malapposed stent struts of the device.

Provided herein is a method comprising providing a device comprising a stent and a coating on the stent wherein the coating comprises PLGA and crystalline sirolimus and wherein following implantation into a subject there is no more than 50% of the struts are uncovered at 4 months, no more than 40% of the struts are uncovered at 6 months, and no more than 15% of the struts are uncovered at 8 months. Further provided herein is a method comprising providing a device comprising a stent and a coating on the stent wherein the coating comprises PLGA and crystalline sirolimus and wherein following implantation into a subject there is no more than 50% of the struts are uncovered at 4 months, no more than 40% of the struts are uncovered at 6 months, no more than 15% of the struts are uncovered at 8 months, and no more than 10% of the struts are uncovered at 18 months. In some embodiments, at 8 months, no more than 10% of the struts are uncovered. In some embodiments, at 18 months, no more than 5% of the struts are uncovered. In some embodiments, at 18 months, no more than 1% of the struts are uncovered. In some embodiments, a lot of devices which includes the device has an average percent of uncovered stent struts of about 10% at 4 months, about 5% at 6 months, and/or about 5% at 8 months. In some embodiments, a lot of devices which includes the device has an average percent of uncovered stent struts of about 10% at 4 months, about 5% at 6 months, about 5% at 8 months, and/or about 1% at 18 months. In some embodiments, a lot of devices which includes the device has an average percent of uncovered stent struts of less than 11% at 4 months, less than 5% at 6 months, and/or less than 5% at 8 months. In some embodiments, a lot of devices which includes the device has an average percent of uncovered stent struts of less than 11% at 4 months, less than 5% at 6 months, less than 5% at 8 months, and/or less than 1% at 18 months. In some embodiments, a lot of devices which includes the device has an average percent of uncovered stent struts that trends downward over time. In some embodiments, there is a homogeneous distribution of uncovered stent struts along the stent at any one or more of 4, 6, and 8 months following implantation of the device in the subject. In some embodiments, there is a homogeneous distribution of uncovered stent struts along the stent at any one or more of 4, 6, 8, and 18 months following implantation of the device in the subject. In some embodiments, there is no abnormal intraluminal tissue related with any uncovered stent struts of the device.

Provided herein is a method comprising providing a device comprising a stent and a coating on the stent wherein the coating comprises PLGA and crystalline sirolimus and wherein following implantation into a subject at least 0.02 mm of neointimal thickness exists on the stent on average at 4 months, at least 0.02 mm of neointimal thickness exists on the stent on average at 6 months, at least 0.06 mm of neointimal thickness exists on the stent on average at 8 months. In some embodiments, a lot of devices which includes the device has an average neointimal thickness of about 0.07 mm at 4 months, about 0.1 mm at 6 months, and/or about 0.14 mm at 8 months. In some embodiments, a lot of devices which includes the device has an average neointimal thickness of at least 0.05 mm at 4 months, about 0.075 mm at 6 months, and/or about 0.1 mm at 8 months. In some embodiments, a lot of devices which includes the device has an average neointimal thickness that trends upward over time. In some embodiments, there is a statistically significant difference in the neointimal thickness between the 4 month and the 8 month time point with respect to average neointimal thickness for the lot.

In some embodiments, at most 10% of the stent struts are covered by fibrin at 4 months following implantation, at most 4% of the stent struts are covered by fibrin at 6 months following implantation, or at most 1% of the stent struts are covered by fibrin at 8 months following implantation. In some embodiments, a MACE rate of a lot of devices which includes the device is at most 5%, between 1% and 5%, less than 5%, less than 4%, between 3% and 5%, or less than 3.5%. In some embodiments, there is no target vessel revascularization and/or there is no stent thrombosis from implantation until 12 months following implantion. In some embodiments, there is no target vessel revascularization and/or there is no stent thrombosis from implantation until 18 months following implantion.

Provided herein is a method comprising providing a device comprising a stent and a coating on the stent wherein the coating comprises PLGA and crystalline sirolimus and wherein following implantation into a subject the late lumen loss is less than 0.55 mm, less than 0.50 mm, less than 0.45 mm, less than 0.40 mm, less than 0.35 mm, or less than 0.30 mm at 9 months following implantation.

Provided herein is a method comprising providing a device comprising a stent and a coating on the stent wherein the coating comprises PLGA and crystalline sirolimus and wherein following implantation into a subject the target lesion revascularization (TLR) is less than 1.5%, or less than 1.0% at 9 months following implantation.

Provided herein is a method comprising providing a device comprising a stent and a coating on the stent wherein the coating comprises PLGA and crystalline sirolimus and wherein following implantation into a subject the MACE rate is less than 6.5%, less than 6.0%, less than 5.5%, less than 5.0%, less than 4.5%, or less than 4.0% at 9 months following implantation.

Provided herein is a method comprising providing a device comprising a stent and a coating on the stent wherein the coating comprises PLGA and crystalline sirolimus and wherein following implantation into a subject endothelial function is maintained with no incidence of vasoconstriction at 9 months following implantation.

Provided herein is a device comprising a stent and a coating on the stent wherein the coating comprises PLGA and crystalline sirolimus and wherein following implantation into a subject no more than 25% of the struts are malapposed at 4 months, no more than 20% of the struts are malapposed at 6 months, and no more than 10% of the struts are malapposed at 8 months. Further provided herein is a device comprising a stent and a coating on the stent wherein the coating comprises PLGA and crystalline sirolimus and wherein following implantation into a subject no more than 25% of the struts are malapposed at 4 months, no more than 20% of the struts are malapposed at 6 months, no more than 10% of the struts are malapposed at 8 months, and no more than 5% of the struts are malapposed at 18 months. In some embodiments, at 8 months, no more than 5% of the struts are malapposed. In some embodiments, at 18 months, no more than 2% of the struts are malapposed. In some embodiments, a lot of devices which includes the device has an average percent malapposition of stent struts of about 1% at 4 months, about 1% at 6 months, and/or about 0.5% at 8 months. In some embodiments, a lot of devices which includes the device has an average percent malapposition of stent struts of about 1% at 4 months, about 1% at 6 months, about 0.5% at 8 months, and/or about 0.1% at 18 months. In some embodiments, a lot of devices which includes the device has an average percent malapposition of stent struts of less than 1% at 4 months, less than 1% at 6 months, less than 0.5% at 8 months and/or less than 0.1% at 18 months. In some embodiments, a lot of devices which includes the device has an average percent malapposition of stent struts of less than 1% at 4 months, less than 1% at 6 months, and/or less than 0.5% at 8 months. In some embodiments, a lot of devices which includes the device has an average percent malapposition of stent struts of less than 1% at 4 months, less than 1% at 6 months, less than 0.5% at 8 months, and/or less than 0.1% at 18 months. In some embodiments, a lot of devices which includes the device has an average percent of malapposed stent struts that trends downward over time. In some embodiments, there is a homogeneous distribution of malapposed stent struts along the stent at any one or more of 4, 6, and 8 months following implantation of the device in the subject. In some embodiments, there is a homogeneous distribution of malapposed stent struts along the stent at any one or more of 4, 6, 8, and 18 months following implantation of the device in the subject. In some embodiments, there is no abnormal intraluminal tissue related with any malapposed stent struts of the device.

Provided herein is a device comprising a stent and a coating on the stent wherein the coating comprises PLGA and crystalline sirolimus and wherein following implantation into a subject there is no more than 50% of the struts are uncovered at 4 months, no more than 40% of the struts are uncovered at 6 months, and no more than 15% of the struts are uncovered at 8 months. Further provided herein is a device comprising a stent and a coating on the stent wherein the coating comprises PLGA and crystalline sirolimus and wherein following implantation into a subject there is no more than 50% of the struts are uncovered at 4 months, no more than 40% of the struts are uncovered at 6 months, no more than 15% of the struts are uncovered at 8 months, and no more than 10% of the struts are uncovered at 18 months. In some embodiments, at 8 months, no more than 10% of the struts are uncovered. In some embodiments, at 18 months, no more than 5% of the struts are uncovered. In some embodiments, at 18 months, no more than 1% of the struts are uncovered. In some embodiments, a lot of devices which includes the device has an average percent of uncovered stent struts of about 10% at 4 months, about 5% at 6 months, and/or about 5% at 8 months. In some embodiments, a lot of devices which includes the device has an average percent of uncovered stent struts of about 10% at 4 months, about 5% at 6 months, about 5% at 8 months, and/or about 1% at 18 months. In some embodiments, a lot of devices which includes the device has an average percent of uncovered stent struts of less than 11% at 4 months, less than 5% at 6 months, and/or less than 5% at 8 months. In some embodiments, a lot of devices which includes the device has an average percent of uncovered stent struts of less than 11% at 4 months, less than 5% at 6 months, less than 5% at 8 months, and/or less than 1% at 18 months. In some embodiments, a lot of devices which includes the device has an average percent of uncovered stent struts that trends downward over time. In some embodiments, there is a homogeneous distribution of uncovered stent struts along the stent at any one or more of 4, 6, and 8 months following implantation of the device in the subject. In some embodiments, there is a homogeneous distribution of uncovered stent struts along the stent at any one or more of 4, 6, 8, and 18 months following implantation of the device in the subject. In some embodiments, there is no abnormal intraluminal tissue related with any uncovered stent struts of the device.

Provided herein is a device comprising a stent and a coating on the stent wherein the coating comprises PLGA and crystalline sirolimus and wherein following implantation into a subject at least 0.02 mm of neointimal thickness exists on the stent on average at 4 months, at least 0.02 mm of neointimal thickness exists on the stent on average at 6 months, at least 0.06 mm of neointimal thickness exists on the stent on average at 8 months. In some embodiments, a lot of devices which includes the device has an average neointimal thickness of about 0.07 mm at 4 months, about 0.1 mm at 6 months, and/or about 0.14 mm at 8 months. In some embodiments, a lot of devices which includes the device has an average neointimal thickness of at least 0.05 mm at 4 months, about 0.075 mm at 6 months, and/or about 0.1 mm at 8 months. In some embodiments, a lot of devices which includes the device has an average neointimal thickness that trends upward over time. In some embodiments, there is a statistically significant difference in the neointimal thickness between the 4 month and the 8 month time point with respect to average neointimal thickness for the lot.

In some embodiments, at most 10% of the stent struts are covered by fibrin at 4 months following implantation, at most 4% of the stent struts are covered by fibrin at 6 months following implantation, or at most 1% of the stent struts are covered by fibrin at 8 months following implantation. In some embodiments, a MACE rate of a lot of devices which includes the device is at most 5%, between 1% and 5%, less than 5%, less than 4%, between 3% and 5%, or less than 3.5%. In some embodiments, there is no target vessel revascularization and/or there is no stent thrombosis from implantation until 12 months following implantion. In some embodiments, there is no target vessel revascularization and/or there is no stent thrombosis from implantation until 18 months following implantion.

Provided herein is a device comprising a stent and a coating on the stent wherein the coating comprises PLGA and crystalline sirolimus and wherein following implantation into a subject the late lumen loss is less than 0.55 mm, less than 0.50 mm, less than 0.45 mm, less than 0.40 mm, less than 0.35 mm, or less than 0.30 mm at 9 months following implantation.

Provided herein is a device comprising a stent and a coating on the stent wherein the coating comprises PLGA and crystalline sirolimus and wherein following implantation into a subject the target lesion revascularization (TLR) is less than 1.5%, or less than 1.0% at 9 months following implantation.

Provided herein is a device comprising a stent and a coating on the stent wherein the coating comprises PLGA and crystalline sirolimus and wherein following implantation into a subject the MACE rate is less than 6.5%, less than 6.0%, less than 5.5%, less than 5.0%, less than 4.5%, or less than 4.0% at 9 months following implantation.

Provided herein is a device comprising a stent and a coating on the stent wherein the coating comprises PLGA and crystalline sirolimus and wherein following implantation into a subject endothelial function is maintained with no incidence of vasoconstriction at 9 months following implantation.

Provided herein is a device comprising a stent; and a coating on the stent; wherein the coating comprises at least one polymer and a macrolide immunosuppressive (limus) drug, wherein at least a portion of the macrolide immunosuppressive (limus) drug is in crystalline form; wherein an evaluation of the device following implantation determines that the majority of the proliferative response depicted by the magnitude of neointimal proliferation and strut coverage occurs in the first 28 days after implantation.

In some embodiments, an evaluation of the device following implantation determines that after the first 28 days following implantation, no statistically significant changes occur in the proportion of strut coverage and amount of neointimal hyperplasia at 90 and 180 days. In some embodiments, an evaluation of the device following implantation determines that substantially all post-procedure malapposition resolves by 28-day follow-up. In some embodiments, the evaluation is performed by OCT analysis. In some embodiments, an evaluation of the device following implantation showing a satisfactory healing response to the implantation of the device by histologically demonstrating low inflammation scores and complete endothelial coverage at 180 days in combination with the neointimal maturation at 28 days following implantation by OCT analysis.

Provided herein is a method comprising providing a coated stent comprising a stent; and a coating on the stent; wherein the coating comprises at least one polymer and at least one macrolide immunosuppressive (limus) drug, wherein at least a portion of the macrolide immunosuppressive (limus) drug is in crystalline form; and determining that the majority of the proliferative response depicted by the magnitude of neointimal proliferation and strut coverage occurs in the first 28 days after implantation.

In some embodiments, the method comprises determining that, after the first 28 days following implantation, no statistically significant changes occur in the proportion of strut coverage and amount of neointimal hyperplasia at 90 and 180 days. In some embodiments, the method comprises determining that substantially all post-procedure malapposition resolves by 28-day follow-up. In some embodiments, the method comprises determining that there is neointimal maturation 28 days following implantation. In some embodiments, the determining step is performed by OCT analysis. In some embodiments, the method comprises showing a satisfactory healing response to the implantation of the device by histologically demonstrating low inflammation scores and complete endothelial coverage at 180 days in combination with the neointimal maturation at 28 days following implantation by OCT analysis.

Provided herein is a device comprising a stent; and a coating on the stent; wherein the coating comprises at least one polymer and a macrolide immunosuppressive (limus) drug, wherein at least a portion of the macrolide immunosuppressive (limus) drug is in crystalline form; wherein the coating is cleared from the stent in about 45 to 60 days following implantation of the device in vivo, leaving a bare metal stent.

Provided herein is a device comprising a stent; and a coating on the stent; wherein the coating comprises at least one polymer and a macrolide immunosuppressive (limus) drug, wherein at least a portion of the macrolide immunosuppressive (limus) drug is in crystalline form; and wherein the polymer is fully absorbed by the tissue in at most 90 days following implantation of the device in vivo, leaving a bare metal stent.

In certain embodiments, clearance of the coating from the stent is shown by measuring the amount of drug on the stent. In certain embodiments, clearance of the coating from the stent occurs when at least one of: over 52% of the drug is no longer associated with the stent, at least 75% of the drug is no longer associated with the stent, at least 80% of the drug is no longer associated with the stent, at least 90% of the drug is no longer associated with the stent, at least 95% of the drug is no longer associated with the stent, and at least 97% of the drug is no longer associated with the stent.

In certain embodiments, the drug loading is from about 9 μg per unit stent length to about 12 μg per unit stent length. In certain embodiments, the drug loading is from 9 μg per unit stent length to 12 μg per unit stent length. In certain embodiments, the drug loading target ranges from about 75 μg to about 300 μg. In certain embodiments, drug loading target ranges from about 83 μg to about 280 μg. In certain embodiments, the drug loading target ranges from 75 μg to 300 μg. In certain embodiments, drug loading target ranges from 83 μg to 280 μg. In certain embodiments, the polymer is fully absorbed by the vessel by at most 90 days.

In certain embodiments, full absorption is when there is at least 75% absorption of the polymer by the tissue surrounding the stent, at least 80% absorption of the polymer by the tissue surrounding the stent, at least 90% absorption of the polymer by the tissue surrounding the stent, at least 95% absorption of the polymer by the tissue surrounding the stent, or 100% absorption of the polymer by the tissue surrounding the stent. In certain embodiments, full absorption is when there is no evidence of polymer in the tissue surrounding the stent after 90 days following implantation.

In certain embodiments, the coated stent is lubricious. In certain embodiments, the coated stent is hydriphilic. In certain embodiments, the stent is thin. In certain embodiments, struts of the stent are about 64 microns on average. In certain embodiments, imaging with OCT demonstrates thin, homogenous coverage of the stent with tissue 4 months after implantation with the device. In certain embodiments, imaging with OCT demonstrates >90% strut coverage with tissue 4 months after implantation with the device. In certain embodiments, imaging with OCT demonstrates >80% strut coverage with tissue 4 months after implantation with the device. In certain embodiments, imaging with OCT demonstrates no stent strut malapposition 4 months after implantation with the device. In certain embodiments, imaging with OCT demonstrates no stent strut malapposition 6 months after implantation with the device. In certain embodiments, imaging with OCT demonstrates no stent strut malapposition 8 months after implantation with the device. In certain embodiments, imaging with OCT demonstrates a low rate of stent strut malapposition 4 months after implantation with the device in a population of subjects comprising at least 5 subjects. In certain embodiments, imaging with OCT demonstrates a low rate of stent strut malapposition 6 months after implantation with the device in a population of subjects comprising at least 5 subjects. In certain embodiments, imaging with OCT demonstrates a low rate of stent strut malapposition 8 months after implantation with the device in a population of subjects comprising at least 5 subjects. In certain embodiments, imaging with OCT demonstrates a low rate of stent strut malapposition 18 months after implantation with the device in a population of subjects comprising at least 5 subjects.

In certain embodiments, there is minimal neointimal hyperplasia 4 months after implantation with the device. In certain embodiments, there is neointimal obstruction of no more than about 5.2% on average. In certain embodiments, there is minimal neointimal hyperplasia 6 months after implantation with the device. In certain embodiments, there is minimal neointimal hyperplasia 8 months after implantation with the device.

In certain embodiments, occurrence of late stent thrombosis is reduced as compared to other drug eluting stents. In certain embodiments, there is no indication of binary restenosis at 4 months after implantation with the device. In certain embodiments, there is no indication of binary restenosis at 6 months after implantation with the device. In certain embodiments, there is no indication of binary restenosis at 8 months after implantation with the device. In certain embodiments, there is no indication of binary restenosis at 18 months after implantation with the device.

In certain embodiments, there is minimal change in late stent loss between 4 and 8 months following implantation with the device. This shows sustained and effectively suppressed neointimal hyperplasia.

In certain embodiments, there is low neointimal hyperplasia by analysis of at least one of neointimal obstruction (%), neointimal volume index (mm³/mm), and late area loss (mm²) measured at 8 months following implantation with the device, as determined by IVUS.

In certain embodiments, the stent was coated using an RESS method. In certain embodiments, the RESS method uses a PDPDP sequence of steps to produce the coated stent. In certain embodiments, the PDPDP sequence of steps comprises Polymer single spray, sinter, Drug spray, Polymer double spray, sinter, Drug spray, Polymer triple spray, sinter. In certain embodiments, the PDPDP sequence of steps comprises a first Polymer spray, sinter, Drug spray, a second Polymer spray that is about twice as long as the first Polymer spray, sinter, Drug spray, third Polymer spray that is about three times as long as the first Polymer spray, sinter. In certain embodiments, the PDPDP sequence of steps comprises a first Polymer spray, sinter, Drug spray, a second Polymer spray that deposits about twice as much Polymer as the first Polymer spray, sinter, Drug spray, third Polymer spray deposits about three times as much Polymer as the first Polymer spray, sinter.

In certain embodiments, the Polymer comprises PLGA 50:50 having a number average molecular weight of about 15 kD.

In certain embodiments, implantation of the device results in rapid, uniform neointimal coverage with no adverse vessel reaction at four months follow up, at least. In certain embodiments, implantation of the device results late lumen loss and percent (%) obstruction which show good inhibition of neointimal hyperplasia. In certain embodiments, implantation of the device results in in-stent late lumen loss at 8 months of about 0.09 mm, the percent neointimal obstruction at 8 months of about 10.9%, and there are no incidences of binary restenosis or revascularizations. In certain embodiments, implantation of the device results in in-stent late lumen loss at 18 months of about 0.09 mm and the percent neointimal obstruction at 18 months of about 11.2%. In certain embodiments, implantation of the device results in in-stent late lumen loss at 18 months of about 0.09 mm, the percent neointimal obstruction at 18 months of about 11.2%, and there are no incidences of binary restenosis or revascularizations. In certain embodiments, after 4 months of implantation of the device, no significant changes are observed in vessel volume index, plaque volume index, or lumen volume index as compared to just after implantation. In certain embodiments, neointimal obstruction at 4 months is minimal and there is no significant lumen encroachment.

In certain embodiments, a majority of the stented segment is covered with IVUS-detectable neointima as early as 4 months following implantation with the device. In certain embodiments, at least 50% of the stented segment is covered with IVUS-detectable neointima as early as 4 months following implantation with the device. In certain embodiments, at least 60% of the stented segment is covered with IVUS-detectable neointima as early as 4 months following implantation with the device. In certain embodiments, at least 70% of the stented segment is covered with IVUS-detectable neointima as early as 4 months following implantation with the device. In certain embodiments, at least 80% of the stented segment is covered with IVUS-detectable neointima as early as 4 months following implantation with the device.

In certain embodiments, OCT demonstrates good strut coverage at 4 months, 6 months and 8 months following implantation of the device. In certain embodiments, OCT demonstrates strut coverage of at least 80% of the struts on average at each of 4 months, 6 months and 8 months following implantation of the device.

In certain embodiments, OCT demonstrates good strut coverage at 4 months, 6 months, 8 months, and 18 months following implantation of the device. In certain embodiments, OCT demonstrates strut coverage of at least 80% of the struts on average at each of 4 months, 6 months, 8 months, and 18 months following implantation of the device.

In certain embodiments, the device comprises an improved safety profile as compared to drug eluting stents made by other methods. In certain embodiments, the methods comprise solvent based coating methods. In certain embodiments, substantially all of the drug is amorphous in form on the stent of the other drug eluting stents.

In certain embodiments, the device comprises a controlled, continuous, sustained release of drug over 6 months in-vivo, without an initial drug burst into the tissue surrounding the device or into the blood stream.

In certain embodiments, the device mitigates hypersensitivity, impaired healing, and abnormal vasomotor function as compared to coated stents having longer absorption times or durable polymers thereon.

In certain embodiments, the device reduces risks of DAPT non-compliance and/or interruption as compared to other drug eluting stents.

In certain embodiments, the device reduces or eliminates risks of permanent coating such as long term thrombosis risks.

In certain embodiments, complete strut coverage is shown as early as 1 month following implantation. In certain embodiments, low intimal hyperplasia is shown up to 180 days following implantation, at least. In certain embodiments, no evidence of late catch up is shown at 180 days following implantation, at least. In certain embodiments, no stent malapposition was detected through 90 days. In certain embodiments, there is no late acquired malapposition detected in the implanted device.

In certain embodiments, drug is at least one of: 50% crystalline, at least 75% crystalline, at least 90% crystalline. In certain embodiments, the drug comprises at least one polymorph of the possible polymorphs of the crystalline structures of the drug.

In certain embodiments, the polymer comprises a bioabsorbable polymer. In certain embodiments, the polymer comprises PLGA. In certain embodiments, the polymer comprises PLGA with a ratio of about 40:60 to about 60:40. In certain embodiments, the polymer comprises PLGA with a ratio of about 40:60 to about 60:40 and further comprises PLGA with a ratio of about 60:40 to about 90:10. In certain embodiments, the polymer comprises PLGA having a weight average molecular weight of about 10 kD and wherein the coating further comprises PLGA having a weight average molecular weight of about 19 kD. In certain embodiments, the polymer is selected from the group: PLGA, a copolymer comprising PLGA (i.e. a PLGA copolymer), a PLGA copolymer with a ratio of about 40:60 to about 60:40, a PLGA copolymer with a ratio of about 70:30 to about 90:10, a PLGA copolymer having a weight average molecular weight of about 10 kD, a PLGA copolymer having a weight average molecular weight of about 19 kD, PGA poly(glycolide), LPLA poly(l-lactide), DLPLA poly(dl-lactide), PCL poly(e-caprolactone) PDO, poly(dioxolane) PGA-TMC, 85/15 DLPLG p(dl-lactide-co-glycolide), 75/25 DLPLG, 65/35 DLPLG, 50/50 DLPLG, TMC poly(trimethylcarbonate), poly(anhydrides) such as p(CPP:SA) poly(1,3-bis-p-(carboxyphenoxy)propane-co-sebacic acid), and a combination thereof.

In certain embodiments, the stent comprises a cobalt-chromium alloy. In certain embodiments, the stent is formed from a material comprising the following percentages by weight: about 0.05 to about 0.15 C, about 1.00 to about 2.00 Mn, about 0.04 Si, about 0.03 P, about 0.3 S, about 19.0 to about 21.0 Cr, about 9.0 to about 11.0 Ni, about 14.0 to about 16.00 W, about 3.0 Fe, and Bal. Co. In certain embodiments, the stent is formed from a material comprising at most the following percentages by weight: about 0.025 C, about 0.15 Mn, about 0.15 Si, about 0.015 P, about 0.01 S, about 19.0 to about 21.0 Cr, about 33 to about 37 Ni, about 9.0 to about 10.5 Mo, about 1.0 Fe, about 1.0 Ti, and Bal. Co. In certain embodiments, the stent is formed from a material comprising a platinum chromium alloy.

In certain embodiments, the stent has a thickness of from about 50% to about 90% of a total thickness of the device. In certain embodiments, the coating has a total thickness of from about 5 μm to about 50 μm.

In some embodiments, the device has an active agent content of from about 5 μg to about 500 μg. In certain embodiments, the device has an active agent content of from about 100 μg to about 160 μg.

In certain embodiments, the macrolide immunosuppressive drug comprises one or more of: rapamycin, biolimus (biolimus A9), 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′:E,4′S)-40-O-(4′,5′-Dihydroxypent-2′-en-1′-yl)-rapamycin, 40-O-(2-Hydroxy)ethoxycar-bonylmethyl-rapamycin, 40-O-(3-Hydroxyl)propyl-rapamycin, 40-O-(6-Hydroxyl)hexyl-rapamycin, 40-O-[2-(2-Hydroxyl)ethoxy]ethyl-rapamycin, 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin 40-O-(2-Nicotinoyloxy)ethyl-rapamycin, 40-O-[2-(N-Morpholino)acetoxy]ethyl-rapamycin, 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxyl)ethyl-rapamycin, 28-O-Methyl-rapamycin, 40-O-(2-Aminoethyl)-rapamycin, 40-O-(2-Acetaminoethyl)-rapamycin, 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl)rapamycin (tacrolimus), 42-[3-hydroxy-2-(hydroxymethyl)-2-methylpropanoate]rapamycin (temsirolimus), (42S)-42-Deoxy-42-(1H-tetrazol-1-yl)-rapamycin (zotarolimus), and salts, derivatives, isomers, racemates, diastereoisomers, prodrugs, hydrate, ester, or analogs thereof.

Provided herein is a method comprising providing a coated stent comprising a stent; and a coating on the stent; wherein the coating comprises at least one polymer and at least one macrolide immunosuppressive (limus) drug, wherein at least a portion of the macrolide immunosuppressive (limus) drug is in crystalline form; and wherein the coating is cleared from the stent in about 45 to 60 days following implantation of the device in vivo, leaving a bare metal stent.

Provided herein is a method comprising providing a coated stent comprising a stent; and a coating on the stent; wherein the coating comprises at least one polymer and at least one macrolide immunosuppressive (limus) drug, wherein at least a portion of the macrolide immunosuppressive (limus) drug is in crystalline form; and wherein the polymer is fully absorbed by the tissue in at most 90 days following implantation of the device in vivo, leaving a bare metal stent.

In some embodiments, the drug is present in the vessel at about 90 days following implantation, at about 180 days following implantation, and/or at about 365 days following implantation. In some embodiments, the drug is present in the vessel at 90 days following implantation. In some embodiments, the drug is present in the vessel at 180 days following implantation. In some embodiments, the drug is present in the vessel at 365 days following implantation.

Provided herein is a method of coating a stent comprising: mounting a stent on a holder in a coating chamber that imparts a charge to the stent, providing a first cloud of charged particles of polymer to the stents by rapidly expanding a pressurized solution of the polymer in densified 1,1,1,2,3,3-hexafluoropropane through a first orifice, wherein the polymer comprises PLGA, wherein a first polymer layer of the polymer particles is formed on the stent by electrostatic deposition, sintering the first polymer layer at >40° C. in ambient pressure, providing a first cloud of charged sirolimus particles to the stents having an opposite charge than the charge of the stent by pulsing sirolimus particles into the chamber using a propellant in order to deposit a first agent layer on the stent, wherein at least a portion of the sirolimus particles is in crystalline form, providing a second cloud of charged particles of the polymer and a third cloud of charged particles of the polymer to the stents by sequentially rapidly expanding the pressurized solution through the first orifice, wherein the particles have an opposite charge than the charge of the stent, wherein a second polymer layer of the polymer particles is formed on the stent by electrostatic deposition, sintering the second polymer layer at >40° C. in ambient pressure, providing a second cloud of charged sirolimus particles to the stents having an opposite charge than the charge of the stent by pulsing the sirolimus particles into the chamber using a propellant in order to deposit a second agent layer on the stent, wherein at least a portion of the sirolimus particles is in crystalline form, providing a fourth cloud of charged particles of the polymer, a fifth cloud of charged particles of the polymer, and a sixth cloud of charged particles of the polymer to the stents by sequentially rapidly expanding a pressurized solution through the first orifice, wherein the particles have an opposite charge than the charge of the stent, wherein a third polymer layer of the polymer particles is formed on the stent by electrostatic deposition, and sintering the third polymer layer at >40° C. 150 psi pressurization with gaseous 1,1,1,2,3,3-hexafluoropropane, wherein the crystalline form sirolimus particles in the first agent layer and second agent layer remain in crystalline form throughout all steps in the method. In some embodiments the particles have an opposite charge than the charge of the stent. In some embodiments the sintering is performed at about 100° C., or at 100° C.

In some embodiments, the stent on the holder is orbiting through any of the first, second third, fourth, fifth, or sixth clouds of charged polymer particles, or through any of the first or second clouds of charged sirolimus particles.

In some embodiments, the first orifice is heated sufficiently to overcome Jould-Thompson cooling. In some embodiments, the first orifice is heated sufficiently to ensure that the compressed gas is fully vaporized on expansion from the orifice.

In some embodiments, the concentration of the solution is any of 2 w/v % (weight or mass of polymer per total volume), 4 w/v %, about 2 w/v %, about 4 w/v %, about 2 w/v % to about 4 w/v %, 2 w/v % to 4 w/v %, 2 w/v %+/−0.5 w/v %, 2 w/v %+/−0.25 w/v %, 2 w/v %+/−0.1 w/v %, 4 w/v %+/−0.5 w/v %, 4 w/v %+/−0.25 w/v %, 4 w/v %+/−0.1 w/v %, at least 1 w/v %, at least 1.5 w/v %, at least 2 w/v %, at least 3 w/v %, at least 4 w/v %, at most 4 w/v %, at most 5 w/v %, at most 6 w/v %, at most 7 w/v %, at most 8 w/v %, at most 9 w/v %, at most 10 w/v %, at most 11 w/v %, at most 12 w/v %, at most 13 w/v %, at most 14 w/v %, or at most 15 w/v %.

In some embodiments, the flow rate is controlled and fixed using an automated syringe pump.

In some embodiments, the charge of the polymer or active agent particles is oppositely polarized as compared to the stent and comprises a potential of any of ±1.0 kV, ±1.2 kV, ±1.3 kV, ±1.4 kV, ±1.5 kV, ±1.6 kV, ±1.7 kV, ±1.8 kV, ±1.9 kV, ±2 kV, ±3 kV, ±3.5 kV, ±4 kV, ±5 kV, from ±1.0 kV to ±2.0 kV, from ±1.2 kV to ±1.8 kV, from ±1.4 kV to ±1.6 kV, from ±0.5 kV to ±5 kV, or about ±1.5 kV.

In some embodiments, the sirolimus particles have been micronized prior to introduction into the chamber. In some embodiments, the sirolimus particles comprise a particle distribution such that at least 99% by volume of the sirolimus particles are less than 10 microns with the distribution centered at 2.75+/−0.5 microns. In some embodiments, the sirolimus particles comprise a particle distribution such that 80%, 85%, 90%, 95%, 99%, at least 50%, at least 75%, at least 80%, at least 85%, at least 90%, at least 95%, at least 99%, at least about 50%, at least about 75%, at least about 80%, at least about 85%, at least about 90%, at least about 95%, or at least about 99% by volume of the particles are less than 10 microns. In some embodiments, the sirolimus particles comprise a particle distribution such that at least 50% by volume of the particles are less than 3 microns, less than 5 microns, less than 7.5 microns, less than 10 microns, less than 20 microns, less than 25 microns, less than 30 microns, less than 40 microns, less than 50 microns, less than 75 microns, less than about 10 microns, less than about 15 microns, or less than about 7.5 microns. In some embodiments, the sirolimus particles have a distribution centered at 1.0+/−0.5 microns, 1.25+/−0.5 microns, 1.5+/−0.5 microns, 1.75+/−0.5 microns, 2.0+/−0.5 microns, 2.25+/−0.5 microns, 2.5+/−0.5 microns, 2.75+/−0.5 microns, 3.0+/−0.5 microns, 3.25+/−0.5 microns, 3.5+/−0.5 microns, 3.75+/−0.5 microns, 4.0+/−0.5 microns, 4.25+/−0.5 microns, 4.5+/−0.5 microns, 4.75+/−0.5 microns, 5+/−0.5 microns, 5.5+/−0.5 microns, 6+/−0.5 microns, 6.5+/−0.5 microns, 7+/−0.5 microns, 7.5+/−0.5 microns, 8+/−0.5 microns, 8.5+/−0.5 microns, 9+/−0.5 microns, 10+/−0.5 microns, 15+/−0.5 microns, 20+/−0.5 microns, 25+/−0.5 microns, 30+/−0.5 microns, 35+/−0.5 microns, 40+/−0.5 microns, 45+/−0.5 microns, 50+/−0.5 microns, about 1.0 microns, about 1.5 microns, about 2.0 microns, about 2.5 microns, about 2.75 microns, about 3.0 microns, about 3.5 microns, about 4.0 microns, about 4.5 microns, about 5 microns, about 6 microns, about 7 microns, about 8 microns, about 9 microns, about 10 microns, about 15 microns, about 20 microns, about 25 microns, about 30 microns, about 35 microns, about 40 microns, about 45 microns, or about 50 microns.

In some embodiments, the propellant comprises a noble gas. In some embodiments, the noble gas comprises argon, nitrogen or helium. In some embodiments, the propellant is pressurized to at least 50 psi, at least 75 psi, at least 100 psi, at least 150 psi, at least 200 psi, at least 250 psi, at least 300 psi, about 50 psi, about 75 psi, about 100 psi, about 150 psi, about 200 psi, about 250 psi, about 300 psi, about 350 psi, about 400 psi, about 450 psi, about 500 psi, about 550 psi, about 600 psi, 50 psi to 500 psi, 200 psi to 400 psi, 250 psi to 350 psi, 50 psi, 75 psi, 100 psi, 150 psi, 200 psi, 250 psi, 300 psi, 350 psi, 400 psi, 450 psi, 500 psi, 550 psi, or 600 psi.

Provided herein is a device comprising a stent and a coating on the stent wherein the coating comprises PLGA and crystalline sirolimus and wherein the stent is made by any one of the methods described herein.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts an embodiment of micronized sirolimus used in a spray coating process described in Example 1, at least

FIG. 2 shows release profile of sirolimus from the Sirolimus DES.

FIG. 3 shows the Patient level in-stent LLL by follow-up group, indicating no binary restenosis and having a linear regression indicating minimal change in LLL between 4 and 8 months as tested in the study of Example 2.

FIG. 4 shows a target artery and lesion of a single patient from the study of Example 2 viewed by IVUS at 8 months follow up.

FIG. 5 shows a histogram of Neointimal obstruction of devices of Example 2 at 4 months follow up as tested and analyzed using IVUS.

FIG. 6 shows Vessel Response in Example 2, which shows Vessel Volume Index, Plaque Volume Index, and Lumen Volume Index at baseline (at implantation) and at 4 months follow up.

FIG. 7 shows the target artery and lesion of a single patient viewed under fluoroscopy prior to implantation of the device from the study of Example 2, just after implantation, and at 8 months follow up.

FIG. 8 depicts the results of neointimal thickness in mm, including statistical results comparing the groups as noted, as described in Example 3, at least.

FIG. 9 depicts the results of uncovered struts in %, including statistical results comparing the groups as noted, as described in Example 3, at least.

FIG. 10 depicts the results of malapposed struts in %, including statistical results comparing the groups as noted, as described in Example 3, at least.

FIG. 11 shows the neointimal thickness score and standard deviation recorded at each of 30 days and 90 days in both a single and overlapping (OLP) Sirolimus DES (left column at each time point and condition) and Vision BMS (right column at each time point and condition) stent implantation in a porcine model.

FIG. 12 shows the average inflammation score and standard deviation recorded at each of 30 days and 90 days in both a single and overlapping (OLP) Sirolimus DES (left column at each time point and condition) and Vision BMS (right column at each time point and condition) stent implantation in a porcine model.

FIG. 13 depicts in-stent late lumen loss as described in Example 4.

FIG. 14 depicts neointimal obstruction as described in Example 4.

FIG. 15 depicts maximum cross-sectional narrowing as described in Example 4.

FIG. 16 shows a comparison of Test device (labeled as Sirolimus DES) and Endeavor® Sprint ZES in late lumen loss as described in Example 5.

### DETAILED DESCRIPTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments contemplated herein will be apparent to those skilled in the art in light of the instant disclosure, which do not depart from the instant invention. Hence, the following specification is intended to illustrate selected embodiments of the invention, and not to exhaustively specify all permutations, combinations and variations thereof.

### DEFINITIONS

As used in the present specification, the following words and phrases are generally intended to have the meanings as set forth below, except to the extent that the context in which they are used indicates otherwise.

“Substrate” as used herein, refers to any surface upon which it is desirable to deposit a coating comprising a polymer and a pharmaceutical or biological agent, wherein the coating process does not substantially modify the morphology of the pharmaceutical agent or the activity of the biological agent. Biomedical implants are of particular interest for the present invention; however the present invention is not intended to be restricted to this class of substrates. Those of skill in the art will appreciate alternate substrates that could benefit from the coating process described herein, such as pharmaceutical tablet cores, as part of an assay apparatus or as components in a diagnostic kit (e.g. a test strip).

“Biomedical implant” as used herein refers to any implant for insertion into the body of a human or animal subject, including but not limited to stents (e.g., coronary stents, vascular stents including peripheral stents and graft stents, urinary tract stents, urethral/prostatic stents, rectal stent, oesophageal stent, biliary stent, pancreatic stent), electrodes, catheters, leads, implantable pacemaker, cardioverter or defibrillator housings, joints, screws, rods, ophthalmic implants, femoral pins, bone plates, grafts, anastomotic devices, perivascular wraps, sutures, staples, shunts for hydrocephalus, dialysis grafts, colostomy bag attachment devices, ear drainage tubes, leads for pace makers and implantable cardioverters and defibrillators, vertebral disks, bone pins, suture anchors, hemostatic barriers, clamps, screws, plates, clips, vascular implants, tissue adhesives and sealants, tissue scaffolds, various types of dressings (e.g., wound dressings), bone substitutes, intraluminal devices, vascular supports, etc.

The implants may be formed from any suitable material, including but not limited to polymers (including stable or inert polymers, organic polymers, organic-inorganic copolymers, inorganic polymers, and biodegradable polymers), metals, metal alloys, inorganic materials such as silicon, and composites thereof, including layered structures with a core of one material and one or more coatings of a different material. Substrates made of a conducting material facilitate electrostatic capture. However, the invention contemplates the use of electrostatic capture, as described below, in conjunction with substrate having low conductivity or which are non-conductive. To enhance electrostatic capture when a non-conductive substrate is employed, the substrate is processed for example while maintaining a strong electrical field in the vicinity of the substrate.

Subjects into which biomedical implants of the invention may be applied or inserted include both human subjects (including male and female subjects and infant, juvenile, adolescent, adult and geriatric subjects) as well as animal subjects (including but not limited to pig, rabbit, mouse, dog, cat, horse, monkey, etc.) for veterinary purposes and/or medical research.

In a preferred embodiment the biomedical implant is an expandable intraluminal vascular graft or stent that can be expanded within a blood vessel by an angioplasty balloon associated with a catheter to dilate and expand the lumen of a blood vessel, such as described in U.S. Pat. No. 4,733,665 to Palmaz.

“Pharmaceutical agent” as used herein refers to any of a variety of drugs or pharmaceutical compounds that can be used as active agents to prevent or treat a disease (meaning any treatment of a disease in a mammal, including preventing the disease, i.e. causing the clinical symptoms of the disease not to develop; inhibiting the disease, i.e. arresting the development of clinical symptoms; and/or relieving the disease, i.e. causing the regression of clinical symptoms). It is possible that the pharmaceutical agents of the invention may also comprise two or more drugs or pharmaceutical compounds. Pharmaceutical agents, include but are not limited to antirestenotic agents, antidiabetics, analgesics, antiinflammatory agents, antirheumatics, antihypotensive agents, antihypertensive agents, psychoactive drugs, tranquillizers, antiemetics, muscle relaxants, glucocorticoids, agents for treating ulcerative colitis or Crohn's disease, antiallergics, antibiotics, antiepileptics, anticoagulants, antimycotics, antitussives, arteriosclerosis remedies, diuretics, proteins, peptides, enzymes, enzyme inhibitors, gout remedies, hormones and inhibitors thereof, cardiac glycosides, immunotherapeutic agents and cytokines, laxatives, lipid-lowering agents, migraine remedies, mineral products, otologicals, anti parkinson agents, thyroid therapeutic agents, spasmolytics, platelet aggregation inhibitors, vitamins, cytostatics and metastasis inhibitors, phytopharmaceuticals, chemotherapeutic agents and amino acids. Examples of suitable active ingredients are acarbose, antigens, beta-receptor blockers, non-steroidal antiinflammatory drugs [NSAIDs], cardiac glycosides, acetylsalicylic acid, virustatics, aclarubicin, acyclovir, cisplatin, actinomycin, alpha- and beta-sympatomimetics, (dimeprazole, allopurinol, alprostadil, prostaglandins, amantadine, ambroxol, amlodipine, methotrexate, S-amino salicylic acid, amitriptyline, amoxicillin, anastrozole, atenolol, azathioprine, balsalazide, beclomethasone, betahistine, bezafibrate, bicalutamide, diazepam and diazepam derivatives, budesonide, bufexamac, buprenorphine, methadone, calcium salts, potassium salts, magnesium salts, candesartan, carbamazepine, captopril, cefalosporins, cetirizine, chenodeoxycholic acid, ursodeoxycholic acid, theophylline and theophylline derivatives, trypsins, cimetidine, clarithromycin, clavulanic acid, clindamycin, clobutinol, clonidine, cotrimoxazole, codeine, caffeine, vitamin D and derivatives of vitamin D, colestyramine, cromoglicic acid, coumarin and coumarin derivatives, cysteine, cytarabine, cyclophosphamide, ciclosporin, cyproterone, cytabarine, dapiprazole, desogestrel, desonide, dihydralazine, diltiazem, ergot alkaloids, dimenhydrinate, dimethyl sulphoxide, dimethicone, domperidone and domperidan derivatives, dopamine, doxazosin, doxorubicin, doxylamine, dapiprazole, benzodiazepines, diclofenac, glycoside antibiotics, desipramine, econazole, ACE inhibitors, enalapril, ephedrine, epinephrine, epoetin and epoetin derivatives, morphinans, calcium antagonists, irinotecan, modafinil, orlistat, peptide antibiotics, phenytoin, riluzoles, risedronate, sildenafil, topiramate, macrolide antibiotics, oestrogen and oestrogen derivatives, progestogen and progestogen derivatives, testosterone and testosterone derivatives, androgen and androgen derivatives, ethenzamide, etofenamate, etofibrate, fenofibrate, etofylline, etoposide, famciclovir, famotidine, felodipine, fenofibrate, fentanyl, fenticonazole, gyrase inhibitors, fluconazole, fludarabine, fluarizine, fluorouracil, fluoxetine, flurbiprofen, ibuprofen, flutamide, fluvastatin, follitropin, formoterol, fosfomicin, furosemide, fusidic acid, gallopamil, ganciclovir, gemfibrozil, gentamicin, ginkgo, Saint John's wort, glibenclamide, urea derivatives as oral antidiabetics, glucagon, glucosamine and glucosamine derivatives, glutathione, glycerol and glycerol derivatives, hypothalamus hormones, goserelin, gyrase inhibitors, guanethidine, halofantrine, haloperidol, heparin and heparin derivatives, hyaluronic acid, hydralazine, hydrochlorothiazide and hydrochlorothiazide derivatives, salicylates, hydroxyzine, idarubicin, ifosfamide, imipramine, indometacin, indoramine, insulin, interferons, iodine and iodine derivatives, isoconazole, isoprenaline, glucitol and glucitol derivatives, itraconazole, ketoconazole, ketoprofen, ketotifen, lacidipine, lansoprazole, levodopa, levomethadone, thyroid hormones, lipoic acid and lipoic acid derivatives, lisinopril, lisuride, lofepramine, lomustine, loperamide, loratadine, maprotiline, mebendazole, mebeverine, meclozine, mefenamic acid, mefloquine, meloxicam, mepindolol, meprobamate, meropenem, mesalazine, mesuximide, metamizole, metformin, methotrexate, methylphenidate, methylprednisolone, metixene, metoclopramide, metoprolol, metronidazole, mianserin, miconazole, minocycline, minoxidil, misoprostol, mitomycin, mizolastine, moexipril, morphine and morphine derivatives, evening primrose, nalbuphine, naloxone, tilidine, naproxen, narcotine, natamycin, neostigmine, nicergoline, nicethamide, nifedipine, niflumic acid, nimodipine, nimorazole, nimustine, nisoldipine, adrenaline and adrenaline derivatives, norfloxacin, novamine sulfone, noscapine, nystatin, ofloxacin, olanzapine, olsalazine, omeprazole, omoconazole, ondansetron, oxaceprol, oxacillin, oxiconazole, oxymetazoline, pantoprazole, paracetamol, paroxetine, penciclovir, oral penicillins, pentazocine, pentifylline, pentoxifylline, perphenazine, pethidine, plant extracts, phenazone, pheniramine, barbituric acid derivatives, phenylbutazone, phenytoin, pimozide, pindolol, piperazine, piracetam, pirenzepine, piribedil, piroxicam, pramipexole, pravastatin, prazosin, procaine, promazine, propiverine, propranolol, propyphenazone, prostaglandins, protionamide, proxyphylline, quetiapine, quinapril, quinaprilat, ramipril, ranitidine, reproterol, reserpine, ribavirin, rifampicin, risperidone, ritonavir, ropinirole, roxatidine, roxithromycin, ruscogenin, rutoside and rutoside derivatives, sabadilla, salbutamol, salmeterol, scopolamine, selegiline, sertaconazole, sertindole, sertralion, silicates, sildenafil, simvastatin, sitosterol, sotalol, spaglumic acid, sparfloxacin, spectinomycin, spiramycin, spirapril, spironolactone, stavudine, streptomycin, sucralfate, sufentanil, sulbactam, sulphonamides, sulfasalazine, sulpiride, sultamicillin, sultiam, sumatriptan, suxamethonium chloride, tacrine, tacrolimus, taliolol, tamoxifen, taurolidine, tazarotene, temazepam, teniposide, tenoxicam, terazosin, terbinafine, terbutaline, terfenadine, terlipressin, tertatolol, tetracyclins, teryzoline, theobromine, theophylline, butizine, thiamazole, phenothiazines, thiotepa, tiagabine, tiapride, propionic acid derivatives, ticlopidine, timolol, tinidazole, tioconazole, tioguanine, tioxolone, tiropramide, tizanidine, tolazoline, tolbutamide, tolcapone, tolnaftate, tolperisone, topotecan, torasemide, antioestrogens, tramadol, tramazoline, trandolapril, tranylcypromine, trapidil, trazodone, triamcinolone and triamcinolone derivatives, triamterene, trifluperidol, trifluridine, trimethoprim, trimipramine, tripelennamine, triprolidine, trifosfamide, tromantadine, trometamol, tropalpin, troxerutine, tulobuterol, tyramine, tyrothricin, urapidil, ursodeoxycholic acid, chenodeoxycholic acid, valaciclovir, valproic acid, vancomycin, vecuronium chloride, Viagra, venlafaxine, verapamil, vidarabine, vigabatrin, viloazine, vinblastine, vincamine, vincristine, vindesine, vinorelbine, vinpocetine, viquidil, warfarin, xantinol nicotinate, xipamide, zafirlukast, zalcitabine, zidovudine, zolmitriptan, zolpidem, zoplicone, zotipine and the like. See, e.g., U.S. Pat. No. 6,897,205; see also U.S. Pat. No. 6,838,528; U.S. Pat. No. 6,497,729, incorporated herein by reference in their entirety.

Examples of therapeutic agents employed in conjunction with the invention include, rapamycin, biolimus (biolimus A9), 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′:E,4′S)-40-O-(4′,5′-Dihydroxypent-2′-en-1′-yl)-rapamycin, 40-O-(2-Hydroxy)ethoxycar-bonylmethyl-rapamycin, 40-O-(3-Hydroxyl)propyl-rapamycin, 40-O-(6-Hydroxyl)hexyl-rapamycin, 40-O-[2-(2-Hydroxyl)ethoxy]ethyl-rapamycin, 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin 40-O-(2-Nicotinoyloxy)ethyl-rapamycin, 40-O-[2-(N-Morpholino)acetoxy]ethyl-rapamycin, 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxyl)ethyl-rapamycin, 28-O-Methyl-rapamycin, 40-O-(2-Aminoethyl)-rapamycin, 40-O-(2-Acetaminoethyl)-rapamycin, 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl)rapamycin (tacrolimus), 42-[3-hydroxy-2-(hydroxymethyl)-2-methylpropanoate]rapamycin (temsirolimus).

As used herein, the pharmaceutical agent sirolimus may also and/or alternatively be called rapamycin, or vice versa, unless otherwise noted with regard to a particular term—for nonlimiting example, 42-Epi-(tetrazolyl)rapamycin is tacrolimus as noted herein.

The pharmaceutical agents may, if desired, also be used in the form of their pharmaceutically acceptable salts or derivatives (meaning salts which retain the biological effectiveness and properties of the compounds of this invention and which are not biologically or otherwise undesirable), and in the case of chiral active ingredients it is possible to employ both optically active isomers and racemates or mixtures of diastereoisomers. As well, the pharmaceutical agent may include a prodrug, a hydrate, an ester, a derivative or analogs of a compound or molecule.

In some embodiments, the pharmaceutical agent is, at least in part, crystalline. As used herein, the term crystalline may include any number of the possible polymorphs of the crystalline form of the pharmaceutical agent, including for non-limiting example a single polymorph of the pharmaceutical agent, or a plurality of polymorphs of the pharmaceutical agent. The crystalline pharmaceutical agent (which may include a semi-crystalline form of the pharmaceutical agent, depending on the embodiment) may comprise a single polymorph of the possible polymorphs of the pharmaceutical agent. The crystalline pharmaceutical agent (which may include a semi-crystalline form of the pharmaceutical agent, depending on the embodiment) may comprise a plurality of polymorphs of the possible polymorphs of the crystalline pharmaceutical agent. The polymorph, in some embodiments, is a packing polymorph, which exists as a result of difference in crystal packing as compared to another polymorph of the same crystalline pharmaceutical agent. The polymorph, in some embodiments, is a conformational polymorph, which is conformer of another polymorph of the same crystalline pharmaceutical agent. The polymorph, in some embodiments, is a pseudopolymorph. The polymorph, in some embodiments, is any type of polymorph—that is, the type of polymorph is not limited to only a packing polymorph, conformational polymorph, and/or a pseudopolymorph. When referring to a particular pharmaceutical agent herein which is at least in part crystalline, it is understood that any of the possible polymorphs of the pharmaceutical agent are contemplated.

A “pharmaceutically acceptable salt” may be prepared for any pharmaceutical agent having a functionality capable of forming a salt, for example an acid or base functionality. Pharmaceutically acceptable salts may be derived from organic or inorganic acids and bases. The term “pharmaceutically-acceptable salts” in these instances refers to the relatively non-toxic, inorganic and organic base addition salts of the pharmaceutical agents.

“Prodrugs” are derivative compounds derivatized by the addition of a group that endows greater solubility to the compound desired to be delivered. Once in the body, the prodrug is typically acted upon by an enzyme, e.g., an esterase, amidase, or phosphatase, to generate the active compound.

“Stability” as used herein in refers to the stability of the drug in a polymer coating deposited on a substrate in its final product form (e.g., stability of the drug in a coated stent). The term stability will define 5% or less degradation of the drug in the final product form.

“Active biological agent” as used herein refers to a substance, originally produced by living organisms, that can be used to prevent or treat a disease (meaning any treatment of a disease in a mammal, including preventing the disease, i.e. causing the clinical symptoms of the disease not to develop; inhibiting the disease, i.e. arresting the development of clinical symptoms; and/or relieving the disease, i.e. causing the regression of clinical symptoms). It is possible that the active biological agents of the invention may also comprise two or more active biological agents or an active biological agent combined with a pharmaceutical agent, a stabilizing agent or chemical or biological entity. Although the active biological agent may have been originally produced by living organisms, those of the present invention may also have been synthetically prepared, or by methods combining biological isolation and synthetic modification. By way of a non-limiting example, a nucleic acid could be isolated form from a biological source, or prepared by traditional techniques, known to those skilled in the art of nucleic acid synthesis. Furthermore, the nucleic acid may be further modified to contain non-naturally occurring moieties. Non-limiting examples of active biological agents include peptides, proteins, enzymes, glycoproteins, nucleic acids (including deoxyribonucleotide or ribonucleotide polymers in either single or double stranded form, and unless otherwise limited, encompasses known analogues of natural nucleotides that hybridize to nucleic acids in a manner similar to naturally occurring nucleotides), antisense nucleic acids, fatty acids, antimicrobials, vitamins, hormones, steroids, lipids, polysaccharides, carbohydrates and the like. They further include, but are not limited to, antirestenotic agents, antidiabetics, analgesics, antiinflammatory agents, antirheumatics, antihypotensive agents, antihypertensive agents, psychoactive drugs, tranquillizers, antiemetics, muscle relaxants, glucocorticoids, agents for treating ulcerative colitis or Crohn's disease, antiallergics, antibiotics, antiepileptics, anticoagulants, antimycotics, antitussives, arteriosclerosis remedies, diuretics, proteins, peptides, enzymes, enzyme inhibitors, gout remedies, hormones and inhibitors thereof, cardiac glycosides, immunotherapeutic agents and cytokines, laxatives, lipid-lowering agents, migraine remedies, mineral products, otologicals, anti parkinson agents, thyroid therapeutic agents, spasmolytics, platelet aggregation inhibitors, vitamins, cytostatics and metastasis inhibitors, phytopharmaceuticals and chemotherapeutic agents. Preferably, the active biological agent is a peptide, protein or enzyme, including derivatives and analogs of natural peptides, proteins and enzymes. The active biological agent may also be a hormone, gene therapies, RNA, siRNA, and/or cellular therapies (for non-limiting example, stem cells or T-cells).

“Active agent” as used herein refers to any pharmaceutical agent or active biological agent as described herein.

“Activity” as used herein refers to the ability of a pharmaceutical or active biological agent to prevent or treat a disease (meaning any treatment of a disease in a mammal, including preventing the disease, i.e. causing the clinical symptoms of the disease not to develop; inhibiting the disease, i.e. arresting the development of clinical symptoms; and/or relieving the disease, i.e. causing the regression of clinical symptoms). Thus the activity of a pharmaceutical or active biological agent should be of therapeutic or prophylactic value.

“Secondary, tertiary and quaternary structure” as used herein are defined as follows. The active biological agents of the present invention will typically possess some degree of secondary, tertiary and/or quaternary structure, upon which the activity of the agent depends. As an illustrative, non-limiting example, proteins possess secondary, tertiary and quaternary structure. Secondary structure refers to the spatial arrangement of amino acid residues that are near one another in the linear sequence. The α-helix and the β-strand are elements of secondary structure. Tertiary structure refers to the spatial arrangement of amino acid residues that are far apart in the linear sequence and to the pattern of disulfide bonds. Proteins containing more than one polypeptide chain exhibit an additional level of structural organization. Each polypeptide chain in such a protein is called a subunit. Quaternary structure refers to the spatial arrangement of subunits and the nature of their contacts. For example hemoglobin consists of two α and two β chains. It is well known that protein function arises from its conformation or three dimensional arrangement of atoms (a stretched out polypeptide chain is devoid of activity). Thus one aspect of the present invention is to manipulate active biological agents, while being careful to maintain their conformation, so as not to lose their therapeutic activity.

“Polymer” as used herein, refers to a series of repeating monomeric units that have been cross-linked or polymerized. Any suitable polymer can be used to carry out the present invention. It is possible that the polymers of the invention may also comprise two, three, four or more different polymers. In some embodiments, of the invention only one polymer is used. In some preferred embodiments a combination of two polymers are used. Combinations of polymers can be in varying ratios, to provide coatings with differing properties. Those of skill in the art of polymer chemistry will be familiar with the different properties of polymeric compounds.

Polymers useful in the devices and methods of the present invention include, for example, stable polymers, biostable polymers, durable polymers, inert polymers, organic polymers, organic-inorganic copolymers, inorganic polymers, bioabsorbable, bioresorbable, resorbable, degradable, and biodegradable polymers. These categories of polymers may, in some cases, be synonymous, and is some cases may also and/or alternatively overlap. Those of skill in the art of polymer chemistry will be familiar with the different properties of polymeric compounds.

In some embodiments, the coating comprises a polymer. In some embodiments, the active agent comprises a polymer. In some embodiments, the polymer comprises at least one of polyalkyl methacrylates, polyalkylene-co-vinyl acetates, polyalkylenes, polyurethanes, polyanhydrides, aliphatic polycarbonates, polyhydroxyalkanoates, silicone containing polymers, polyalkyl siloxanes, aliphatic polyesters, polyglycolides, polylactides, polylactide-co-glycolides, poly(e-caprolactone)s, polytetrahalooalkylenes, polystyrenes, poly(phosphasones), copolymers thereof, and combinations thereof.

Examples of polymers that may be used in the present invention include, but are not limited to polycarboxylic acids, cellulosic polymers, proteins, polypeptides, polyvinylpyrrolidone, maleic anhydride polymers, polyamides, polyvinyl alcohols, polyethylene oxides, glycosaminoglycans, polysaccharides, polyesters, aliphatic polyesters, polyurethanes, polystyrenes, copolymers, silicones, silicone containing polymers, polyalkyl siloxanes, polyorthoesters, polyanhydrides, copolymers of vinyl monomers, polycarbonates, polyethylenes, polypropytenes, polylactic acids, polylactides, polyglycolic acids, polyglycolides, polylactide-co-glycolides, polycaprolactones, poly(e-caprolactone)s, polyhydroxybutyrate valerates, polyacrylamides, polyethers, polyurethane dispersions, polyacrylates, acrylic latex dispersions, polyacrylic acid, polyalkyl methacrylates, polyalkylene-co-vinyl acetates, polyalkylenes, aliphatic polycarbonates polyhydroxyalkanoates, polytetrahalooalkylenes, poly(phosphasones), polytetrahalooalkylenes, poly(phosphasones), and mixtures, combinations, and copolymers thereof.

The polymers of the present invention may be natural or synthetic in origin, including gelatin, chitosan, dextrin, cyclodextrin, Poly(urethanes), Poly(siloxanes) or silicones, Poly(acrylates) such as [rho]oly(methyl methacrylate), poly(butyl methacrylate), and Poly(2-hydroxy ethyl methacrylate), Poly(vinyl alcohol) Poly(olefins) such as poly(ethylene), [rho]oly(isoprene), halogenated polymers such as Poly(tetrafluoroethylene)—and derivatives and copolymers such as those commonly sold as Teflon(R) products, Poly(vinylidine fluoride), Poly(vinyl acetate), Poly(vinyl pyrrolidone), Poly(acrylic acid), Polyacrylamide, Poly(ethylene-co-vinyl acetate), Poly(ethylene glycol), Poly(propylene glycol), Poly(methacrylic acid); etc.

Examples of polymers that may be used in the present invention include, but are not limited to polycarboxylic acids, cellulosic polymers, proteins, polypeptides, polyvinylpyrrolidone, maleic anhydride polymers, polyamides, polyvinyl alcohols, polyethylene oxides, glycosaminoglycans, polysaccharides, polyesters, aliphatic polyesters, polyurethanes, polystyrenes, copolymers, silicones, silicone containing polymers, polyalkyl siloxanes, polyorthoesters, polyanhydrides, copolymers of vinyl monomers, polycarbonates, polyethylenes, polypropytenes, polylactic acids, polylactides, polyglycolic acids, polyglycolides, polylactide-co-glycolides, polycaprolactones, poly(e-caprolactone)s, polyhydroxybutyrate valerates, polyacrylamides, polyethers, polyurethane dispersions, polyacrylates, acrylic latex dispersions, polyacrylic acid, polyalkyl methacrylates, polyalkylene-co-vinyl acetates, polyalkylenes, aliphatic polycarbonates polyhydroxyalkanoates, polytetrahalooalkylenes, poly(phosphasones), polytetrahalooalkylenes, poly(phosphasones), and mixtures, combinations, and copolymers thereof.

The polymers of the present invention may be natural or synthetic in origin, including gelatin, chitosan, dextrin, cyclodextrin, Poly(urethanes), Poly(siloxanes) or silicones, Poly(acrylates) such as [rho]oly(methyl methacrylate), poly(butyl methacrylate), and Poly(2-hydroxy ethyl methacrylate), Poly(vinyl alcohol) Poly(olefins) such as poly(ethylene), [rho]oly(isoprene), halogenated polymers such as Poly(tetrafluoroethylene)—and derivatives and copolymers such as those commonly sold as Teflon(R) products, Poly(vinylidine fluoride), Poly(vinyl acetate), Poly(vinyl pyrrolidone), Poly(acrylic acid), Polyacrylamide, Poly(ethylene-co-vinyl acetate), Poly(ethylene glycol), Poly(propylene glycol), Poly(methacrylic acid); etc.

Suitable polymers also include absorbable and/or resorbable polymers including the following, combinations, copolymers and derivatives of the following: Polylactides (PLA), Polyglycolides (PGA), PolyLactide-co-glycolides (PLGA), Polyanhydrides, Polyorthoesters, Poly(N-(2-hydroxypropyl) methacrylamide), Poly(l-aspartamide), including the derivatives DLPLA—poly(dl-lactide); LPLA—poly(l-lactide); PDO—poly(dioxanone); PGA-TMC—poly(glycolide-co-trimethylene carbonate); PGA-LPLA—poly(l-lactide-co-glycolide); PGA-DLPLA—poly(dl-lactide-co-glycolide); LPLA-DLPLA—poly(l-lactide-co-dl-lactide); and PDO-PGA-TMC—poly(glycolide-co-trimethylene carbonate-co-dioxanone), and combinations thereof.

“Copolymer” as used herein refers to a polymer being composed of two or more different monomers. A copolymer may also and/or alternatively refer to random, block, graft, copolymers known to those of skill in the art.

“Biocompatible” as used herein, refers to any material that does not cause injury or death to the animal or induce an adverse reaction in an animal when placed in intimate contact with the animal's tissues. Adverse reactions include for example inflammation, infection, fibrotic tissue formation, cell death, or thrombosis. The terms “biocompatible” and “biocompatibility” when used herein are art-recognized and mean that the referent is neither itself toxic to a host (e.g., an animal or human), nor degrades (if it degrades) at a rate that produces byproducts (e.g., monomeric or oligomeric subunits or other byproducts) at toxic concentrations, causes inflammation or irritation, or induces an immune reaction in the host. It is not necessary that any subject composition have a purity of 100% to be deemed biocompatible. Hence, a subject composition may comprise 99%, 98%, 97%, 96%, 95%, 90% 85%, 80%, 75% or even less of biocompatible agents, e.g., including polymers and other materials and excipients described herein, and still be biocompatible.

To determine whether a polymer or other material is biocompatible, it may be necessary to conduct a toxicity analysis. Such assays are well known in the art. One example of such an assay may be performed with live carcinoma cells, such as GT3TKB tumor cells, in the following manner: the sample is degraded in 1 M NaOH at 37° C. until complete degradation is observed. The solution is then neutralized with 1 M HCl. About 200 microliters of various concentrations of the degraded sample products are placed in 96-well tissue culture plates and seeded with human gastric carcinoma cells (GT3TKB) at 104/well density. The degraded sample products are incubated with the GT3TKB cells for 48 hours. The results of the assay may be plotted as % relative growth vs. concentration of degraded sample in the tissue-culture well. In addition, polymers and formulations of the present invention may also be evaluated by well-known in vivo tests, such as subcutaneous implantations in rats to confirm that they do not cause significant levels of irritation or inflammation at the subcutaneous implantation sites.

The terms “bioabsorbable,” “biodegradable,” “bioerodible,” and “bioresorbable,” are art-recognized synonyms. These terms are used herein interchangeably. Bioabsorbable polymers typically differ from non-bioabsorbable polymers (i.e. durable polymers) in that the former may be absorbed (e.g.; degraded) during use. In certain embodiments, such use involves in vivo use, such as in vivo therapy, and in other certain embodiments, such use involves in vitro use. In general, degradation attributable to biodegradability involves the degradation of a bioabsorbable polymer into its component subunits, or digestion, e.g., by a biochemical process, of the polymer into smaller, non-polymeric subunits. In certain embodiments, biodegradation may occur by enzymatic mediation, degradation in the presence of water (hydrolysis) and/or other chemical species in the body, or both. The bioabsorbabilty of a polymer may be shown in-vitro as described herein or by methods known to one of skill in the art. An in-vitro test for bioabsorbability of a polymer does not require living cells or other biologic materials to show bioabsorption properties (e.g. degradation, digestion). Thus, resorbtion, resorption, absorption, absorbtion, erosion may also be used synonymously with the terms “bioabsorbable,” “biodegradable,” “bioerodible,” and “bioresorbable.” Mechanisms of degradation of a bioabsorbable polymer may include, but are not limited to, bulk degradation, surface erosion, and combinations thereof.

As used herein, the term “biodegradation” encompasses both general types of biodegradation. The degradation rate of a biodegradable polymer often depends in part on a variety of factors, including the chemical identity of the linkage responsible for any degradation, the molecular weight, crystallinity, biostability, and degree of cross-linking of such polymer, the physical characteristics (e.g., shape and size) of the implant, and the mode and location of administration. For example, the greater the molecular weight, the higher the degree of crystallinity, and/or the greater the biostability, the biodegradation of any bioabsorbable polymer is usually slower.

As used herein, the term “durable polymer” refers to a polymer that is not bioabsorbable (and/or is not bioerodable, and/or is not biodegradable, and/or is not bioresorbable) and is, thus biostable. In some embodiments, the device comprises a durable polymer. The polymer may include a cross-linked durable polymer. Example biocompatible durable polymers include, but are not limited to: polyester, aliphatic polyester, polyanhydride, polyethylene, polyorthoester, polyphosphazene, polyurethane, polycarbonate urethane, aliphatic polycarbonate, silicone, a silicone containing polymer, polyolefin, polyamide, polycaprolactam, polyamide, polyvinyl alcohol, acrylic polymer, acrylate, polystyrene, epoxy, polyethers, cellulosics, expanded polytetrafluoroethylene, phosphorylcholine, polyethyleneyerphthalate, polymethylmethavrylate, poly(ethylmethacrylate/n-butylmethacrylate), parylene C, polyethylene-co-vinyl acetate, polyalkyl methacrylates, polyalkylene-co-vinyl acetate, polyalkylene, polyalkyl siloxanes, polyhydroxyalkanoate, polyfluoroalkoxyphasphazine, poly(styrene-b-isobutylene-b-styrene), poly-butyl methacrylate, poly-byta-diene, and blends, combinations, homopolymers, condensation polymers, alternating, block, dendritic, crosslinked, and copolymers thereof. The polymer may include a thermoset material. The polymer may provide strength for the coated implantable medical device. The polymer may provide durability for the coated implantable medical device. The coatings and coating methods provided herein provide substantial protection from these by establishing a multi-layer coating which can be bioabsorbable or durable or a combination thereof, and which can both deliver active agents and provide elasticity and radial strength for the vessel in which it is delivered.

“Therapeutically desirable morphology” as used herein refers to the gross form and structure of the pharmaceutical agent, once deposited on the substrate, so as to provide for optimal conditions of ex vivo storage, in vivo preservation and/or in vivo release. Such optimal conditions may include, but are not limited to increased shelf life, increased in vivo stability, good biocompatibility, good bioavailability or modified release rates. Typically, for the present invention, the desired morphology of a pharmaceutical agent would be crystalline or semi-crystalline or amorphous, although this may vary widely depending on many factors including, but not limited to, the nature of the pharmaceutical agent, the disease to be treated/prevented, the intended storage conditions for the substrate prior to use or the location within the body of any biomedical implant. Preferably at least 10%, 20%, 30%, 40%, 50%, 60%, 70%, 80%, 90% or 100% of the pharmaceutical agent is in crystalline or semi-crystalline form.

“Stabilizing agent” as used herein refers to any substance that maintains or enhances the stability of the biological agent. Ideally these stabilizing agents are classified as Generally Regarded As Safe (GRAS) materials by the US Food and Drug Administration (FDA). Examples of stabilizing agents include, but are not limited to carrier proteins, such as albumin, gelatin, metals or inorganic salts. Pharmaceutically acceptable excipient that may be present can further be found in the relevant literature, for example in the Handbook of Pharmaceutical Additives: An International Guide to More Than 6000 Products by Trade Name, Chemical, Function, and Manufacturer; Michael and Irene Ash (Eds.); Gower Publishing Ltd.; Aldershot, Hampshire, England, 1995.

“Compressed fluid” as used herein refers to a fluid of appreciable density (e.g., >0.2 g/cc) that is a gas at standard temperature and pressure. “Supercritical fluid”, “near-critical fluid”, “near-supercritical fluid”, “critical fluid”, “densified fluid” or “densified gas” as used herein refers to a compressed fluid under conditions wherein the temperature is at least 80% of the critical temperature of the fluid and the pressure is at least 50% of the critical pressure of the fluid, and/or a density of +50% of the critical density of the fluid.

Examples of substances that demonstrate supercritical or near critical behavior suitable for the present invention include, but are not limited to carbon dioxide, isobutylene, ammonia, water, methanol, ethanol, ethane, propane, butane, pentane, dimethyl ether, xenon, sulfur hexafluoride, halogenated and partially halogenated materials such as chlorofluorocarbons, hydrochlorofluorocarbons, hydro fluorocarbons, perfluorocarbons (such as perfluoromethane and perfuoropropane, chloroform, trichloro-fluoromethane, dichloro-difluoromethane, dichloro-tetrafluoroethane) and mixtures thereof. Preferably, the supercritical fluid is hexafluoropropane (FC-236EA), or 1,1,1,2,3,3-hexafluoropropane. Preferably, the supercritical fluid is hexafluoropropane (FC-236EA), or 1,1,1,2,3,3-hexafluoropropane for use in PLGA polymer coatings.

“Sintering” as used herein refers to the process by which parts of the polymer or the entire polymer becomes continuous (e.g., formation of a continuous polymer film). As discussed below, the sintering process is controlled to produce a fully conformal continuous polymer (complete sintering) or to produce regions or domains of continuous coating while producing voids (discontinuities) in the polymer. As well, the sintering process is controlled such that some phase separation is obtained or maintained between polymer different polymers (e.g., polymers A and B) and/or to produce phase separation between discrete polymer particles. Through the sintering process, the adhesions properties of the coating are improved to reduce flaking of detachment of the coating from the substrate during manipulation in use. As described below, in some embodiments, the sintering process is controlled to provide incomplete sintering of the polymer. In embodiments involving incomplete sintering, a polymer is formed with continuous domains, and voids, gaps, cavities, pores, channels or, interstices that provide space for sequestering a therapeutic agent which is released under controlled conditions. Depending on the nature of the polymer, the size of polymer particles and/or other polymer properties, a compressed gas, a densified gas, a near critical fluid or a super-critical fluid may be employed. In one example, carbon dioxide is used to treat a substrate that has been coated with a polymer and a drug, using dry powder and RESS electrostatic coating processes. In another example, isobutylene is employed in the sintering process. In other examples a mixture of carbon dioxide and isobutylene is employed. In another example, 1,1,2,3,3-hexafluoropropane is employed in the sintering process.

When an amorphous material is heated to a temperature above its glass transition temperature, or when a crystalline material is heated to a temperature above a phase transition temperature, the molecules comprising the material are more mobile, which in turn means that they are more active and thus more prone to reactions such as oxidation. However, when an amorphous material is maintained at a temperature below its glass transition temperature, its molecules are substantially immobilized and thus less prone to reactions. Likewise, when a crystalline material is maintained at a temperature below its phase transition temperature, its molecules are substantially immobilized and thus less prone to reactions. Accordingly, processing drug components at mild conditions, such as the deposition and sintering conditions described herein, minimizes cross-reactions and degradation of the drug component. One type of reaction that is minimized by the processes of the invention relates to the ability to avoid conventional solvents which in turn minimizes-oxidation of drug, whether in amorphous, semi-crystalline, or crystalline form, by reducing exposure thereof to free radicals, residual solvents, protic materials, polar-protic materials, oxidation initiators, and autoxidation initiators.

“Rapid Expansion of Supercritical Solutions” or “RESS” as used herein involves the dissolution of a polymer into a compressed fluid, typically a supercritical fluid, followed by rapid expansion into a chamber at lower pressure, typically near atmospheric conditions. The rapid expansion of the supercritical fluid solution through a small opening, with its accompanying decrease in density, reduces the dissolution capacity of the fluid and results in the nucleation and growth of polymer particles. The atmosphere of the chamber is maintained in an electrically neutral state by maintaining an isolating “cloud” of gas in the chamber. Carbon dioxide, nitrogen, argon, helium, or other appropriate gas is employed to prevent electrical charge is transferred from the substrate to the surrounding environment.

“Bulk properties” properties of a coating including a pharmaceutical or a biological agent that can be enhanced through the methods of the invention include for example: adhesion, smoothness, conformality, thickness, and compositional mixing.

“Electrostatically charged” or “electrical potential” or “electrostatic capture” or “e-” as used herein refers to the collection of the spray-produced particles upon a substrate that has a different electrostatic potential than the sprayed particles. Thus, the substrate is at an attractive electronic potential with respect to the particles exiting, which results in the capture of the particles upon the substrate. i.e. the substrate and particles are oppositely charged, and the particles transport through the gaseous medium of the capture vessel onto the surface of the substrate is enhanced via electrostatic attraction. This may be achieved by charging the particles and grounding the substrate or conversely charging the substrate and grounding the particles, by charging the particles at one potential (e.g. negative charge) and charging the substrate at an opposite potential (e.g. positive charge), or by some other process, which would be easily envisaged by one of skill in the art of electrostatic capture.

“Intimate mixture” as used herein, refers to two or more materials, compounds, or substances that are uniformly distributed or dispersed together.

“Layer” as used herein refers to a material covering a surface or forming an overlying part or segment. Two different layers may have overlapping portions whereby material from one layer may be in contact with material from another layer. Contact between materials of different layers can be measured by determining a distance between the materials. For example, Raman spectroscopy may be employed in identifying materials from two layers present in close proximity to each other.

While layers defined by uniform thickness and/or regular shape are contemplated herein, several embodiments described below relate to layers having varying thickness and/or irregular shape. Material of one layer may extend into the space largely occupied by material of another layer. For example, in a coating having three layers formed in sequence as a first polymer layer, a pharmaceutical agent layer and a second polymer layer, material from the second polymer layer which is deposited last in this sequence may extend into the space largely occupied by material of the pharmaceutical agent layer whereby material from the second polymer layer may have contact with material from the pharmaceutical layer. It is also contemplated that material from the second polymer layer may extend through the entire layer largely occupied by pharmaceutical agent and contact material from the first polymer layer.

It should be noted however that contact between material from the second polymer layer (or the first polymer layer) and material from the pharmaceutical agent layer (e.g.; a pharmaceutical agent crystal particle or a portion thereof) does not necessarily imply formation of a mixture between the material from the first or second polymer layers and material from the pharmaceutical agent layer. In some embodiments, a layer may be defined by the physical three-dimensional space occupied by crystalline particles of a pharmaceutical agent (and/or biological agent). It is contemplated that such layer may or may not be continuous as physical space occupied by the crystal particles of pharmaceutical agents may be interrupted, for example, by polymer material from an adjacent polymer layer. An adjacent polymer layer may be a layer that is in physical proximity to be pharmaceutical agent particles in the pharmaceutical agent layer. Similarly, an adjacent layer may be the layer formed in a process step right before or right after the process step in which pharmaceutical agent particles are deposited to form the pharmaceutical agent layer.

As described below, material deposition and layer formation provided herein are advantageous in that the pharmaceutical agent remains largely in crystalline form during the entire process. While the polymer particles and the pharmaceutical agent particles may be in contact, the layer formation process is controlled to avoid formation of a mixture between the pharmaceutical agent particles the polymer particles during formation of a coated device.

“Laminate coating” as used herein refers to a coating made up of two or more layers of material. Means for creating a laminate coating as described herein (e.g.; a laminate coating comprising bioabsorbable polymer(s) and pharmaceutical agent) may include coating the stent with drug and polymer as described herein (e-RESS, e-DPC, compressed-gas sintering). The process comprises performing multiple and sequential coating steps (with sintering steps for polymer materials) wherein different materials may be deposited in each step, thus creating a laminated structure with a multitude of layers (at least 2 layers) including polymer layers and pharmaceutical agent layers to build the final device (e.g.; laminate coated stent).

The coating methods provided herein may be calibrated to provide a coating bias whereby the mount of polymer and pharmaceutical agent deposited in the abluminal surface of the stent (exterior surface of the stent) is greater than the amount of pharmaceutical agent and amount of polymer deposited on the luminal surface of the stent (interior surface of the stent). The resulting configuration may be desirable to provide preferential elution of the drug toward the vessel wall (luminal surface of the stent) where the therapeutic effect of anti-restenosis is desired, without providing the same antiproliferative drug(s) on the abluminal surface, where they may retard healing, which in turn is suspected to be a cause of late-stage safety problems with current DESs.

As well, the methods described herein provide a device wherein the coating on the stent is biased in favor of increased coating at the ends of the stent. For example, a stent having three portions along the length of the stent (e.g.; a central portion flanked by two end portions) may have end portions coated with increased amounts of pharmaceutical agent and/or polymer compared to the central portion.

The present invention provides numerous advantages. The invention is advantageous in that it allows for employing a platform combining layer formation methods based on compressed fluid technologies; electrostatic capture and sintering methods. The platform results in drug eluting stents having enhanced therapeutic and mechanical properties. The invention is particularly advantageous in that it employs optimized laminate polymer technology. In particular, the present invention allows the formation of discrete layers of specific drug platforms. As indicated above, the shape of a discrete layer of crystal particles may be irregular, including interruptions of said layer by material from another layer (polymer layer) positioned in space between crystalline particles of pharmaceutical agent.

Conventional processes for spray coating stents require that drug and polymer be dissolved in solvent or mutual solvent before spray coating can occur. The platform provided herein the drugs and polymers are coated on the stent framework in discrete steps, which can be carried out simultaneously or alternately. This allows discrete deposition of the active agent (e.g., a drug) within a polymer thereby allowing the placement of more than one drug on a single medical device with or without an intervening polymer layer. For example, the present platform provides a dual drug eluting stent.

Some of the advantages provided by the subject invention include employing compressed fluids (e.g., supercritical fluids, for example E-RESS based methods); solvent free deposition methodology; a platform that allows processing at lower temperatures thereby preserving the qualities of the active agent and the polymer; the ability to incorporate two, three or more drugs while minimizing deleterious effects from direct interactions between the various drugs and/or their excipients during the fabrication and/or storage of the drug eluting stents; a dry deposition; enhanced adhesion and mechanical properties of the layers on the stent framework; precision deposition and rapid batch processing; and ability to form intricate structures.

In one embodiment, the present invention provides a multi-drug delivery platform which produces strong, resilient and flexible drug eluting stents including an anti-restenosis drug (e.g., a limus or taxol) and anti-thrombosis drug (e.g., heparin or an analog thereof) and well characterized bioabsorbable polymers. The drug eluting stents provided herein minimize potential for thrombosis, in part, by reducing or totally eliminating thrombogenic polymers and reducing or totally eliminating residual drugs that could inhibit healing.

The platform provides optimized delivery of multiple drug therapies for example for early stage treatment (restenosis) and late-stage (thrombosis).

The platform also provides an adherent coating which enables access through tortuous lesions without the risk of the coating being compromised.

Another advantage of the present platform is the ability to provide highly desirable eluting profiles.

Advantages of the invention include the ability to reduce or completely eliminate potentially thrombogenic polymers as well as possibly residual drugs that may inhibit long term healing. As well, the invention provides advantageous stents having optimized strength and resilience if coatings which in turn allows access to complex lesions and reduces or completely eliminates delamination. Laminated layers of bioabsorbable polymers allow controlled elution of one or more drugs.

The platform provided herein reduces or completely eliminates shortcoming that have been associated with conventional drug eluting stents. For example, the platform provided herein allows for much better tuning of the period of time for the active agent to elute and the period of time necessary for the polymer to resorb thereby minimizing thrombosis and other deleterious effects associate with poorly controlled drug release.

The present invention provides several advantages which overcome or attenuate the limitations of current technology for bioabsorbable stents. For example, an inherent limitation of conventional bioabsorbable polymeric materials relates to the difficulty in forming to a strong, flexible, deformable (e.g. balloon deployable) stent with low profile. The polymers generally lack the strength of high-performance metals. The present invention overcomes these limitations by creating a laminate structure in the essentially polymeric stent. Without wishing to be bound by any specific theory or analogy, the increased strength provided by the stents of the invention can be understood by comparing the strength of plywood vs. the strength of a thin sheet of wood.

Embodiments of the invention involving a thin metallic stent-framework provide advantages including the ability to overcome the inherent elasticity of most polymers. It is generally difficult to obtain a high rate (e.g., 100%) of plastic deformation in polymers (compared to elastic deformation where the materials have some ‘spring back’ to the original shape). Again, without wishing to be bound by any theory, the central metal stent framework (that would be too small and weak to serve as a stent itself) would act like wires inside of a plastic, deformable stent, basically overcoming any ‘elastic memory’ of the polymer.

Another advantage of the present invention is the ability to create a stent with a controlled (dialed-in) drug-elution profile. Via the ability to have different materials in each layer of the laminate structure and the ability to control the location of drug(s) independently in these layers, the method enables a stent that could release drugs at very specific elution profiles, programmed sequential and/or parallel elution profiles. Also, the present invention allows controlled elution of one drug without affecting the elution of a second drug (or different doses of the same drug).

Provided herein is a device comprising a stent; and a coating on the stent; wherein the coating comprises at least one bioabsorbable polymer and at least one active agent; wherein the active agent is present in crystalline form on at least one region of an outer surface of the coating opposite the stent and wherein 50% or less of the total amount of active agent in the coating is released after 24 hours in vitro elution.

In some embodiments, in vitro elution is carried out in a 1:1 spectroscopic grade ethanol (95%)/phosphate buffer saline at pH 7.4 and 37° C.; wherein the amount of active agent released is determined by measuring UV absorption. In some embodiments, UV absorption is detected at 278 nm by a diode array spectrometer.

In some embodiments, in vitro elution testing, and/or any other test method described herein is performed following the final sintering step. In some embodiments, in vitro elution testing, and/or any other test method described herein is performed prior to crimping the stent to a balloon catheter. In some embodiments, in vitro elution testing, and/or any other test method described herein is performed following sterilization. In some embodiments in vitro elution testing, and/or any other test method described herein is performed following crimping the stent to a balloon catheter. In some embodiments, in vitro elution testing, and/or any other test method described herein is performed following expansion of the stent to nominal pressure of the balloon onto which the stent has been crimped. In some embodiments, in vitro elution testing, and/or any other test method described herein is performed following expansion of the stent to the rated burst pressure of the balloon to which the stent has been crimped.

In some embodiments, presence of active agent on at least a region of the surface of the coating is determined by cluster secondary ion mass spectrometry (cluster SIMS). In some embodiments, presence of active agent on at least a region of the surface of the coating is determined by generating cluster secondary ion mass spectrometry (cluster SIMS) depth profiles. In some embodiments, presence of active agent on at least a region of the surface of the coating is determined by time of flight secondary ion mass spectrometry (TOF-SIMS). In some embodiments, presence of active agent on at least a region of the surface of the coating is determined by atomic force microscopy (AFM). In some embodiments, presence of active agent on at least a region of the surface of the coating is determined by X-ray spectroscopy. In some embodiments, presence of active agent on at least a region of the surface of the coating is determined by electronic microscopy. In some embodiments, presence of active agent on at least a region of the surface of the coating is determined by Raman spectroscopy.

In some embodiments, between 25% and 45% of the total amount of active agent in the coating is released after 24 hours in vitro elution in a 1:1 spectroscopic grade ethanol (95%)/phosphate buffer saline at pH 7.4 and 37° C.; wherein the amount of the active agent released is determined by measuring UV absorption at 278 nm by a diode array spectrometer.

In some embodiments, the active agent is at least 50% crystalline. In some embodiments, the active agent is at least 75% crystalline. In some embodiments, the active agent is at least 90% crystalline.

In some embodiments, the polymer comprises a PLGA copolymer. In some embodiments, the coating comprises a first PLGA copolymer with a ratio of about 40:60 to about 60:40 and a second PLGA copolymer with a ratio of about 60:40 to about 90:10. In some embodiments, the coating comprises a first PLGA copolymer having a molecular weight of about 10 kD (weight average molecular weight) and a second polymer is a PLGA copolymer having a molecular weight of about 19 kD (weight average molecular weight). In some embodiments, the coating comprises a PLGA copolymer having a number average molecular weight of between about 9.5 kD and about 25 kD. In some embodiments, the coating comprises a PLGA copolymer having a number average molecular weight of between about 14.5 kD and about 15 kD. As used herein, the term “about,” when referring to a copolymer ratio, means variations of any of 0.5%, 1%, 2%, 5%, 10%, 15%, 20%, 25%, 30%, and 50%, depending on the embodiment. For example, a copolymer ratio of 40:60 having a variation of 10% ranges from 35:65 to 45:55, which is a range of 10% of the total (100) about the target. As used herein, the term “about” when referring to a polymer molecular weight means variations of any of 0.5%, 1%, 2%, 5%, 10%, 15%, 20%, 25%, 30%, and 50%, depending on the embodiment. For example, a polymer molecular weight of 10 kD (weight average molecular weight) having a variation of 10% ranges from 9 kD to 11 kD, which is a range of 10% of the target 10 kD (weight average molecular weight) on either side of the target 10 kD (weight average molecular weight).

In some embodiments, the bioabsorbable polymer is selected from the group PLGA, PGA poly(glycolide), LPLA poly(l-lactide), DLPLA poly(dl-lactide), PCL poly(e-caprolactone) PDO, poly(dioxolane) PGA-TMC, 85/15 DLPLG p(dl-lactide-co-glycolide), 75/25 DLPLG, 65/35 DLPLG, 50/50 DLPLG, TMC poly(trimethylcarbonate), poly(anhydrides) such as p(CPP:SA) poly(1,3-bis-p-(carboxyphenoxy)propane-co-sebacic acid).

In some embodiments, the stent is formed of stainless steel material. In some embodiments, the stent is formed of a material comprising a cobalt chromium alloy. In some embodiments, the stent is formed from a material comprising the following percentages by weight: about 0.05 to about 0.15 C, about 1.00 to about 2.00 Mn, about 0.04 Si, about 0.03 P, about 0.3 S, about 19.0 to about 21.0 Cr, about 9.0 to about 11.0 Ni, about 14.0 to about 16.00 W, about 3.0 Fe, and Bal. Co. In some embodiments, the stent is formed from a material comprising at most the following percentages by weight: about 0.025 C, about 0.15 Mn, about 0.15 Si, about 0.015 P, about 0.01 S, about 19.0 to about 21.0 Cr, about 33 to about 37 Ni, about 9.0 to about 10.5 Mo, about 1.0 Fe, about 1.0 Ti, and Bal. Co. In some embodiments, the stent is formed from a material comprising L605 alloy. In some embodiments, the stent is formed from a material comprising MP35N alloy. In some embodiments, the stent is formed from a material comprising the following percentages by weight: about 35 Ni, about 35 Cr, about 20 Co, and about 10 Mo. In some embodiments, the stent is formed from a material comprising a cobalt chromium nickel alloy. In some embodiments, the stent is formed from a material comprising Elgiloy®/Phynox®. In some embodiments, the stent is formed from a material comprising the following percentages by weight: about 39 to about 41 Co, about 19 to about 21 Cr, about 14 to about 16 Ni, about 6 to about 8 Mo, and Balance Fe. In some embodiments, the stent is formed of a material comprising a platinum chromium alloy. In some embodiments, the stent is formed of an alloy as described in U.S. Pat. No. 7,329,383 incorporated in its entirety herein by reference. In some embodiments, the stent is formed of an alloy as described in U.S. patent application Ser. No. 11/780,060 incorporated in its entirety herein by reference. In some embodiments, the stent may be formed of a material comprising stainless steel, 316L stainless steel, BioDur® 108 (UNS S29108), 304L stainless steel, and an alloy including stainless steel and 5-60% by weight of one or more radiopaque elements such as Pt, IR, Au, W, PERSS® as described in U.S. Publication No. 2003/001830 incorporated in its entirety herein by reference, U.S. Publication No. 2002/0144757 incorporated in its entirety herein by reference, and U.S. Publication No. 2003/0077200 incorporated in its entirety herein by reference, nitinol, a nickel-titanium alloy, cobalt alloys, Elgiloy®, L605 alloys, MP35N alloys, titanium, titanium alloys, Ti-6Al-4V, Ti-50Ta, Ti-10Ir, platinum, platinum alloys, niobium, niobium alloys, Nb-1Zr, Co-28Cr-6Mo, tantalum, and tantalum alloys. Other examples of materials are described in U.S. Publication No. 2005/0070990 incorporated in its entirety herein by reference, and U.S. Publication No. 2006/0153729 incorporated in its entirety herein by reference. Other materials include elastic biocompatible metal such as superelastic or pseudo-elastic metal alloys, as described, for example in Schetsky, L. McDonald, “Shape Memory Alloys”, Encyclopedia of Chemical Technology (3d Ed), John Wiley & Sons 1982, vol. 20 pp. 726-736 incorporated herein by reference, and U.S. Publication No. 2004/0143317 incorporated in its entirety herein by reference. As used herein, the term “about,” when referring to a weight percentage of stent material, means variations of any of 0.5%, 1%, 2%, 5%, 10%, 15%, 20%, 25%, 30%, and 50% of the total weight percent (i.e. 100%) on either side (+/−) of the weight percentage, depending on the embodiment. For example, a weight percentage of stent material of 3.0 Fe having a variation of 1% ranges from 2.0 to 4.0, which is a range of 1% of the total (100) on either side of the target 3.0.

In some embodiments, the stent has a thickness of from about 50% to about 90% of a total thickness of the device. In some embodiments, the device has a thickness of from about 20 μm to about 500 μm. In some embodiments, the stent has a thickness of from about 50 μm to about 80 μm. In some embodiments, the coating has a total thickness of from about 5 μm to about 50 μm. The coating can be conformal around the struts, isolated on the abluminal side, patterned, or otherwise optimized for the target tissue.

In some embodiments, the device has an active agent content of from about 5 μg to about 500 μg. In some embodiments, the device has an active agent content of from about 100 μg to about 160 μg. As used herein, the term “about” when referring to a device thickness or coating thickness means variations of any of 0.5%, 1%, 2%, 5%, 10%, 15%, 20%, 25%, 30%, and 50%, depending on the embodiment. For non-limiting example, a device thickness of 20 μm having a variation of 10% ranges from 18 μm to 22 μm, which is a range of 10% on either side of the target 20 μm. For non-limiting example, a coating thickness of 100 μm having a variation of 10% ranges from 90 μm to 110 μm, which is a range of 10% on either side of the target 100 μm. As used herein, the term “about” when referring to an active agent (or pharmaceutical agent) content means variations of any of 0.5%, 1%, 2%, 5%, 10%, 15%, 20%, 25%, 30%, and 50%, depending on the embodiment. For non-limiting example, an active agent content of 120 μg having a variation of 10% ranges from 108 μg to 132 μg, which is a range of 10% on either side of the target 120 μg.

In some embodiments, the active agent is selected from rapamycin, a prodrug, a derivative, an analog, a hydrate, an ester, and a salt thereof. In some embodiments, the active agent is selected from one or more of sirolimus, everolimus, zotarolimus and biolimus. In some embodiments, the active agent comprises a macrolide immunosuppressive (limus) drug. In some embodiments, the macrolide immunosuppressive drug comprises one or more of rapamycin, biolimus (biolimus A9), 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′:E,4′S)-40-O-(4′,5′-Dihydroxypent-2′-en-1′-yl)-rapamycin, 40-O-(2-Hydroxy)ethoxycarbonylmethyl-rapamycin, 40-O-(3-Hydroxyl)propyl-rapamycin, 40-O-(6-Hydroxyl)hexyl-rapamycin, 40-O-[2-(2-Hydroxyl)ethoxy]ethyl-rapamycin, 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin 40-O-(2-Nicotinoyloxy)ethyl-rapamycin, 40-O-[2-(N-Morpholino)acetoxy]ethyl-rapamycin, 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxyl)ethyl-rapamycin, 28-O-Methyl-rapamycin, 40-O-(2-Aminoethyl)-rapamycin, 40-O-(2-Acetaminoethyl)-rapamycin, 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl)rapamycin (tacrolimus), 42-[3-hydroxy-2-(hydroxymethyl)-2-methylpropanoate]rapamycin (temsirolimus), (42S)-42-Deoxy-42-(1H-tetrazol-1-yl)-rapamycin (zotarolimus), and salts, derivatives, isomers, racemates, diastereoisomers, prodrugs, hydrate, ester, or analogs thereof.

In some embodiments, the pharmaceutical agent is, at least in part, crystalline. As used herein, the term crystalline may include any number of the possible polymorphs of the crystalline form of the pharmaceutical agent, including for non-limiting example a single polymorph of the pharmaceutical agent, or a plurality of polymorphs of the pharmaceutical agent. The crystalline pharmaceutical agent (which may include a semi-crystalline form of the pharmaceutical agent, depending on the embodiment) may comprise a single polymorph of the possible polymorphs of the pharmaceutical agent. The crystalline pharmaceutical agent (which may include a semi-crystalline form of the pharmaceutical agent, depending on the embodiment) may comprise a plurality of polymorphs of the possible polymorphs of the crystalline pharmaceutical agent. The polymorph, in some embodiments, is a packing polymorph, which exists as a result of difference in crystal packing as compared to another polymorph of the same crystalline pharmaceutical agent. The polymorph, in some embodiments, is a conformational polymorph, which is conformer of another polymorph of the same crystalline pharmaceutical agent. The polymorph, in some embodiments, is a pseudopolymorph. The polymorph, in some embodiments, is any type of polymorph—that is, the type of polymorph is not limited to only a packing polymorph, conformational polymorph, and/or a pseudopolymorph. When referring to a particular pharmaceutical agent herein which is at least in part crystalline, it is understood that any of the possible polymorphs of the pharmaceutical agent are contemplated.

Provided herein is a device comprising a stent; and a coating on the stent; wherein the coating comprises at least one polymer and at least one active agent; wherein the active agent is present in crystalline form on at least one region of an outer surface of the coating opposite the stent and wherein between 25% and 50% of the total amount of active agent in the coating is released after 24 hours in vitro elution.

In some embodiments, the polymer comprises a durable polymer. In some embodiments, the polymer comprises a cross-linked durable polymer. Example biocompatible durable polymers include, but are not limited to: polyester, aliphatic polyester, polyanhydride, polyethylene, polyorthoester, polyphosphazene, polyurethane, polycarbonate urethane, aliphatic polycarbonate, silicone, a silicone containing polymer, polyolefin, polyamide, polycaprolactam, polyamide, polyvinyl alcohol, acrylic polymer, acrylate, polystyrene, epoxy, polyethers, cellulosics, expanded polytetrafluoroethylene, phosphorylcholine, polyethyleneyerphthalate, polymethylmethavrylate, poly(ethylmethacrylate/n-butylmethacrylate), parylene C, polyethylene-co-vinyl acetate, polyalkyl methacrylates, polyalkylene-co-vinyl acetate, polyalkylene, polyalkyl siloxanes, polyhydroxyalkanoate, polyfluoroalkoxyphasphazine, poly(styrene-b-isobutylene-b-styrene), poly-butyl methacrylate, poly-byta-diene, and blends, combinations, homopolymers, condensation polymers, alternating, block, dendritic, crosslinked, and copolymers thereof.

In some embodiments, the polymer comprises is at least one of a fluoropolymer, PVDF-HFP comprising vinylidene fluoride and hexafluoropropylene monomers, PC (phosphorylcholine), Polysulfone, polystyrene-b-isobutylene-b-styrene, PVP (polyvinylpyrrolidone), alkyl methacrylate, vinyl acetate, hydroxyalkyl methacrylate, and alkyl acrylate. In some embodiments, the alkyl methacrylate comprises at least one of methyl methacrylate, ethyl methacrylate, propyl methacrylate, butyl methacrylate, hexyl methacrylate, octyl methacrylate, dodecyl methacrylate, and lauryl methacrylate. In some embodiments, the alkyl acrylate comprises at least one of methyl acrylate, ethyl acrylate, propyl acrylate, butyl acrylate, hexyl acrylate, octyl acrylate, dodecyl acrylates, and lauryl acrylate.

In some embodiments, the coating comprises a plurality of polymers. In some embodiments, the polymers comprise hydrophilic, hydrophobic, and amphiphilic monomers and combinations thereof. In one embodiment, the polymer comprises at least one of a homopolymer, a copolymer and a terpolymer. The homopolymer may comprise a hydrophilic polymer constructed of a hydrophilic monomer selected from the group consisting of poly(vinylpyrrolidone) and poly(hydroxylalkyl methacrylate). The copolymer may comprise comprises a polymer constructed of hydrophilic monomers selected from the group consisting of vinyl acetate, vinylpyrrolidone and hydroxyalkyl methacrylate and hydrophobic monomers selected from the group consisting of alkyl methacrylates including methyl, ethyl, propyl, butyl, hexyl, octyl, dodecyl, and lauryl methacrylate and alkyl acrylates including methyl, ethyl, propyl, butyl, hexyl, octyl, dodecyl, and lauryl acrylate. The terpolymer may comprise a polymer constructed of hydrophilic monomers selected from the group consisting of vinyl acetate and poly(vinylpyrrolidone), and hydrophobic monomers selected from the group consisting of alkyl methacrylates including methyl, ethyl, propyl, butyl, hexyl, octyl, dodecyl, and lauryl methacrylate and alkyl acrylates including methyl, ethyl, propyl, butyl, hexyl, octyl, dodecyl, and lauryl acrylate.

In one embodiment, the polymer comprises three polymers: a terpolymer, a copolymer and a homopolymer. In one such embodiment the terpolymer has the lowest glass transition temperature (Tg), the copolymer has an intermediate Tg and the homopolymer has the highest Tg. In one embodiment the ratio of terpolymer to copolymer to homopolymer is about 40:40:20 to about 88:10:2. In another embodiment, the ratio is about 50:35:15 to about 75:20:5. In one embodiment the ratio is approximately 63:27:10. In such embodiments, the terpolymer has a Tg in the range of about 5° C. to about 25° C., a copolymer has a Tg in the range of about 25° C. to about 40° C. and a homopolymer has a Tg in the range of about 170° C. to about 180° C. In some embodiments, the polymer system comprises a terpolymer (C19) comprising the monomer subunits n-hexyl methacrylate, N-vinylpyrrolidone and vinyl acetate having a Tg of about 10° C. to about 20° C., a copolymer (C10) comprising the monomer subunits n-butyl methacrylate and vinyl acetate having a Tg of about 30° C. to about 35° C. and a homopolymer comprising polyvinylpyrrolidone having a Tg of about 174° C. As used herein, the term “about,” when referring to a polymer ratio, means variations of any of 0.5%, 1%, 2%, 5%, 10%, 15%, 20%, 25%, 30%, and 50%, depending on the embodiment. For non-limiting example, a ratio of 40:40:20 having a variation of 10% around each of the polymers (e.g. the terpolymer may be 35-45%; the copolymer may be 35-45%, and the homopolymer may be 15 to 25% of the total). As used herein, the term “about,” when referring to a Tg, means variations of any of 0.5%, 1%, 2%, 5%, 10%, 15%, 20%, 25%, 30%, and 50%, depending on the embodiment. For non-limiting example, a Tg of 30° C. having a variation of 10% means a range of Tg from 27° C. to 33° C.

Some embodiments comprise about 63% of C19, about 27% of C10 and about 10% of polyvinyl pyrrolidone (PVP). The C10 polymer is comprised of hydrophobic n-butyl methacrylate to provide adequate hydrophobicity to accommodate the active agent and a small amount of vinyl acetate. The C19 polymer is soft relative to the C10 polymer and is synthesized from a mixture of hydrophobic n-hexyl methacrylate and hydrophilic N-vinyl pyrrolidone and vinyl acetate monomers to provide enhanced biocompatibility. Polyvinyl pyrrolidone (PVP) is a medical grade hydrophilic polymer.

In some embodiments, the polymer is not a polymer selected from: PBMA (poly n-butyl methacrylate), Parylene C, and polyethylene-co-vinyl acetate.

In some embodiments, the polymer comprises a bioabsorbable polymer. In some embodiments, the bioabsorbable polymer is selected from the group PLGA, PGA poly(glycolide), LPLA poly(l-lactide), DLPLA poly(dl-lactide), PCL poly(e-caprolactone) PDO, poly(dioxolane) PGA-TMC, 85/15 DLPLG p(dl-lactide-co-glycolide), 75/25 DLPLG, 65/35 DLPLG, 50/50 DLPLG, TMC poly(trimethylcarbonate), poly(anhydrides) such as p(CPP:SA) poly(1,3-bis-p-(carboxyphenoxy)propane-co-sebacic acid).

In some embodiments, in vitro elution is carried out in a 1:1 spectroscopic grade ethanol (95%)/phosphate buffer saline at pH 7.4 and 37° C.; wherein the amount of active agent released is determined by measuring UV absorption.

In some embodiments, the active agent is at least 50% crystalline. In some embodiments, the active agent is at least 75% crystalline. In some embodiments, the active agent is at least 90% crystalline.

In some embodiments, the stent is formed of stainless steel material. In some embodiments, the stent is formed of a material comprising a cobalt chromium alloy. In some embodiments, the stent is formed from a material comprising the following percentages by weight: about 0.05 to about 0.15 C, about 1.00 to about 2.00 Mn, about 0.04 Si, about 0.03 P, about 0.3 S, about 19.0 to about 21.0 Cr, about 9.0 to about 11.0 Ni, about 14.0 to about 16.00 W, about 3.0 Fe, and Bal. Co. In some embodiments, the stent is formed from a material comprising at most the following percentages by weight: about 0.025 C, about 0.15 Mn, about 0.15 Si, about 0.015 P, about 0.01 S, about 19.0 to about 21.0 Cr, about 33 to about 37 Ni, about 9.0 to about 10.5 Mo, about 1.0 Fe, about 1.0 Ti, and Bal. Co. In some embodiments, the stent is formed from a material comprising L605 alloy. In some embodiments, the stent is formed from a material comprising MP35N alloy. In some embodiments, the stent is formed from a material comprising the following percentages by weight: about 35 Ni, about 35 Cr, about 20 Co, and about 10 Mo. In some embodiments, the stent is formed from a material comprising a cobalt chromium nickel alloy. In some embodiments, the stent is formed from a material comprising Elgiloy®/Phynox®. In some embodiments, the stent is formed from a material comprising the following percentages by weight: about 39 to about 41 Co, about 19 to about 21 Cr, about 14 to about 16 Ni, about 6 to about 8 Mo, and Balance Fe. In some embodiments, the stent is formed of a material comprising a platinum chromium alloy. In some embodiments, the stent is formed of an alloy as described in U.S. Pat. No. 7,329,383 incorporated in its entirety herein by reference. In some embodiments, the stent is formed of an alloy as described in U.S. patent application Ser. No. 11/780,060 incorporated in its entirety herein by reference. In some embodiments, the stent may be formed of a material comprising stainless steel, 316L stainless steel, BioDur® 108 (UNS S29108), 304L stainless steel, and an alloy including stainless steel and 5-60% by weight of one or more radiopaque elements such as Pt, IR, Au, W, PERSS® as described in U.S. Publication No. 2003/001830 incorporated in its entirety herein by reference, U.S. Publication No. 2002/0144757 incorporated in its entirety herein by reference, and U.S. Publication No. 2003/0077200 incorporated in its entirety herein by reference, nitinol, a nickel-titanium alloy, cobalt alloys, Elgiloy®, L605 alloys, MP35N alloys, titanium, titanium alloys, Ti-6Al-4V, Ti-50Ta, Ti-10Ir, platinum, platinum alloys, niobium, niobium alloys, Nb-1Zr, Co-28Cr-6Mo, tantalum, and tantalum alloys. Other examples of materials are described in U.S. Publication No. 2005/0070990 incorporated in its entirety herein by reference, and U.S. Publication No. 2006/0153729 incorporated in its entirety herein by reference. Other materials include elastic biocompatible metal such as superelastic or pseudo-elastic metal alloys, as described, for example in Schetsky, L. McDonald, “Shape Memory Alloys”, Encyclopedia of Chemical Technology (3d Ed), John Wiley & Sons 1982, vol. 20 pp. 726-736 incorporated herein by reference, and U.S. Publication No. 2004/0143317 incorporated in its entirety herein by reference.

In some embodiments, the stent has a thickness of from about 50% to about 90% of a total thickness of the device. In some embodiments, the device has a thickness of from about 20 μm to about 500 μm. In some embodiments, the stent has a thickness of from about 50 μm to about 80 μm. In some embodiments, the coating has a total thickness of from about 5 μm to about 50 μm. The coating can be conformal around the struts, isolated on the abluminal side, patterned, or otherwise optimized for the target tissue. As used herein, the term “about” when referring to a device thickness or coating thickness means variations of any of 0.5%, 1%, 2%, 5%, 10%, 15%, 20%, 25%, 30%, and 50%, depending on the embodiment. For non-limiting example, a device thickness of 20 μm having a variation of 10% ranges from 18 μm to 22 μm, which is a range of 10% on either side of the target 20 μm. For non-limiting example, a coating thickness of 100 μm having a variation of 10% ranges from 90 μm to 110 μm, which is a range of 10% on either side of the target 100 μm.

In some embodiments, the device has a pharmaceutical agent content of from about 5 μg to about 500 μg. In some embodiments, the device has a pharmaceutical agent content of from about 100 μg to about 160 μg. As used herein, the term “about” when referring to an active agent content (or pharmaceutical agent content) means variations of any of 0.5%, 1%, 2%, 5%, 10%, 15%, 20%, 25%, 30%, and 50%, depending on the embodiment. For non-limiting example, an active agent (or pharmaceutical agent) content of 120 μg having a variation of 10% ranges from 108 μg to 132 μg, which is a range of 10% on either side of the target 120 μg.

In some embodiments, the active agent is selected from rapamycin, a prodrug, a derivative, an analog, a hydrate, an ester, and a salt thereof. In some embodiments, the active agent comprises a macrolide immunosuppressive (limus) drug. In some embodiments, the macrolide immunosuppressive drug comprises one or more of rapamycin, biolimus (biolimus A9), 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′:E,4′S)-40-O-(4′,5′-Dihydroxypent-2′-en-1′-yl)-rapamycin, 40-O-(2-Hydroxy)ethoxycar-bonylmethyl-rapamycin, 40-O-(3-Hydroxyl)propyl-rapamycin, 40-O-(6-Hydroxyl)hexyl-rapamycin, 40-O-[2-(2-Hydroxyl)ethoxy]ethyl-rapamycin, 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin 40-O-(2-Nicotinoyloxy)ethyl-rapamycin, 40-O-[2-(N-Morpholino)acetoxy]ethyl-rapamycin, 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxyl)ethyl-rapamycin, 28-O-Methyl-rapamycin, 40-O-(2-Aminoethyl)-rapamycin, 40-O-(2-Acetaminoethyl)-rapamycin, 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl)rapamycin (tacrolimus), 42-[3-hydroxy-2-(hydroxymethyl)-2-methylpropanoate]rapamycin (temsirolimus), (42S)-42-Deoxy-42-(1H-tetrazol-1-yl)-rapamycin (zotarolimus), and salts, derivatives, isomers, racemates, diastereoisomers, prodrugs, hydrate, ester, or analogs thereof.

In some embodiments, the pharmaceutical agent is, at least in part, crystalline. As used herein, the term crystalline may include any number of the possible polymorphs of the crystalline form of the pharmaceutical agent, including for non-limiting example a single polymorph of the pharmaceutical agent, or a plurality of polymorphs of the pharmaceutical agent. The crystalline pharmaceutical agent (which may include a semi-crystalline form of the pharmaceutical agent, depending on the embodiment) may comprise a single polymorph of the possible polymorphs of the pharmaceutical agent. The crystalline pharmaceutical agent (which may include a semi-crystalline form of the pharmaceutical agent, depending on the embodiment) may comprise a plurality of polymorphs of the possible polymorphs of the crystalline pharmaceutical agent. The polymorph, in some embodiments, is a packing polymorph, which exists as a result of difference in crystal packing as compared to another polymorph of the same crystalline pharmaceutical agent. The polymorph, in some embodiments, is a conformational polymorph, which is conformer of another polymorph of the same crystalline pharmaceutical agent. The polymorph, in some embodiments, is a pseudopolymorph. The polymorph, in some embodiments, is any type of polymorph—that is, the type of polymorph is not limited to only a packing polymorph, conformational polymorph, and/or a pseudopolymorph. When referring to a particular pharmaceutical agent herein which is at least in part crystalline, it is understood that any of the possible polymorphs of the pharmaceutical agent are contemplated.

Provided herein is a method comprising providing a device comprising a stent and a coating on the stent wherein the coating comprises PLGA and crystalline sirolimus and wherein following implantation into a subject no more than 25% of the struts are malapposed at 4 months, no more than 20% of the struts are malapposed at 6 months, no more than 10% of the struts are malapposed at 8 months, and no more than 5% of the struts are malapposed at 18 months. In some embodiments is a method comprising providing a device comprising a stent and a coating on the stent wherein the coating comprises PLGA and crystalline sirolimus and wherein following implantation into a subject no more than 25% of the struts are malapposed at 4 months. In some embodiments is a method comprising providing a device comprising a stent and a coating on the stent wherein the coating comprises PLGA and crystalline sirolimus and wherein following implantation into a subject no more than 20% of the struts are malapposed at 6 months. In some embodiments is a method comprising providing a device comprising a stent and a coating on the stent wherein the coating comprises PLGA and crystalline sirolimus and wherein following implantation into a subject no more than 10% of the struts are malapposed at 8 months. In some embodiments is a method comprising providing a device comprising a stent and a coating on the stent wherein the coating comprises PLGA and crystalline sirolimus and wherein following implantation into a subject no more than 5% of the struts are malapposed at 18 months.

In some embodiments, a lot of devices which includes the device has an average percent malapposition of stent struts of about 1% at 4 months, about 1% at 6 months, about 0.5% at 8 months, and/or about 0.1% at 18 months. In some embodiments, a lot of devices which includes the device has an average percent malapposition of stent struts of about 1% at 4 months. In some embodiments, a lot of devices which includes the device has an average percent malapposition of stent struts of about 1% at 6 months. In some embodiments, a lot of devices which includes the device has an average percent malapposition of stent struts of about 0.5% at 8 months. In some embodiments, a lot of devices which includes the device has an average percent malapposition of stent struts of about 0.1% at 18 months. In some embodiments, a lot of devices which includes the device has an average percent malapposition of stent struts of less than 1% at 4 months, less than 1% at 6 months, less than 0.5% at 8 months, and/or less than 0.1% at 18 months. In some embodiments, a lot of devices which includes the device has an average percent malapposition of stent struts of less than 1% at 4 months. In some embodiments, a lot of devices which includes the device has an average percent malapposition of stent struts of less than 1% at 6 months. In some embodiments, a lot of devices which includes the device has an average percent malapposition of stent struts of less than 0.5% at 8 months. In some embodiments, a lot of devices which includes the device has an average percent malapposition of stent struts of less than 0.1% at 18 months.

In some embodiments is a method comprising providing a device comprising a stent and a coating on the stent wherein the coating comprises PLGA and crystalline sirolimus and wherein following implantation into a subject there is no more than 50% of the struts are uncovered at 4 months, no more than 40% of the struts are uncovered at 6 months, no more than 15% of the struts are uncovered at 8 months, and no more than 10% of the struts are uncovered at 18 months. In some embodiments is a method comprising providing a device comprising a stent and a coating on the stent wherein the coating comprises PLGA and crystalline sirolimus and wherein following implantation into a subject there is no more than 50% of the struts are uncovered at 4 months. In some embodiments is a method comprising providing a device comprising a stent and a coating on the stent wherein the coating comprises PLGA and crystalline sirolimus and wherein following implantation into a subject there is no more than 40% of the struts are uncovered at 6 months. In some embodiments is a method comprising providing a device comprising a stent and a coating on the stent wherein the coating comprises PLGA and crystalline sirolimus and wherein following implantation into a subject there is no more than 15% of the struts are uncovered at 8 months. In some embodiments is a method comprising providing a device comprising a stent and a coating on the stent wherein the coating comprises PLGA and crystalline sirolimus and wherein following implantation into a subject there is no more than 10% of the struts are uncovered at 18 months. In some embodiments, a lot of devices which includes the device has an average percent of uncovered stent struts of about 10% at 4 months, about 5% at 6 months, about 5% at 8 months, and/or about 1% at 18 months. In some embodiments, a lot of devices which includes the device has an average percent of uncovered stent struts of about 10% at 4 months. In some embodiments, a lot of devices which includes the device has an average percent of uncovered stent struts of about 5% at 6 months. In some embodiments, a lot of devices which includes the device has an average percent of uncovered stent struts of about 5% at 8 months. In some embodiments, a lot of devices which includes the device has an average percent of uncovered stent struts of about 1% at 18 months. In some embodiments, a lot of devices which includes the device has an average percent of uncovered stent struts of less than 11% at 4 months, less than 5% at 6 months, less than 5% at 8 months, and/or less than 1% at 18 months. In some embodiments, a lot of devices which includes the device has an average percent of uncovered stent struts of less than 11% at 4 months. In some embodiments, a lot of devices which includes the device has an average percent of uncovered stent struts of less than 5% at 6 months. In some embodiments, a lot of devices which includes the device has an average percent of uncovered stent struts of less than 5% at 8 months. In some embodiments, a lot of devices which includes the device has an average percent of uncovered stent struts of less than 1% at 18 months.

In some embodiments is a method comprising providing a device comprising a stent and a coating on the stent wherein the coating comprises PLGA and crystalline sirolimus and wherein following implantation into a subject at least 0.02 mm of neointimal thickness exists on the stent on average at 4 months, at least 0.02 mm of neointimal thickness exists on the stent on average at 6 months, at least 0.06 mm of neointimal thickness exists on the stent on average at 8 months. In some embodiments is a method comprising providing a device comprising a stent and a coating on the stent wherein the coating comprises PLGA and crystalline sirolimus and wherein following implantation into a subject at least 0.02 mm of neointimal thickness exists on the stent on average at 4 months. In some embodiments is a method comprising providing a device comprising a stent and a coating on the stent wherein the coating comprises PLGA and crystalline sirolimus and wherein following implantation into a subject at least 0.05 mm of neointimal thickness exists on the stent on average at 4 months. In some embodiments is a method comprising providing a device comprising a stent and a coating on the stent wherein the coating comprises PLGA and crystalline sirolimus and wherein following implantation into a subject at least 0.02 mm of neointimal thickness exists on the stent on average at 6 months. In some embodiments is a method comprising providing a device comprising a stent and a coating on the stent wherein the coating comprises PLGA and crystalline sirolimus and wherein following implantation into a subject at least 0.075 mm of neointimal thickness exists on the stent on average at 6 months. In some embodiments is a method comprising providing a device comprising a stent and a coating on the stent wherein the coating comprises PLGA and crystalline sirolimus and wherein following implantation into a subject at least 0.06 mm of neointimal thickness exists on the stent on average at 8 months. In some embodiments is a method comprising providing a device comprising a stent and a coating on the stent wherein the coating comprises PLGA and crystalline sirolimus and wherein following implantation into a subject at least 0.1 mm of neointimal thickness exists on the stent on average at 8 months.

In some embodiments, at most 10% of the stent struts are covered by fibrin at 4 months following implantation, at most 4% of the stent struts are covered by fibrin at 6 months following implantation, or at most 1% of the stent struts are covered by fibrin at 8 months following implantation. In some embodiments, at most 10% of the stent struts are covered by fibrin at 4 months following implantation. In some embodiments, at most 4% of the stent struts are covered by fibrin at 6 months following implantation. In some embodiments, at most 1% of the stent struts are covered by fibrin at 8 months following implantation. In some embodiments, a MACE rate of a lot of devices which includes the device is at most 5%, between 1% and 5%, less than 5%, less than 4%, between 3% and 5%, or less than 3.5%. In some embodiments, a MACE rate of a lot of devices which includes the device is at most 5%. In some embodiments, a MACE rate of a lot of devices which includes the device is between 1% and 5%. In some embodiments, a MACE rate of a lot of devices which includes the device is less than 5%. In some embodiments, a MACE rate of a lot of devices which includes the device is less than 3.5%.

In some embodiments is a method comprising providing a device comprising a stent and a coating on the stent wherein the coating comprises PLGA and crystalline sirolimus and wherein following implantation into a subject the late lumen loss is less than 0.55 mm, less than 0.50 mm, less than 0.45 mm, less than 0.40 mm, less than 0.35 mm, or less than 0.30 mm at 9 months following implantation. In some embodiments is a method comprising providing a device comprising a stent and a coating on the stent wherein the coating comprises PLGA and crystalline sirolimus and wherein following implantation into a subject the late lumen loss is less than 0.55 mm at 9 months following implantation. In some embodiments is a method comprising providing a device comprising a stent and a coating on the stent wherein the coating comprises PLGA and crystalline sirolimus and wherein following implantation into a subject the late lumen loss is less than 0.50 mm at 9 months following implantation. In some embodiments is a method comprising providing a device comprising a stent and a coating on the stent wherein the coating comprises PLGA and crystalline sirolimus and wherein following implantation into a subject the late lumen loss is less than 0.45 mm at 9 months following implantation. In some embodiments is a method comprising providing a device comprising a stent and a coating on the stent wherein the coating comprises PLGA and crystalline sirolimus and wherein following implantation into a subject the late lumen loss is less than 0.40 mm at 9 months following implantation. In some embodiments is a method comprising providing a device comprising a stent and a coating on the stent wherein the coating comprises PLGA and crystalline sirolimus and wherein following implantation into a subject the late lumen loss is less than 0.35 mm at 9 months following implantation. In some embodiments is a method comprising providing a device comprising a stent and a coating on the stent wherein the coating comprises PLGA and crystalline sirolimus and wherein following implantation into a subject the late lumen loss is less than 0.30 mm at 9 months following implantation. In some embodiments is a method comprising providing a device comprising a stent and a coating on the stent wherein the coating comprises PLGA and crystalline sirolimus and wherein following implantation into a subject the late lumen loss is less than 0.25 mm at 9 months following implantation. In some embodiments is a method comprising providing a device comprising a stent and a coating on the stent wherein the coating comprises PLGA and crystalline sirolimus and wherein following implantation into a subject the late lumen loss is less than 0.20 mm at 9 months following implantation.

In some embodiments is a method comprising providing a device comprising a stent and a coating on the stent wherein the coating comprises PLGA and crystalline sirolimus and wherein following implantation into a subject the target lesion revascularization (TLR) is less than 1.5%, or less than 1.0% at 9 months following implantation. In some embodiments is a method comprising providing a device comprising a stent and a coating on the stent wherein the coating comprises PLGA and crystalline sirolimus and wherein following implantation into a subject the target lesion revascularization (TLR) is less than 1.5% at 9 months following implantation. In some embodiments is a method comprising providing a device comprising a stent and a coating on the stent wherein the coating comprises PLGA and crystalline sirolimus and wherein following implantation into a subject the target lesion revascularization (TLR) is less than 1.0% at 9 months following implantation.

In some embodiments is a method comprising providing a device comprising a stent and a coating on the stent wherein the coating comprises PLGA and crystalline sirolimus and wherein following implantation into a subject the MACE rate is less than 6.5%, less than 6.0%, less than 5.5%, less than 5.0%, less than 4.5%, or less than 4.0% at 9 months following implantation. In some embodiments is a method comprising providing a device comprising a stent and a coating on the stent wherein the coating comprises PLGA and crystalline sirolimus and wherein following implantation into a subject the MACE rate is less than 6.5% at 9 months following implantation. In some embodiments is a method comprising providing a device comprising a stent and a coating on the stent wherein the coating comprises PLGA and crystalline sirolimus and wherein following implantation into a subject the MACE rate is less than 6.0% at 9 months following implantation. In some embodiments is a method comprising providing a device comprising a stent and a coating on the stent wherein the coating comprises PLGA and crystalline sirolimus and wherein following implantation into a subject the MACE rate is less than 5.5% at 9 months following implantation. In some embodiments is a method comprising providing a device comprising a stent and a coating on the stent wherein the coating comprises PLGA and crystalline sirolimus and wherein following implantation into a subject the MACE rate is less than 5.0% at 9 months following implantation. In some embodiments is a method comprising providing a device comprising a stent and a coating on the stent wherein the coating comprises PLGA and crystalline sirolimus and wherein following implantation into a subject the MACE rate is less than 4.5% at 9 months following implantation. In some embodiments is a method comprising providing a device comprising a stent and a coating on the stent wherein the coating comprises PLGA and crystalline sirolimus and wherein following implantation into a subject the MACE rate is less than 4.0% at 9 months following implantation. In some embodiments is a method comprising providing a device comprising a stent and a coating on the stent wherein the coating comprises PLGA and crystalline sirolimus and wherein following implantation into a subject the MACE rate is less than 3.5% at 9 months following implantation. In some embodiments is a method comprising providing a device comprising a stent and a coating on the stent wherein the coating comprises PLGA and crystalline sirolimus and wherein following implantation into a subject the MACE rate is less than 2.5% at 9 months following implantation.

Provided herein is a device comprising a stent and a coating on the stent wherein the coating comprises PLGA and crystalline sirolimus and wherein following implantation into a subject no more than 25% of the struts are malapposed at 4 months, no more than 20% of the struts are malapposed at 6 months, no more than 10% of the struts are malapposed at 8 months, and no more than 5% of the struts are malapposed at 18 months. In some embodiments is a device comprising a stent and a coating on the stent wherein the coating comprises PLGA and crystalline sirolimus and wherein following implantation into a subject no more than 25% of the struts are malapposed at 4 months. In some embodiments is a device comprising a stent and a coating on the stent wherein the coating comprises PLGA and crystalline sirolimus and wherein following implantation into a subject no more than 20% of the struts are malapposed at 6 months. In some embodiments is a device comprising a stent and a coating on the stent wherein the coating comprises PLGA and crystalline sirolimus and wherein following implantation into a subject no more than 10% of the struts are malapposed at 8 months. In some embodiments is a device comprising a stent and a coating on the stent wherein the coating comprises PLGA and crystalline sirolimus and wherein following implantation into a subject no more than 5% of the struts are malapposed at 18 months.

In some embodiments, a lot of devices which includes the device has an average percent malapposition of stent struts of about 1% at 4 months, about 1% at 6 months, about 0.5% at 8 months, and/or about 0.1% at 18 months. In some embodiments, a lot of devices which includes the device has an average percent malapposition of stent struts of about 1% at 4 months. In some embodiments, a lot of devices which includes the device has an average percent malapposition of stent struts of about 1% at 6 months. In some embodiments, a lot of devices which includes the device has an average percent malapposition of stent struts of about 0.5% at 8 months. In some embodiments, a lot of devices which includes the device has an average percent malapposition of stent struts of about 0.1% at 18 months. In some embodiments, a lot of devices which includes the device has an average percent malapposition of stent struts of less than 1% at 4 months, less than 1% at 6 months, less than 0.5% at 8 months, and/or less than 0.1% at 18 months. In some embodiments, a lot of devices which includes the device has an average percent malapposition of stent struts of less than 1% at 4 months. In some embodiments, a lot of devices which includes the device has an average percent malapposition of stent struts of less than 1% at 6 months. In some embodiments, a lot of devices which includes the device has an average percent malapposition of stent struts of less than 0.5% at 8 months. In some embodiments, a lot of devices which includes the device has an average percent malapposition of stent struts of less than 0.1% at 18 months.

In some embodiments is a device comprising a stent and a coating on the stent wherein the coating comprises PLGA and crystalline sirolimus and wherein following implantation into a subject there is no more than 50% of the struts are uncovered at 4 months, no more than 40% of the struts are uncovered at 6 months, no more than 15% of the struts are uncovered at 8 months, and no more than 10% of the struts are uncovered at 18 months. In some embodiments is a device comprising a stent and a coating on the stent wherein the coating comprises PLGA and crystalline sirolimus and wherein following implantation into a subject there is no more than 50% of the struts are uncovered at 4 months. In some embodiments is a device comprising a stent and a coating on the stent wherein the coating comprises PLGA and crystalline sirolimus and wherein following implantation into a subject there is no more than 40% of the struts are uncovered at 6 months. In some embodiments is a device comprising a stent and a coating on the stent wherein the coating comprises PLGA and crystalline sirolimus and wherein following implantation into a subject there is no more than 15% of the struts are uncovered at 8 months. In some embodiments is a device comprising a stent and a coating on the stent wherein the coating comprises PLGA and crystalline sirolimus and wherein following implantation into a subject there is no more than 10% of the struts are uncovered at 18 months. In some embodiments, a lot of devices which includes the device has an average percent of uncovered stent struts of about 10% at 4 months, about 5% at 6 months, about 5% at 8 months, and/or about 1% at 18 months. In some embodiments, a lot of devices which includes the device has an average percent of uncovered stent struts of about 10% at 4 months. In some embodiments, a lot of devices which includes the device has an average percent of uncovered stent struts of about 5% at 6 months. In some embodiments, a lot of devices which includes the device has an average percent of uncovered stent struts of about 5% at 8 months. In some embodiments, a lot of devices which includes the device has an average percent of uncovered stent struts of about 1% at 18 months. In some embodiments, a lot of devices which includes the device has an average percent of uncovered stent struts of less than 11% at 4 months, less than 5% at 6 months, less than 5% at 8 months, and/or less than 1% at 18 months. In some embodiments, a lot of devices which includes the device has an average percent of uncovered stent struts of less than 11% at 4 months. In some embodiments, a lot of devices which includes the device has an average percent of uncovered stent struts of less than 5% at 6 months. In some embodiments, a lot of devices which includes the device has an average percent of uncovered stent struts of less than 5% at 8 months. In some embodiments, a lot of devices which includes the device has an average percent of uncovered stent struts of less than 1% at 18 months.

In some embodiments is a device comprising a stent and a coating on the stent wherein the coating comprises PLGA and crystalline sirolimus and wherein following implantation into a subject at least 0.02 mm of neointimal thickness exists on the stent on average at 4 months, at least 0.02 mm of neointimal thickness exists on the stent on average at 6 months, at least 0.06 mm of neointimal thickness exists on the stent on average at 8 months. In some embodiments is a device comprising a stent and a coating on the stent wherein the coating comprises PLGA and crystalline sirolimus and wherein following implantation into a subject at least 0.02 mm of neointimal thickness exists on the stent on average at 4 months. In some embodiments is a device comprising a stent and a coating on the stent wherein the coating comprises PLGA and crystalline sirolimus and wherein following implantation into a subject at least 0.05 mm of neointimal thickness exists on the stent on average at 4 months. In some embodiments is a device comprising a stent and a coating on the stent wherein the coating comprises PLGA and crystalline sirolimus and wherein following implantation into a subject at least 0.02 mm of neointimal thickness exists on the stent on average at 6 months. In some embodiments is a device comprising a stent and a coating on the stent wherein the coating comprises PLGA and crystalline sirolimus and wherein following implantation into a subject at least 0.075 mm of neointimal thickness exists on the stent on average at 6 months. In some embodiments is a device comprising a stent and a coating on the stent wherein the coating comprises PLGA and crystalline sirolimus and wherein following implantation into a subject at least 0.06 mm of neointimal thickness exists on the stent on average at 8 months. In some embodiments is a device comprising a stent and a coating on the stent wherein the coating comprises PLGA and crystalline sirolimus and wherein following implantation into a subject at least 0.1 mm of neointimal thickness exists on the stent on average at 8 months.

In some embodiments, at most 10% of the stent struts are covered by fibrin at 4 months following implantation, at most 4% of the stent struts are covered by fibrin at 6 months following implantation, or at most 1% of the stent struts are covered by fibrin at 8 months following implantation. In some embodiments, at most 10% of the stent struts are covered by fibrin at 4 months following implantation. In some embodiments, at most 4% of the stent struts are covered by fibrin at 6 months following implantation. In some embodiments, at most 1% of the stent struts are covered by fibrin at 8 months following implantation. In some embodiments, a MACE rate of a lot of devices which includes the device is at most 5%, between 1% and 5%, less than 5%, less than 4%, between 3% and 5%, or less than 3.5%. In some embodiments, a MACE rate of a lot of devices which includes the device is at most 5%. In some embodiments, a MACE rate of a lot of devices which includes the device is between 1% and 5%. In some embodiments, a MACE rate of a lot of devices which includes the device is less than 5%. In some embodiments, a MACE rate of a lot of devices which includes the device is less than 3.5%.

In some embodiments is a device comprising a stent and a coating on the stent wherein the coating comprises PLGA and crystalline sirolimus and wherein following implantation into a subject the late lumen loss is less than 0.55 mm, less than 0.50 mm, less than 0.45 mm, less than 0.40 mm, less than 0.35 mm, or less than 0.30 mm at 9 months following implantation. In some embodiments is a device comprising a stent and a coating on the stent wherein the coating comprises PLGA and crystalline sirolimus and wherein following implantation into a subject the late lumen loss is less than 0.55 mm at 9 months following implantation. In some embodiments is a device comprising a stent and a coating on the stent wherein the coating comprises PLGA and crystalline sirolimus and wherein following implantation into a subject the late lumen loss is less than 0.50 mm at 9 months following implantation. In some embodiments is a device comprising a stent and a coating on the stent wherein the coating comprises PLGA and crystalline sirolimus and wherein following implantation into a subject the late lumen loss is less than 0.45 mm at 9 months following implantation. In some embodiments is a device comprising a stent and a coating on the stent wherein the coating comprises PLGA and crystalline sirolimus and wherein following implantation into a subject the late lumen loss is less than 0.40 mm at 9 months following implantation. In some embodiments is a device comprising a stent and a coating on the stent wherein the coating comprises PLGA and crystalline sirolimus and wherein following implantation into a subject the late lumen loss is less than 0.35 mm at 9 months following implantation. In some embodiments is a device comprising a stent and a coating on the stent wherein the coating comprises PLGA and crystalline sirolimus and wherein following implantation into a subject the late lumen loss is less than 0.30 mm at 9 months following implantation. In some embodiments is a device comprising a stent and a coating on the stent wherein the coating comprises PLGA and crystalline sirolimus and wherein following implantation into a subject the late lumen loss is less than 0.25 mm at 9 months following implantation. In some embodiments is a device comprising a stent and a coating on the stent wherein the coating comprises PLGA and crystalline sirolimus and wherein following implantation into a subject the late lumen loss is less than 0.20 mm at 9 months following implantation.

In some embodiments is a device comprising a stent and a coating on the stent wherein the coating comprises PLGA and crystalline sirolimus and wherein following implantation into a subject the target lesion revascularization (TLR) is less than 1.5%, or less than 1.0% at 9 months following implantation. In some embodiments is a device comprising a stent and a coating on the stent wherein the coating comprises PLGA and crystalline sirolimus and wherein following implantation into a subject the target lesion revascularization (TLR) is less than 1.5% at 9 months following implantation. In some embodiments is a device comprising a stent and a coating on the stent wherein the coating comprises PLGA and crystalline sirolimus and wherein following implantation into a subject the target lesion revascularization (TLR) is less than 1.0% at 9 months following implantation.

In some embodiments is a device comprising a stent and a coating on the stent wherein the coating comprises PLGA and crystalline sirolimus and wherein following implantation into a subject the MACE rate is less than 6.5%, less than 6.0%, less than 5.5%, less than 5.0%, less than 4.5%, or less than 4.0% at 9 months following implantation. In some embodiments is a device comprising a stent and a coating on the stent wherein the coating comprises PLGA and crystalline sirolimus and wherein following implantation into a subject the MACE rate is less than 6.5% at 9 months following implantation. In some embodiments is a device comprising a stent and a coating on the stent wherein the coating comprises PLGA and crystalline sirolimus and wherein following implantation into a subject the MACE rate is less than 6.0% at 9 months following implantation. In some embodiments is a device comprising a stent and a coating on the stent wherein the coating comprises PLGA and crystalline sirolimus and wherein following implantation into a subject the MACE rate is less than 5.5% at 9 months following implantation. In some embodiments is a device comprising a stent and a coating on the stent wherein the coating comprises PLGA and crystalline sirolimus and wherein following implantation into a subject the MACE rate is less than 5.0% at 9 months following implantation. In some embodiments is a device comprising a stent and a coating on the stent wherein the coating comprises PLGA and crystalline sirolimus and wherein following implantation into a subject the MACE rate is less than 4.5% at 9 months following implantation. In some embodiments is a device comprising a stent and a coating on the stent wherein the coating comprises PLGA and crystalline sirolimus and wherein following implantation into a subject the MACE rate is less than 4.0% at 9 months following implantation. In some embodiments is a device comprising a stent and a coating on the stent wherein the coating comprises PLGA and crystalline sirolimus and wherein following implantation into a subject the MACE rate is less than 3.5% at 9 months following implantation. In some embodiments is a device comprising a stent and a coating on the stent wherein the coating comprises PLGA and crystalline sirolimus and wherein following implantation into a subject the MACE rate is less than 2.5% at 9 months following implantation.

Provided herein is a device comprising a stent; and a plurality of layers that form a laminate coating on said stent; wherein at least one of said layers comprises a bioabsorbable polymer and at least one of said layers comprises one or more active agents; wherein at least a portion of the active agent is in crystalline form.

Provided herein is a device comprising a stent; and a plurality of layers that form a laminate coating on said stent; wherein at least one of said layers comprises a bioabsorbable polymer and at least one of said layers comprises a pharmaceutical agent selected from rapamycin, a prodrug, a derivative, an analog, a hydrate, an ester, and a salt thereof; wherein at least a portion of the pharmaceutical agent is in crystalline form.

In some embodiments, the device has at least one pharmaceutical agent layer defined by a three-dimensional physical space occupied by crystal particles of said pharmaceutical agent and said three dimensional physical space is free of polymer. In some embodiments, at least some of the crystal particles in said three dimensional physical space defining said at least one pharmaceutical agent layer are in contact with polymer particles present in a polymer layer adjacent to said at least one pharmaceutical agent layer defined by said three-dimensional space free of polymer.

In some embodiments, the plurality of layers comprises a first polymer layer comprising a first bioabsorbable polymer and a second polymer layer comprising a second bioabsorbable polymer, wherein said at least one layer comprising said pharmaceutical agent is between said first polymer layer and said second polymer layer. In some embodiments, first and second bioabsorbable polymers are the same polymer. In some embodiments, the first and second bioabsorbable polymers are different. In some embodiments, the second polymer layer has at least one contact point with at least one particle of said pharmaceutical agent in said pharmaceutical agent layer and said second polymer layer has at least one contact point with said first polymer layer.

In some embodiments, the stent has a stent longitudinal axis; and said second polymer layer has a second polymer layer portion along said stent longitudinal wherein said second layer portion is free of contact with particles of said pharmaceutical agent. In some embodiments, the device has at least one pharmaceutical agent layer defined by a three-dimensional physical space occupied by crystal particles of said pharmaceutical agent and said three dimensional physical space is free of polymer.

The second polymer layer may have a layer portion defined along a longitudinal axis of the stent, said polymer layer portion having a thickness less than said maximum thickness of said second polymer layer; wherein said portion is free of contact with particles of said pharmaceutical agent.

The polymer layer portion may be a sub layer which, at least in part, extends along the abluminal surface of the stent along the longitudinal axis of the stent (where the longitudinal axis of the stent is the central axis of the stent along its tubular length). For example, when a coating is removed from the abluminal surface of the stent, such as when the stent is cut along its length, flattened, and the coating is removed by scraping the coating off using a scalpel, knife or other sharp tool, the coating that is removed (despite having a pattern consistent with the stent pattern) has a layer that can be shown to have the characteristics described herein. This may be shown by sampling multiple locations of the coating that is representative of the entire coating.

Alternatively, and/or additionally, since stents are generally comprised of a series of struts and voids. The methods provided herein advantageously allow for coatings extending around each strut, the layers of coating are likewise disposed around each strut. Thus, a polymer layer portion may be a layer which, at least, extends around each strut a distance from said strut (although the distance may vary where the coating thickness on the abluminal surface is different than the coating thickness on the luminal and/or sidewalls).

In some embodiments, the stent comprises at least one strut having a strut length along said stent longitudinal axis, wherein said second layer portion extends substantially along said strut length. In some embodiments, the stent has a stent length along said stent longitudinal axis and said second layer portion extends substantially along said stent length.

In some embodiments, the stent comprises at least five struts, each strut having a strut length along said stent longitudinal axis, wherein said second layer portion extends substantially along substantially the strut length of at least two struts. In some embodiments, the stent comprises at least five struts, each strut having a strut length along said stent longitudinal axis, wherein said second layer portion extends substantially along substantially the strut length of at least three struts. In some embodiments, the stent comprises at least five struts, each strut having a strut length along said stent longitudinal axis, wherein said second layer portion extends substantially along substantially the strut length of least four struts. In some embodiments, the stent comprises at least five struts, each strut having a strut length along said stent longitudinal axis, wherein said second layer portion extends substantially along substantially the strut length of all said at least five struts. In some embodiments, the stent has a stent length along said stent longitudinal axis and said second layer portion extends substantially along said stent length.

In some embodiments, the stent has a stent length along said stent longitudinal axis and said second layer portion extends along at least 50% of said stent length. In some embodiments, the stent has a stent length along said stent longitudinal axis and said second layer portion extends along at least 75% of said stent length. In some embodiments, the stent has a stent length along said stent longitudinal axis and said second layer portion extends along at least 85% of said stent length. In some embodiments, the stent has a stent length along said stent longitudinal axis and said second layer portion extends along at least 90% of said stent length. In some embodiments, the stent has a stent length along said stent longitudinal axis and said second layer portion extends along at least 99% of said stent length.

In some embodiments, the laminate coating has a total thickness and said second polymer layer portion has a thickness of from about 0.01% to about 10% of the total thickness of said laminate coating. In some embodiments, the laminate coating has a total thickness and said horizontal second polymer layer portion has a thickness of from about 1% to about 5% of the total thickness of said laminate coating. In some embodiments, the laminate coating has a total thickness of from about 5 μm to about 50 μm and said horizontal second polymer layer portion has a thickness of from about 0.001 μm to about 5 μm. In some embodiments, the laminate coating has a total thickness of from about 10 μm to about 20 μm and said second polymer layer portion has a thickness of from about 0.01 μm to about 5 μm. As used herein, the term “about” when referring to a laminate coating thickness means variations of any of 0.5%, 1%, 2%, 5%, 10%, 15%, 20%, 25%, 30%, and 50%, depending on the embodiment. For non-limiting example, a laminate coating thickness of 20 μm having a variation of 10% ranges from 18 μm to 22 μm, which is a range of 10% on either side of the target 20 μm. For non-limiting example, a layer portion having a thickness that is 1% of the total thickness of the laminate coating and having a variation of 0.5% means the layer portion may be from 0.5% to 1.5% of the total thickness of the laminate coating thickness. The coating can be conformal around the struts, isolated on the abluminal side, patterned, or otherwise optimized for the target tissue.

In some embodiments, the laminate coating is at least 25% by volume pharmaceutical agent. In some embodiments, the laminate coating is at least 35% by volume pharmaceutical agent. In some embodiments, the laminate coating is about 50% by volume pharmaceutical agent.

In some embodiments, at least a portion of the pharmaceutical agent is present in a phase separate from one or more phases formed by said polymer.

In some embodiments, the pharmaceutical agent is at least 50% crystalline. In some embodiments, the pharmaceutical agent is at least 75% crystalline. In some embodiments, the pharmaceutical agent is at least 90% crystalline. In some embodiments, the pharmaceutical agent is at least 95% crystalline. In some embodiments, the pharmaceutical agent is at least 99% crystalline.

In some embodiments, the stent has a stent longitudinal length and the coating has a coating outer surface along said stent longitudinal length, wherein said coating comprises pharmaceutical agent in crystalline form present in the coating below said coating outer surface. In some embodiments, the stent has a stent longitudinal length and the coating has a coating outer surface along said stent longitudinal length, wherein said coating comprises pharmaceutical agent in crystalline form present in the coating up to at least 1 μm below said coating outer surface. In some embodiments, the stent has a stent longitudinal length and the coating has a coating outer surface along said stent longitudinal length, wherein said coating comprises pharmaceutical agent in crystalline form present in the coating up to at least 5 μm below said coating outer surface.

In some embodiments, the coating exhibits an X-ray spectrum showing the presence of said pharmaceutical agent in crystalline form. In some embodiments, the coating exhibits a Raman spectrum showing the presence of said pharmaceutical agent in crystalline form. In some embodiments, the coating exhibits a Differential Scanning Calorimetry (DSC) curve showing the presence of said pharmaceutical agent in crystalline form. In some embodiments, said coating exhibits Wide Angle X-ray Scattering (WAXS) spectrum showing the presence of said pharmaceutical agent in crystalline form. In some embodiments, the coating exhibits a wide angle radiation scattering spectrum showing the presence of said pharmaceutical agent in crystalline form. In some embodiments, the coating exhibits an Infra Red (IR) spectrum showing the presence of said pharmaceutical agent in crystalline form.

In some embodiments, the stent has a stent longitudinal axis and a stent length along said stent longitudinal axis, wherein said coating is conformal to the stent along substantially said stent length.

In some embodiments, the stent has a stent longitudinal axis and a stent length along said stent longitudinal axis, wherein said coating is conformal to the stent along at least 75% of said stent length. In some embodiments, the stent has a stent longitudinal axis and a stent length along said stent longitudinal axis, wherein said coating is conformal to the stent along at least 85% of said stent length. In some embodiments, the stent has a stent longitudinal axis and a stent length along said stent longitudinal axis, wherein said coating is conformal to the stent along at least 90% of said stent length. In some embodiments, the stent has a stent longitudinal axis and a stent length along said stent longitudinal axis, wherein said coating is conformal to the stent along at least 95% of said stent length. In some embodiments, the stent has a stent longitudinal axis and a stent length along said stent longitudinal axis, wherein said coating is conformal to the stent along at least 99% of said stent length.

In some embodiments, the stent has a stent longitudinal axis and a plurality of struts along said stent longitudinal axis, wherein said coating is conformal to at least 50% of said struts. In some embodiments, the stent has a stent longitudinal axis and a plurality of struts along said stent longitudinal axis, wherein said coating is conformal to at least 75% of said struts. In some embodiments, the stent has a stent longitudinal axis and a plurality of struts along said stent longitudinal axis, wherein said coating is conformal to at least 90% of said struts. In some embodiments, the stent has a stent longitudinal axis and a plurality of struts along said stent longitudinal axis, wherein said coating is conformal to at least 99% of said struts. In some embodiments, the stent has a stent longitudinal axis and a stent length along said stent longitudinal axis, wherein an electron microscopy examination of the device shows said coating is conformal to said stent along at least 90% of said stent length.

In some embodiments, the stent has a stent longitudinal axis and a stent length along said stent longitudinal axis, wherein said coating has a substantially uniform thickness along substantially said stent length.

In some embodiments, the stent has a stent longitudinal axis and a stent length along said stent longitudinal axis, wherein said coating has a substantially uniform thickness along at least 75% of said stent length. In some embodiments, the stent has a stent longitudinal axis and a stent length along said stent longitudinal axis, wherein said coating has a substantially uniform thickness along at least 95% of said stent length.

In some embodiments, the stent has a stent longitudinal axis and a stent length along said stent longitudinal axis, wherein said coating has an average thickness determined by an average calculated from coating thickness values measured at a plurality of points along said stent longitudinal axis; wherein a thickness of the coating measured at any point along stent longitudinal axis is from about 75% to about 125% of said average thickness. In some embodiments, the stent has a stent longitudinal axis and a stent length along said stent longitudinal axis, wherein said coating has an average thickness determined by an average calculated from coating thickness values measured at a plurality of points along said stent longitudinal axis; wherein a thickness of the coating measured at any point along stent longitudinal axis is from about 95% to about 105% of said average thickness. As used herein, the term “about” when referring to a coating thickness means variations of any of 0.5%, 1%, 2%, 5%, 10%, 15%, 20%, 25%, 30%, and 50%, depending on the embodiment. For non-limiting example, a coating thickness at a point along the stent longitudinal axis which is 75% of the average thickness and having a variation of 10% may actually be anywhere from 65% to 85% of the average thickness.

Provided herein is a device comprising: a stent; and a plurality of layers that form a laminate coating on said stent, wherein a first layer comprises a first bioabsorbable polymer, a second layer comprises a pharmaceutical agent, a third layer comprises a second bioabsorbable polymer, a fourth layer comprises the pharmaceutical agent, and a fifth layer comprises a third bioabsorbable polymer, wherein the pharmaceutical agent is selected from rapamycin, a prodrug, a derivative, an analog, a hydrate, an ester, and a salt thereof, and wherein at least a portion of the pharmaceutical agent is in crystalline form.

In some embodiments, at least two of said first bioabsorbable polymer, said second bioabsorbable polymer and said third bioabsorbable polymer are the same polymer. In some embodiments, the first bioabsorbable polymer, the second bioabsorbable polymer and the third bioabsorbable polymer are the same polymer. In some embodiments, at least two of said first bioabsorbable polymer, said second bioabsorbable polymer and said third bioabsorbable polymer are different polymers. In some embodiments, the first bioabsorbable polymer, said second bioabsorbable polymer and said third bioabsorbable polymer are different polymers.

In some embodiments, the third layer has at least one contact point with particles of said pharmaceutical agent in said second layer; and said third layer has at least one contact point with said first layer.

In some embodiments, at least two of the first polymer, the second polymer, and the third polymer are the same polymer, and wherein said same polymer comprises a PLGA copolymer. In some embodiments, the third polymer has an in vitro dissolution rate higher than the in vitro dissolution rate of the first polymer. In some embodiments, the third polymer is PLGA copolymer with a ratio of about 40:60 to about 60:40 and the first polymer is a PLGA copolymer with a ratio of about 70:30 to about 90:10. In some embodiments, the third polymer is PLGA copolymer having a molecular weight of about 10 kD (weight average molecular weight) and the second polymer is a PLGA copolymer having a molecular weight of about 19 kD (weight average molecular weight). In some embodiments, the first polymer, the second polymer, and the third polymer each comprise a PLGA copolymer having a number average molecular weight of between about 9.5 kD and about 25 kD. In some embodiments, the first polymer, the second polymer, and the third polymer each comprise a PLGA copolymer having a number average molecular weight of between about 14.5 kD and about 15 kD. As used herein, the term “about,” when referring to a copolymer ratio, means variations of any of 0.5%, 1%, 2%, 5%, 10%, 15%, 20%, 25%, 30%, and 50%, depending on the embodiment. For example, a copolymer ratio of 40:60 having a variation of 10% ranges from 35:65 to 45:55, which is a range of 10% of the total (100) about the target. As used herein, the term “about” when referring to a polymer molecular weight means variations of any of 0.5%, 1%, 2%, 5%, 10%, 15%, 20%, 25%, 30%, and 50%, depending on the embodiment. For example, a polymer molecular weight of 10 kD (weight average molecular weight) having a variation of 10% ranges from 9 kD to 11 kD, which is a range of 10% of the target 10 kD on either side of the target 10 kD.

In some embodiments, measuring the in vitro dissolution rate of said polymers comprises contacting the device with elution media and determining polymer weight loss at one or more selected time points. In some embodiments, measuring the in vitro dissolution rate of said polymers comprises contacting the device with elution media and determining polymer weight loss at one or more selected time points.

Provided herein is a device, comprising: a stent; and a coating on said stent comprising a first bioabsorbable polymer, a second bioabsorbable polymer; and pharmaceutical agent selected from rapamycin, a prodrug, a derivative, an analog, a hydrate, an ester, and a salt thereof wherein at least a portion of the pharmaceutical agent is in crystalline form, and wherein the first polymer has an in vitro dissolution rate higher than the in vitro dissolution rate of the second polymer.

In some embodiments, the first polymer is PLGA copolymer with a ratio of about 40:60 to about 60:40 and the second polymer is a PLGA copolymer with a ratio of about 70:30 to about 90:10. In some embodiments, the first polymer is PLGA copolymer having a molecular weight of about 10 kD (weight average molecular weight) and the second polymer is a PLGA copolymer having a molecular weight of about 19 kD (weight average molecular weight). In some embodiments, the coating comprises a PLGA copolymer having a number average molecular weight of between about 9.5 kD and about 25 kD. In some embodiments, the coating comprises a PLGA copolymer having a number average molecular weight of between about 14.5 kD and about 15 kD. In some embodiments, measuring the in vitro dissolution rate of said polymers comprises contacting the device with elution media and determining polymer weight loss at one or more selected time points. As used herein, the term “about,” when referring to a copolymer ratio, means variations of any of 0.5%, 1%, 2%, 5%, 10%, 15%, 20%, 25%, 30%, and 50%, depending on the embodiment. For example, a copolymer ratio of 40:60 having a variation of 10% ranges from 35:65 to 45:55, which is a range of 10% of the total (100) about the target. As used herein, the term “about” when referring to a polymer molecular weight means variations of any of 0.5%, 1%, 2%, 5%, 10%, 15%, 20%, 25%, 30%, and 50%, depending on the embodiment. For example, a polymer molecular weight of 10 kD (weight average molecular weight) having a variation of 10% ranges from 9 kD to 11 kD, which is a range of 10% of the target 10 kD on either side of the target 10 kD.

Provided herein is a device comprising a stent; and a plurality of layers that form a laminate coating on said stent; wherein at least one of said layers comprises a first bioabsorbable polymer, at least one of said layers comprises a second bioabsorbable polymer, and at least one of said layers comprises one or more active agents; wherein at least a portion of the active agent is in crystalline form, and wherein the first polymer has an in vitro dissolution rate higher than the in vitro dissolution rate of the second polymer.

Provided herein is a device comprising a stent; and a plurality of layers that form a laminate coating on said stent; wherein at least one of said layers comprises a first bioabsorbable polymer, at least one of said layers comprises a second bioabsorbable polymer, and at least one of said layers comprises a pharmaceutical agent selected from rapamycin, a prodrug, a derivative, an analog, a hydrate, an ester, and a salt thereof; wherein at least a portion of the pharmaceutical agent is in crystalline form and wherein the first polymer has an in vitro dissolution rate higher than the in vitro dissolution rate of the second polymer.

In some embodiments, the first polymer is PLGA copolymer with a ratio of about 40:60 to about 60:40 and the second polymer is a PLGA copolymer with a ratio of about 70:30 to about 90:10. In some embodiments, the first polymer is PLGA copolymer having a molecular weight of about 10 kD (weight average molecular weight) and the second polymer is a PLGA copolymer having a molecular weight of about 19 kD (weight average molecular weight). In some embodiments, at least one of the first coating and the second coating comprises a PLGA copolymer having a number average molecular weight of between about 9.5 kD and about 25 kD. In some embodiments, at least one of the first coating and the second coating comprises a PLGA copolymer having a number average molecular weight of between about 14.5 kD and about 15 kD. In some embodiments, measuring the in vitro dissolution rate comprises contacting the device with elution media and determining polymer weight loss at one or more selected time points. As used herein, the term “about,” when referring to a copolymer ratio, means variations of any of 0.5%, 1%, 2%, 5%, 10%, 15%, 20%, 25%, 30%, and 50%, depending on the embodiment. For example, a copolymer ratio of 40:60 having a variation of 10% ranges from 35:65 to 45:55, which is a range of 10% of the total (100) about the target. As used herein, the term “about” when referring to a polymer molecular weight means variations of any of 0.5%, 1%, 2%, 5%, 10%, 15%, 20%, 25%, 30%, and 50%, depending on the embodiment. For example, a polymer molecular weight of 10 kD (weight average molecular weight) having a variation of 10% ranges from 9 kD to 11 kD, which is a range of 10% of the target 10 kD on either side of the target 10 kD.

Provided herein is a device comprising a stent; and a plurality of layers that form a laminate coating on said stent; wherein at least one of said layers comprises a bioabsorbable polymer, at least one of said layers comprises a first active agent and at least one of said layers comprises a second active agent; wherein at least a portion of first and/or second active agents is in crystalline form.

In some embodiments, the bioabsorbable polymer is selected from the group PLGA, PGA poly(glycolide), LPLA poly(l-lactide), DLPLA poly(dl-lactide), PCL poly(e-caprolactone) PDO, poly(dioxolane) PGA-TMC, 85/15 DLPLG p(dl-lactide-co-glycolide), 75/25 DLPLG, 65/35 DLPLG, 50/50 DLPLG, TMC poly(trimethylcarbonate), poly(anhydrides) such as p(CPP:SA) poly(1,3-bis-p-(carboxyphenoxy)propane-co-sebacic acid). In some embodiments, the polymer comprises an intimate mixture of two or more polymers.

In some embodiments, the first and second active agents are independently selected from pharmaceutical agents and active biological agents.

In some embodiments, the stent is formed of stainless steel material. In some embodiments, the stent is formed of a material comprising a cobalt chromium alloy. In some embodiments, the stent is formed from a material comprising the following percentages by weight: about 0.05 to about 0.15 C, about 1.00 to about 2.00 Mn, about 0.04 Si, about 0.03 P, about 0.3 S, about 19.0 to about 21.0 Cr, about 9.0 to about 11.0 Ni, about 14.0 to about 16.00 W, about 3.0 Fe, and Bal. Co. In some embodiments, the stent is formed from a material comprising at most the following percentages by weight: about 0.025 C, about 0.15 Mn, about 0.15 Si, about 0.015 P, about 0.01 S, about 19.0 to about 21.0 Cr, about 33 to about 37 Ni, about 9.0 to about 10.5 Mo, about 1.0 Fe, about 1.0 Ti, and Bal. Co. In some embodiments, the stent is formed from a material comprising L605 alloy. In some embodiments, the stent is formed from a material comprising MP35N alloy. In some embodiments, the stent is formed from a material comprising the following percentages by weight: about 35 Ni, about 35 Cr, about 20 Co, and about 10 Mo. In some embodiments, the stent is formed from a material comprising a cobalt chromium nickel alloy. In some embodiments, the stent is formed from a material comprising Elgiloy®/Phynox®. In some embodiments, the stent is formed from a material comprising the following percentages by weight: about 39 to about 41 Co, about 19 to about 21 Cr, about 14 to about 16 Ni, about 6 to about 8 Mo, and Balance Fe. In some embodiments, the stent is formed of a material comprising a platinum chromium alloy. In some embodiments, the stent is formed of an alloy as described in U.S. Pat. No. 7,329,383 incorporated in its entirety herein by reference. In some embodiments, the stent is formed of an alloy as described in U.S. patent application Ser. No. 11/780,060 incorporated in its entirety herein by reference. In some embodiments, the stent may be formed of a material comprising stainless steel, 316L stainless steel, BioDur® 108 (UNS S29108), 304L stainless steel, and an alloy including stainless steel and 5-60% by weight of one or more radiopaque elements such as Pt, IR, Au, W, PERSS® as described in U.S. Publication No. 2003/001830 incorporated in its entirety herein by reference, U.S. Publication No. 2002/0144757 incorporated in its entirety herein by reference, and U.S. Publication No. 2003/0077200 incorporated in its entirety herein by reference, nitinol, a nickel-titanium alloy, cobalt alloys, Elgiloy®, L605 alloys, MP35N alloys, titanium, titanium alloys, Ti-6Al-4V, Ti-50Ta, Ti-10Ir, platinum, platinum alloys, niobium, niobium alloys, Nb-1Zr, Co-28Cr-6Mo, tantalum, and tantalum alloys. Other examples of materials are described in U.S. Publication No. 2005/0070990 incorporated in its entirety herein by reference, and U.S. Publication No. 2006/0153729 incorporated in its entirety herein by reference. Other materials include elastic biocompatible metal such as superelastic or pseudo-elastic metal alloys, as described, for example in Schetsky, L. McDonald, “Shape Memory Alloys”, Encyclopedia of Chemical Technology (3d Ed), John Wiley & Sons 1982, vol. 20 pp. 726-736 incorporated herein by reference, and U.S. Publication No. 2004/0143317 incorporated in its entirety herein by reference. As used herein, the term “about,” when referring to a weight percentage of stent material, means variations of any of 0.5%, 1%, 2%, 5%, 10%, 15%, 20%, 25%, 30%, and 50% of the total weight percent (i.e. 100%) on either side (+/−) of the weight percentage, depending on the embodiment. For example, a weight percentage of stent material of 3.0 Fe having a variation of 1% ranges from 2.0 to 4.0, which is a range of 1% of the total (100) on either side of the target 3.0.

In some embodiments, the stent has a thickness of from about 50% to about 90% of a total thickness of said device. In some embodiments, the device has a thickness of from about 20 μm to about 500 μm. In some embodiments, the device has a thickness of about 90 μm or less. In some embodiments, the laminate coating has a thickness of from about 5 μm to about 50 μm. In some embodiments, the laminate coating has a thickness of from about 10 μm to about 20 μm. In some embodiments, the stent has a thickness of from about 50 μm to about 80 μm. As used herein, the term “about” when referring to a device thickness or coating thickness or laminate coating thickness means variations of any of 0.5%, 1%, 2%, 5%, 10%, 15%, 20%, 25%, 30%, and 50%, depending on the embodiment. For non-limiting example, a device thickness of 20 μm having a variation of 10% ranges from 18 μm to 22 μm, which is a range of 10% on either side of the target 20 μm. The coating can be conformal around the struts, isolated on the abluminal side, patterned, or otherwise optimized for the particular target tissue.

Provided herein is a device comprising: a stent, wherein the stent is formed from a material comprising the following percentages by weight: 0.05-0.15 C, 1.00-2.00 Mn, 0.040 Si, 0.030 P, 0.3 S, 19.00-21.00 Cr, 9.00-11.00 Ni, 14.00-16.00 W, 3.00 Fe, and Bal. Co; and a plurality of layers that form a laminate coating on said stent, wherein a first layer comprises a first bioabsorbable polymer, a second layer comprises a pharmaceutical agent, a third layer comprises a second bioabsorbable polymer, a fourth layer comprises the pharmaceutical agent, and a fifth layer comprises a third bioabsorbable polymer, wherein the pharmaceutical agent is selected from rapamycin, a prodrug, a derivative, an analog, a hydrate, an ester, and a salt thereof, wherein at least a portion of the pharmaceutical agent is in crystalline form, and wherein at least one of said first polymer, second polymer and third polymer comprises a PLGA copolymer.

In some embodiments, the device has a pharmaceutical agent content of from about 0.5 μg/mm to about 20 μg/mm. In some embodiments, the device has a pharmaceutical agent content of from about 8 μg/mm to about 12 μg/mm. In some embodiments, the device has a pharmaceutical agent content of from about 5 μg to about 500 μg. In some embodiments, the device has a pharmaceutical agent content of from about 100 μg to about 160 μg. As used herein, the term “about” when referring to an active agent content (or pharmaceutical agent content) means variations of any of 0.5%, 1%, 2%, 5%, 10%, 15%, 20%, 25%, 30%, and 50%, depending on the embodiment. For non-limiting example, an active agent content (or pharmaceutical agent content) of 120 μg having a variation of 10% ranges from 108 μg to 132 μg, which is a range of 10% on either side of the target 120 μg. Where content is expressed herein in units of μg/mm, however, this may simply be converted to μg/mm2 or another amount per area (e.g., μg/cm2), or vice versa. Similarly, where content is expressed in terms of μg, this may be simply converted to a per-area or per-length term, or vice versa as needed.

Provided herein is a method of preparing a device comprising a stent and a plurality of layers that form a laminate coating on said stent; said method comprising: (a) providing a stent; (b) forming a plurality of layers on said stent to form said laminate coating on said stent; wherein at least one of said layers comprises a bioabsorbable polymer and at least one of said layers comprises one or more active agents; wherein at least a portion of the active agent is in crystalline form.

Provided herein is a method of preparing a device comprising a stent and a plurality of layers that form a laminate coating on said stent; said method comprising: (a) providing a stent; (b) forming a plurality of layers to form said laminate coating on said stent; wherein at least one of said layers comprises a bioabsorbable polymer and at least one of said layers comprises a pharmaceutical agent selected from rapamycin, a prodrug, a derivative, an analog, a hydrate, an ester, and a salt thereof; wherein at least a portion of the pharmaceutical agent is in crystalline form.

Provided herein is a method of preparing a device comprising a stent and a plurality of layers that form a laminate coating on said stent; said method comprising: (a) providing a stent; (b) forming a plurality of layers to form said laminate coating on said stent; wherein at least one of said layers comprises a bioabsorbable polymer and at least one of said layers comprises a pharmaceutical agent selected from rapamycin, a prodrug, a derivative, an analog, a hydrate, an ester, and a salt thereof; wherein at least a portion of the pharmaceutical agent is in crystalline form, wherein said method comprises forming at least one pharmaceutical agent layer defined by a three-dimensional physical space occupied by crystal particles of said pharmaceutical agent and said three dimensional physical space is free of polymer.

Provided herein is a method of preparing a device comprising a stent and a plurality of layers that form a laminate coating on said stent; said method comprising: (a) providing a stent; (b) discharging at least one pharmaceutical agent and/or at least one active biological agent in dry powder form through a first orifice; (c) forming a supercritical or near supercritical fluid solution comprising at least one supercritical fluid solvent and at least one polymer and discharging said supercritical or near supercritical fluid solution through a second orifice under conditions sufficient to form solid particles of the polymer; (d) depositing the polymer and pharmaceutical agent and/or active biological agent particles onto said substrate, wherein an electrical potential is maintained between the substrate and the polymer and pharmaceutical agent and/or active biological agent particles, thereby forming said coating; and (e) sintering said polymer under conditions that do not substantially modify a morphology of said pharmaceutical agent and/or activity of said biological agent.

In some embodiments, step (b) comprises discharging a pharmaceutical agent selected from rapamycin, a prodrug, a derivative, an analog, a hydrate, an ester, and a salt thereof; wherein at least a portion of the pharmaceutical agent is in crystalline form. In some embodiments, step (c) comprises forming solid particles of a bioabsorbable polymer.

In some embodiments, step (e) comprises forming a polymer layer having a length along a horizontal axis of said device wherein said polymer layer has a layer portion along said length, wherein said layer portion is free of pharmaceutical agent.

In some embodiments, step (e) comprises contacting said polymer with a densified fluid. In some embodiments, step (e) comprises contacting said polymer with a densified fluid for a period of time at a temperature of from about 5° C. and 150° C. and a pressure of from about 10 psi to about 500 psi. In some embodiments, step (e) comprises contacting said polymer with a densified fluid for a period of time at a temperature of from about 25° C. and 95° C. and a pressure of from about 25 psi to about 100 psi. In some embodiments, step (e) comprises contacting said polymer with a densified fluid for a period of time at a temperature of from about 50° C. and 85° C. and a pressure of from about 35 psi to about 65 psi. The term “about” when used in reference to a temperature in the coating process means variations of any of 0.5%, 1%, 2%, 5%, 10%, 15%, 20%, 25%, 30%, and 50%, on either side of the target or on a single side of the target, depending on the embodiment. For non-limiting example, for a temperature of 150° C. having a variability of 10% on either side of the target (of 150° C.), the temperature would range from 135° C. to 165° C. The term “about” when used in reference to a pressure in the coating process means variations of any of 0.5%, 1%, 2%, 5%, 10%, 15%, 20%, 25%, 30%, and 50%, depending on the embodiment. For non-limiting example, for a pressure of 100 psi having a variability of 10% on either side of the target (of 100 psi), the pressure would range from 90 psi to 110 psi.

Provided herein is a method of preparing a device comprising a stent and a plurality of layers that form a laminate coating on said stent; said method comprising: (a) providing a stent; (b) forming a supercritical or near supercritical fluid solution comprising at least one supercritical fluid solvent and a first polymer, discharging said supercritical or near supercritical fluid solution under conditions sufficient to form solid particles of said first polymer, depositing said first polymer particles onto said stent, wherein an electrical potential is maintained between the stent and the first polymer, and sintering said first polymer; (c) depositing pharmaceutical agent particles in dry powder form onto said stent, wherein an electrical potential is maintained between the stent and said pharmaceutical agent particles; and (d) forming a supercritical or near supercritical fluid solution comprising at least one supercritical fluid solvent and a second polymer and discharging said supercritical or near supercritical fluid solution under conditions sufficient to form solid particles of said second polymer, wherein an electrical potential is maintained between the stent and the second polymer, and sintering said second polymer.

In some embodiments, step (c) and step (d) are repeated at least once. In some embodiments, steps (c) and step (d) are repeated 2 to 20 times.

In some embodiments, the pharmaceutical agent is selected from rapamycin, a prodrug, a derivative, an analog, a hydrate, an ester, and a salt thereof; wherein at least a portion of the pharmaceutical agent is in crystalline form. In some embodiments, the first and second polymers are bioabsorbable.

In some embodiments, step (d) comprises forming a polymer layer having a length along a horizontal axis of said device wherein said polymer layer has a layer portion along said length, wherein said layer portion is free of pharmaceutical agent.

In some embodiments, sintering said first and/or sintering said second polymer comprises contacting said first and/or second polymer with a densified fluid.

In some embodiments, the contacting step is carried out for a period of from about 1 minute to about 60 minutes. In some embodiments, the contacting step is carried out for a period of from about 10 minutes to about 30 minutes.

In some embodiments, maintaining said electrical potential between said polymer particles and or pharmaceutical agent particles and said stent comprises maintaining a voltage of from about 5 kvolts to about 100 kvolts. In some embodiments, maintaining said electrical potential between said polymer particles and or pharmaceutical agent particles and said stent comprises maintaining a voltage of from about 20 kvolts to about 30 kvolts.

Provided herein is a device prepared by a process comprising a method as described herein.

Provided herein is method of treating a subject comprising delivering a device as described herein in a body lumen of the subject.

Provided herein is a method of treating a subject comprising delivering in the body of the subject a device comprising: a stent, wherein the stent is formed from a material comprising the following percentages by weight: 0.05-0.15 C, 1.00-2.00 Mn, 0.040 Si, 0.030 P, 0.3 S, 19.00-21.00 Cr, 9.00-11.00 Ni, 14.00-16.00 W, 3.00 Fe, and Bal. Co; and a plurality of layers that form a laminate coating on said stent, wherein a first layer comprises a first bioabsorbable polymer, a second layer comprises a pharmaceutical agent, a third layer comprises a second bioabsorbable polymer, a fourth layer comprises the pharmaceutical agent, and a fifth layer comprises a third bioabsorbable polymer, wherein the pharmaceutical agent is selected from rapamycin, a prodrug, a derivative, an analog, a hydrate, an ester, and a salt thereof, wherein at least a portion of the pharmaceutical agent is in crystalline form, and wherein at least one of said first polymer, second polymer and third polymer comprises a PLGA copolymer.

In some embodiments, the device has a pharmaceutical agent content of from about 0.5 μg/mm to about 20 μg/mm. In some embodiments, the device has a pharmaceutical agent content of from about 8 μg/mm to about 12 μg/mm. In some embodiments, the device has a pharmaceutical agent content of from about 100 μg to about 160 μg. In some embodiments, the device has a pharmaceutical agent content of from about 120 μg to about 150 μg. As used herein, the term “about” when referring to a pharmaceutical agent content means variations of any of 0.5%, 1%, 2%, 5%, 10%, 15%, 20%, 25%, 30%, and 50%, depending on the embodiment. For non-limiting example, a pharmaceutical agent content of 120 μg having a variation of 10% ranges from 108 μg to 132 μg, which is a range of 10% on either side of the target 120 μg. Where content is expressed herein in units of μg/mm, however, this may simply be converted to μg/mm² or another amount per area (e.g., μg/cm²), or vice versa, or converted to a total pharmaceutical content by multiplying by the area or length as needed.

In some embodiments, the device has an initial pharmaceutical agent amount and the amount of pharmaceutical agent delivered by said device to vessel wall tissue of said subject is higher than the amount of pharmaceutical agent delivered by a conventional drug eluting stent having the same initial pharmaceutical agent content as the initial pharmaceutical agent content of said device. In some embodiments, the amount of pharmaceutical agent delivered by said device to vessel wall tissue of said subject is at least 25% more that the amount of pharmaceutical agent delivered to vessel wall tissue of said subject by said conventional drug eluting stent. In some embodiments, the method comprises treating restenosis in a blood vessel of said the subject. In some embodiments, the subject is selected from a pig, a rabbit and a human.

“Vessel wall tissue” as used herein includes the tissue surrounding the lumen of a vessel, including the endothelium, neointima, tunica media, IEL (internal elastic lamina), EEL (external elastic lamina), and the tunica adventitia.

Provided herein is a device comprising: a stent; and a plurality of layers on said stent; wherein at least one of said layers comprises a bioabsorbable polymer and at least one of said layers comprises a pharmaceutical agent selected from rapamycin, a prodrug, a derivative, an analog, a hydrate, an ester, and a salt thereof; wherein said device provides an in vitro pharmaceutical agent elution profile wherein said elution profile shows about 5% to about 25% of pharmaceutical agent is eluted one day after the device is contacted with elution media; 15% to about 45% of pharmaceutical agent is eluted 7 days after the device is contacted with elution media; about 25% to about 60% of pharmaceutical agent is eluted 14 days after the device is contacted with elution media; about 35% to about 70% of pharmaceutical agent is eluted 21 days after the device is contacted with elution media; and about 40% to about 100% of pharmaceutical agent is eluted 28 days after the device is contacted with elution media. As used herein, the term “about” when used in reference to percent elution means variations of any of 0.01%, 0.05%, 0.1%, 0.5%, 1%, 2%, 5%, 10%, 15%, 20%, 25%, 30%, and 50% on either side of the percent elution or on a single side of the aspect target, depending on the embodiment. For non-limiting example, for an elution of 25% having a variation of 5%, this could mean 25% plus or minus 5%—equating to a range of 20% to 30%.

Provided herein is a device comprising a stent; and a plurality of layers on said stent; wherein at least one of said layers comprises a bioabsorbable polymer and at least one of said layers comprises a pharmaceutical agent selected from rapamycin, a prodrug, a derivative, an analog, a hydrate, an ester, and a salt thereof; wherein said device provides an in vitro pharmaceutical agent elution profile wherein said elution profile shows about 7% to about 15% of pharmaceutical agent is eluted one day after the device is contacted with elution media; 25% to about 35% of pharmaceutical agent is eluted 7 days after the device is contacted with elution media; about 35% to about 55% of pharmaceutical agent is eluted 14 days after the device is contacted with elution media; about 45% to about 60% of pharmaceutical agent is eluted 21 days after the device is contacted with elution media; and about 50% to about 70% of pharmaceutical agent is eluted 28 days after the device is contacted with elution media. As used herein, the term “about” when used in reference to percent elution means variations of any of 0.01%, 0.05%, 0.1%, 0.5%, 1%, 2%, 5%, 10%, 15%, 20%, 25%, 30%, and 50% on either side of the percent elution or on a single side of the aspect target, depending on the embodiment. For non-limiting example, for an elution of 25% having a variation of 5%, this could mean 25% plus or minus 5%—equating to a range of 20% to 30%.

Provided herein is a device comprising a stent; and a plurality of layers on said stent; wherein at least one of said layers comprises a bioabsorbable polymer and at least one of said layers comprises a pharmaceutical agent selected from rapamycin, a prodrug, a derivative, an analog, a hydrate, an ester, and a salt thereof; wherein said device provides an in vitro pharmaceutical agent elution profile wherein said elution profile shows at least 5% of pharmaceutical agent is eluted one day after the device is contacted with elution media; at least 15% of pharmaceutical agent is eluted 7 days after the device is contacted with elution media; at least 25% of pharmaceutical agent is eluted 14 days after the device is contacted with elution media; at least 30% of pharmaceutical agent is eluted 21 days after the device is contacted with elution media; at least 40% of pharmaceutical agent is eluted 28 days after the device is contacted with elution media.

Provided herein is a device comprising a stent; and a plurality of layers on said stent; wherein at least one of said layers comprises a bioabsorbable polymer and at least one of said layers comprises a pharmaceutical agent selected from rapamycin, a prodrug, a derivative, an analog, a hydrate, an ester, and a salt thereof; wherein said device provides an in vitro pharmaceutical agent elution profile wherein said elution profile shows about 10% of pharmaceutical agent is eluted one day after the device is contacted with elution media; about 30% of pharmaceutical agent is eluted 7 days after the device is contacted with elution media; about 45% of pharmaceutical agent is eluted 14 days after the device is contacted with elution media; about 50% of pharmaceutical agent is eluted 21 days after the device is contacted with elution media; about 60% of pharmaceutical agent is eluted 28 days after the device is contacted with elution media.

Provided herein is a device comprising a stent; and a plurality of layers on said stent; wherein at least one of said layers comprises a bioabsorbable polymer and at least one of said layers comprises a pharmaceutical agent selected from rapamycin, a prodrug, a derivative, an analog, a hydrate, an ester, and a salt thereof; wherein said device provides an in vitro pharmaceutical agent elution profile wherein said elution profile shows about 10% to about 75% of pharmaceutical agent is eluted at week 1 after the device is contacted with elution media, about 25% to about 85% of pharmaceutical agent is eluted at week 2 and about 50% to about 100% of pharmaceutical agent is eluted at week 10. As used herein, the term “about” when used in reference to percent elution means variations of any of 0.01%, 0.05%, 0.1%, 0.5%, 1%, 2%, 5%, 10%, 15%, 20%, 25%, 30%, and 50% on either side of the percent elution or on a single side of the aspect target, depending on the embodiment. For non-limiting example, for an elution of 25% having a variation of 5%, this could mean 25% plus or minus 5%—equating to a range of 20% to 30%.

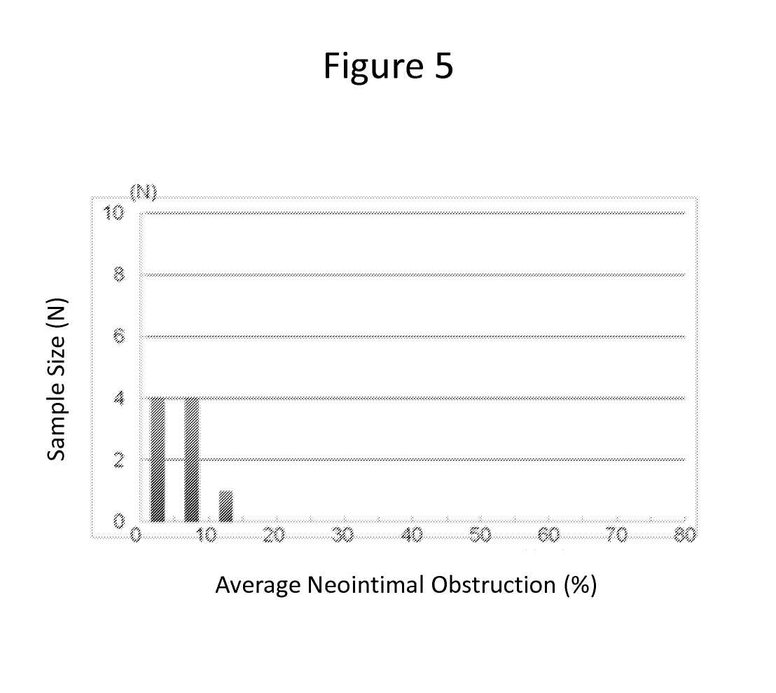

Provided herein is a device comprising: a stent; and a plurality of layers on said stent; wherein at least one of said layers comprises a bioabsorbable polymer and at least one of said layers comprises a pharmaceutical agent selected from rapamycin, a prodrug, a derivative, an analog, a hydrate, an ester, and a salt thereof; wherein said device provides an in vitro pharmaceutical agent elution profile shown in FIG. 5.

In some embodiments, the in vitro pharmaceutical agent elution profile is determined by a procedure comprising: (i) contacting the device with an elution media comprising 5% ethanol by volume wherein the pH of the media is about 7.4 and wherein the device is contacted with the elution media at a temperature of about 37° C.; (ii) optionally agitating the elution media during the contacting step in (i); (iii) removing the elution media at designated time points; and (iv) assaying the removed elution media to determine pharmaceutical agent content.

In some embodiments, the in vitro pharmaceutical agent elution profile is determined by a procedure comprising: (i) contacting the device with an elution media comprising 5% ethanol by volume, wherein the pH of the media is about 7.4 and wherein the device is contacted with the elution media at a temperature of about 37° C.; (ii) optionally agitating the elution media during the contacting step in (i); (iii) removing said device from the elution media at designated time points; and (iv) assaying the elution media to determine pharmaceutical agent content.

In some embodiments, the in vitro pharmaceutical agent elution profile is determined in the absence of agitation.

In some embodiments, the procedure further comprises: (v) determining polymer weight loss by comparing the weight of the device before and after the contacting step and adjusting for the amount of pharmaceutical agent eluted into the elution media as determined in step (iv). In some embodiments, step (v) shows at least 50% of polymer is released into the media after the device is contacted with the media for 90 days or more. In some embodiments, step (v) shows at least 75% of polymer is released into the media after the device is contacted with the media for 90 days or more.

In some embodiments, step (v) shows at least 85% of polymer is released into the media after the device is contacted with the media for 90 days or more. In some embodiments, step (v) shows at least 50% of polymer is released into the media after the device is contacted with the media for about 90 days. In some embodiments, step (v) shows at least 75% of polymer is released into the media after the device is contacted with the media for about 90 days. In some embodiments, step (v) shows at least 85% of polymer is released into the media after the device is contacted with the media for about 90 days. In some embodiments, step (v) shows at least 95% of polymer is released into the media after the device is contacted with the media for about 90 days. In some embodiments, step (v) shows up to 100% of polymer is released into the media after the device is contacted with the media for about 90 days. As used herein, the term “about” when referring to the media contact time can vary up to 1%, 5%, 10%, 20%, 25%, 6 hrs, 12 hrs, 24 hrs, 1 day, 2 days, 3 days, 5 days, or 7 days.

Provided herein is a device comprising: a stent; and a plurality of layers on said stent; wherein at least one of said layers comprises a bioabsorbable polymer and at least one of said layers comprises a pharmaceutical agent selected from rapamycin, a prodrug, a derivative, an analog, a hydrate, an ester, and a salt thereof; wherein said device provides an in vitro pharmaceutical agent elution profile wherein said elution profile shows about 1% to about 35% of pharmaceutical agent is eluted one hour after the device is contacted with elution media; 5% to about 45% of pharmaceutical agent is eluted 3 hours after the device is contacted with elution media; about 30% to about 70% of pharmaceutical agent is eluted 1 day after the device is contacted with elution media; about 40% to about 80% of pharmaceutical agent is eluted 3 days after the device is contacted with elution media; about 50% to about 90% of pharmaceutical agent is eluted 10 days after the device is contacted with elution media about 55% to about 95% of pharmaceutical agent is eluted 15 days after the device is contacted with elution media; and about 60% to about 100% of pharmaceutical agent is eluted 20 days after the device is contacted with elution media. As used herein, the term “about” when used in reference to percent elution means variations of any of 0.01%, 0.05%, 0.1%, 0.5%, 1%, 2%, 5%, 10%, 15%, 20%, 25%, 30%, and 50% on either side of the percent elution or on a single side of the aspect target, depending on the embodiment. For non-limiting example, for an elution of 25% having a variation of 5%, this could mean 25% plus or minus 5%—equating to a range of 20% to 30%.

Provided herein is a device comprising: a stent; and a plurality of layers on said stent; wherein at least one of said layers comprises a bioabsorbable polymer and at least one of said layers comprises a pharmaceutical agent selected from rapamycin, a prodrug, a derivative, an analog, a hydrate, an ester, and a salt thereof; wherein said device provides an in vitro pharmaceutical agent elution profile wherein said elution profile shows about 5% to about 25% of pharmaceutical agent is eluted one hour after the device is contacted with elution media; 5% to about 35% of pharmaceutical agent is eluted 3 hours after the device is contacted with elution media; about 30% to about 65% of pharmaceutical agent is eluted 1 day after the device is contacted with elution media; about 45% to about 70% of pharmaceutical agent is eluted 3 days after the device is contacted with elution media; about 55% to about 85% of pharmaceutical agent is eluted 10 days after the device is contacted with elution media about 65% to about 85% of pharmaceutical agent is eluted 15 days after the device is contacted with elution media; and about 75% to about 100% of pharmaceutical agent is eluted 20 days after the device is contacted with elution media.

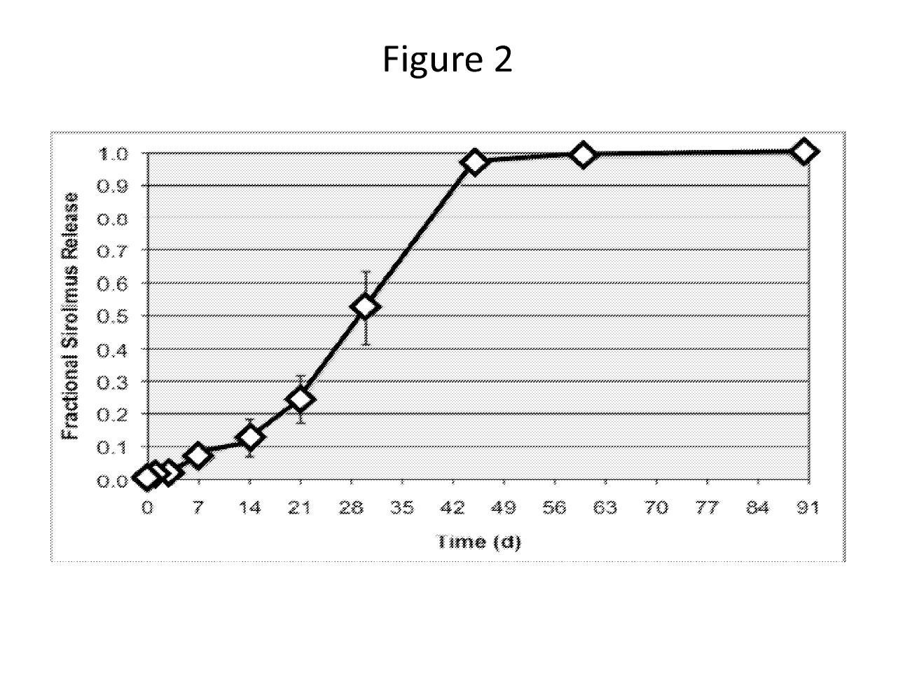

Provided herein is a device comprising: a stent; and a plurality of layers on said stent; wherein at least one of said layers comprises a bioabsorbable polymer and at least one of said layers comprises a pharmaceutical agent selected from rapamycin, a prodrug, a derivative, an analog, a hydrate, an ester, and a salt thereof; wherein said device provides an in vitro pharmaceutical agent elution profile shown in FIG. 2.

In some embodiments, the in vitro pharmaceutical agent elution profile is determined by a procedure comprising: (i) contacting the device with an elution media comprising ethanol and phosphate buffered saline wherein the pH of the media is about 7.4 and wherein the device is contacted with the elution media at a temperature of about 37° C.; (ii) optionally agitating the elution media during the contacting step in (i); (iii) removing the elution media at designated time points; and (iv) assaying the removed elution media to determine pharmaceutical agent content.

In some embodiments, the in vitro pharmaceutical agent elution profile is determined by a procedure comprising: (i) contacting the device with an elution media comprising ethanol and phosphate buffered saline wherein the pH of the media is about 7.4 and wherein the device is contacted with the elution media at a temperature of about 37° C.; (ii) optionally agitating the elution media during the contacting step in (i); (iii) removing said device from the elution media at designated time points; and (iv) assaying the elution media to determine pharmaceutical agent content.

In some embodiments, the in vitro pharmaceutical agent elution profile is determined in the absence of agitation.

In some embodiments, the procedure further comprises: (v) determining polymer weight loss by comparing the weight of the device before and after the contacting step and adjusting for the amount of pharmaceutical agent eluted into the elution media as determined in step iv. The device of claim 160 wherein step v shows at least 50% of polymer is released into the media after the device is contacted with the media for 90 days or more.

In some embodiments, step (v) shows at least 75% of polymer is released into the media after the device is contacted with the media for 90 days or more. In some embodiments, step (v) shows at least 85% of polymer is released into the media after the device is contacted with the media for 90 days or more. In some embodiments, step (v) shows at least 50% of polymer is released into the media after the device is contacted with the media for about 90 days. In some embodiments, step (v) shows at least 75% of polymer is released into the media after the device is contacted with the media for about 90 days. In some embodiments, step (v) shows at least 85% of polymer is released into the media after the device is contacted with the media for about 90 days. In some embodiments, step (v) shows at least 95% of polymer is released into the media after the device is contacted with the media for about 90 days. As used herein, the term “about” when referring to the media contact time can vary up to 1%, 5%, 10%, 20%, 25%, 6 hrs, 12 hrs, 24 hrs, 1 day, 2 days, 3 days, 5 days, or 7 days.

Provided herein is a device comprising: a stent; and a coating comprising a pharmaceutical agent selected from rapamycin, a prodrug, a derivative, ester and a salt thereof and a polymer wherein the coating has an initial pharmaceutical agent amount; wherein when said device is delivered in a body lumen of a subject the pharmaceutical agent is delivered in vessel wall tissue of the subject as follows: from about 0.1% to about 35% of the initial pharmaceutical agent amount is delivered in the subject's vessel wall tissue one week after the device is delivered in the subject's body; and from about 0.5% to about 50% of the initial pharmaceutical agent amount is delivered in the subject's vessel wall tissue two weeks after the device is delivered in the subject's body. As used herein, the term “about” when used in reference to percent delivery of the pharmaceutical agent means variations of any of 0.01%, 0.05%, 0.1%, 0.5%, 1%, 2%, 5%, 10%, 15%, 20%, 25%, 30%, and 50% on either side of the percent elution or on a single side of the aspect target, depending on the embodiment. For non-limiting example, for an delivery of 25% having a variation of 5%, this could mean 25% plus or minus 5%—equating to a range of 20% to 30%.

In some embodiments, the amount delivered to the subject's lumen is obtained by adding pharmaceutical agent present alone in said subject's vessel wall tissue and pharmaceutical agent delivered together with said polymer. In some embodiments, the subject is a human.

In some embodiments, subject is a pig and the amount of pharmaceutical agent delivered in the subject's vessel wall tissue is determined as follows: delivering the device in the pig's blood vessel lumen; euthanizing the pig at predetermined period of time after the device is delivered in the pig's blood vessel lumen and explanting the device; measuring the amount of pharmaceutical agent delivered in the vessel wall tissue. In some embodiments, subject is a rabbit and the amount of pharmaceutical agent delivered in the subject's vessel wall tissue is determined as follows: delivering the device in the rabbit's blood vessel lumen; euthanizing the rabbit at predetermined period of time after the device is delivered in the rabbit's blood vessel lumen and explanting the device; measuring the amount of pharmaceutical agent delivered in the vessel wall tissue.

Provided herein, a device comprising: a stent; and a coating comprising a pharmaceutical agent selected from rapamycin, a prodrug, a derivative, an analog, a hydrate, an ester, and a salt thereof and a bioabsorbable polymer wherein the coating has an initial pharmaceutical agent content of about 1 μg/mm to about 15 μg/mm; wherein said device provides an area under a curve (AUC) for content of pharmaceutical agent delivered in the vessel wall tissue of a subject over time as follows: from about 0.05 (m/mm)*day to about 1 (m/mm)*day when AUC is calculated from the time the device is delivered in a subject's body to one day after the device is delivered in the subject's body; from about 5 (m/mm)*day to about 10 (m/mm)*day when AUC is calculated starting after the first week the device is delivered in the subject's body through the second week after the device is delivered in the subject's body; from about 10 (m/mm)*day to about 20 (m/mm)*day when AUC is calculated starting after the second week the device is delivered in the subject's body through the fourth week after the device is delivered in the subject's body; and an AUClast of from about 40 (m/mm)*day to about 60 (m/mm)*day. As used herein, the term “about” when used in reference to AUC means variations of any of 0.01%, 0.05%, 0.1%, 0.5%, 1%, 2%, 5%, 10%, 15%, 20%, 25%, 30%, and 50% on either side of the target, depending on the embodiment.

Provided herein is a device comprising: a stent; and a coating comprising a pharmaceutical agent selected from rapamycin, a prodrug, a derivative, an analog, a hydrate, an ester, and a salt thereof and a bioabsorbable polymer wherein the coating has an initial polymer amount; wherein when said device is delivered in a body lumen of a subject about 75% of polymer is released from the device 90 days or more after the device is delivered in the body lumen of the subject. As used herein, the term “about” when used in reference to percent elution means variations of any of 0.01%, 0.05%, 0.1%, 0.5%, 1%, 2%, 5%, 10%, 15%, 20%, 25%, 30%, and 50% on either side of the percent elution or on a single side of the target, depending on the embodiment. For non-limiting example, for an elution of 25% having a variation of 5%, this could mean 25% plus or minus 5%—equating to a range of 20% to 30%.

Provided herein is a device comprising: a stent; and a coating comprising a pharmaceutical agent selected from rapamycin, a prodrug, a derivative, an analog, a hydrate, an ester, and a salt thereof and a bioabsorbable polymer wherein the coating has an initial polymer amount; wherein when said device is delivered in a body lumen of a subject about 85% of polymer is released from the device about 90 days after the device is delivered in the body lumen of the subject. As used herein, the term “about” when used in reference to percent elution means variations of any of 0.01%, 0.05%, 0.1%, 0.5%, 1%, 2%, 5%, 10%, 15%, 20%, 25%, 30%, and 50% on either side of the percent elution or on a single side of the target, depending on the embodiment. For non-limiting example, for an elution of 25% having a variation of 5%, this could mean 25% plus or minus 5%—equating to a range of 20% to 30%. As used herein, the term “about” when referring to the media contact time can vary up to 1%, 5%, 10%, 20%, 25%, 6 hrs, 12 hrs, 24 hrs, 1 day, 2 days, 3 days, 5 days, or 7 days.

Provided herein is a device comprising: a stent; and a coating comprising a pharmaceutical agent selected from rapamycin, a prodrug, a derivative, an analog, a hydrate, an ester, and a salt thereof and a bioabsorbable polymer wherein the coating has an initial polymer amount; wherein when said device is delivered in a body lumen of a subject at least about 75% of polymer is released from the device about 90 days after the device is delivered in the body lumen of the subject. As used herein, the term “about” when used in reference to percent elution means variations of any of 0.01%, 0.05%, 0.1%, 0.5%, 1%, 2%, 5%, 10%, 15%, 20%, 25%, 30%, and 50% on either side of the percent elution or on a single side of the aspect target, depending on the embodiment. For non-limiting example, for an elution of 25% having a variation of 5%, this could mean 25% plus or minus 5%—equating to a range of 20% to 30%. As used herein, the term “about” when referring to the media contact time can vary up to 1%, 5%, 10%, 20%, 25%, 6 hrs, 12 hrs, 24 hrs, 1 day, 2 days, 3 days, 5 days, or 7 days.

Provided herein is a device comprising: a stent; and a coating comprising a pharmaceutical agent selected from rapamycin, a prodrug, a derivative, an analog, a hydrate, an ester, and a salt thereof and a bioabsorbable polymer wherein the coating has an initial polymer amount; wherein when said device is delivered in a body lumen of a subject about 100% of polymer is released from the device about 90 days after the device is delivered in the body lumen of the subject. As used herein, the term “about” when used in reference to percent elution means variations of any of 0.01%, 0.05%, 0.1%, 0.5%, 1%, 2%, 5%, 10%, 15%, 20%, 25%, 30%, and 50% on either side of the percent elution or on a single side of the target, depending on the embodiment. For non-limiting example, for an elution of 25% having a variation of 5%, this could mean 25% plus or minus 5%—equating to a range of 20% to 30%. As used herein, the term “about” when referring to the media contact time can vary up to 1%, 5%, 10%, 20%, 25%, 6 hrs, 12 hrs, 24 hrs, 1 day, 2 days, 3 days, 5 days, or 7 days.

In some embodiments, the subject is a human. In some embodiments, the subject is a pig and the amount of polymer released from the device is determined as follows: delivering the device in the pig's blood vessel lumen; euthanizing the pig at predetermined period of time after the device is delivered in the pig's blood vessel lumen and explanting the device; and measuring the amount of polymer released from the device.

In some embodiments, measuring the amount of polymer released from the device comprises LC/MS/MS measurements. In some embodiments, measuring the amount released from the device comprises weight loss measurement. In some embodiments, weight loss measurement comprises measuring an amount of polymer remaining in the device and subtracting said remaining amount from the initial amount present in the device prior to delivering the device to the pig's blood vessel lumen.

Provided herein is a device comprising a stent; and a plurality of layers on said stent; wherein at least one of said layers comprises a bioabsorbable polymer and at least one of said layers comprises a pharmaceutical agent selected from rapamycin, a prodrug, a derivative, an analog, a hydrate, an ester, and a salt thereof, wherein the device has an initial pharmaceutical agent content of about 1 μg/mm to about 15 μg/mm; wherein when said device is delivered in a body lumen of a subject said device provides a blood concentration within 60 minutes from delivery of said device to the subject's body lumen that is from about 1% to about 50% of the blood concentration provided by a conventional drug eluting stent delivered to the subject under similar conditions. The term “about” when used in reference to a percent of blood concentration provided by a conventional drug eluting stent means variations of any of 0.01%, 0.05%, 0.1%, 0.5%, 1%, 2%, 5%, 10%, 15%, 20%, and 25% on either side of the percent or on a single side of the percent, depending on the embodiment. For non-limiting example, for a blood concentration that is 50% of the blood concentration provided by a conventional drug eluting stent and having a variability of 5%, the blood concentration may range from 45% to 55%, (i.e. 5% about the target of 50%).

Provided herein is a device comprising a stent; and a plurality of layers on said stent; wherein at least one of said layers comprises a bioabsorbable polymer and at least one of said layers comprises a pharmaceutical agent selected from rapamycin, a prodrug, a derivative, an analog, a hydrate, an ester, and a salt thereof, wherein the device has an initial pharmaceutical agent content of about 1 μg/mm to about 15 μg/mm; wherein when said device is delivered in a body lumen of a subject said device provides a blood concentration within 60 minutes from delivery of said device to the subject's body lumen that is from about 11% to about 20% of the blood concentration provided by a conventional drug eluting stent delivered to the subject under similar conditions.

Provided herein is a device comprising a stent; and coating on said stent; wherein said coating comprises a bioabsorbable polymer and a pharmaceutical agent selected from rapamycin, a prodrug, a derivative, an analog, a hydrate, an ester, and a salt thereof, wherein the device has an initial pharmaceutical agent content of about 1 μg/mm to about 15 μg/mm; wherein when said device is delivered in a body lumen of a subject said device provides about the same blood concentration over the first 72 hours from delivery of said device to the subject's body lumen.

In some embodiments, the blood concentration during the first 72 hours from delivery of said device to the subject's body lumen remains between 75% and 125% of an average blood concentration calculated over the first 72 hours from delivery of said device to the subject's body lumen. In some embodiments, the average blood concentration is from about 0.05 ng/mL to about 0.5 ng/mL. In some embodiments, the device provides an AUC for blood concentration over a period of 72 hours after the device is delivered to the subject's body lumen of from about 2 (ng/mL)*hour to about 20 (ng/mL)*hour.

In some embodiments, the device provides an AUC for blood concentration over a period of 72 hours after the device is delivered to the subject's body lumen of from about 4 (ng/mL)*hour to about 10 (ng/mL)*hour. In some embodiments, at least part of pharmaceutical agent is in crystalline form. In some embodiments, the pharmaceutical agent is provided at a reduced dose compared to a conventional drug eluting stent. In some embodiments, at least one of said layers comprises a PLGA bioabsorbable polymer.

In some embodiments, the pharmaceutical agent in said device has a shelf stability of at least 12 months.

In some embodiments, the device provides an in vitro pharmaceutical agent elution profile comparable to first order kinetics.

In some embodiments, the device provides pharmaceutical agent tissue concentration of at least twice the tissue concentration provided by a conventional stent. In some embodiments, the device provides a pharmaceutical agent tissue concentration of at least 5 times greater than the tissue concentration provided by a conventional stent. In some embodiments, the device provides a pharmaceutical agent tissue concentration of at least 25 times greater than the tissue concentration provided by a conventional stent. In some embodiments, the device provides a pharmaceutical agent tissue concentration of at least 100 times greater than the tissue concentration provided by a conventional stent.

In some embodiments, about 50% of said polymer is resorbed within 45-90 days after an angioplasty procedure wherein said device is delivered in a subject's body. In some embodiments, about 75% of said polymer is resorbed within 45-90 days after an angioplasty procedure wherein said device is delivered in a subject's body. In some embodiments, about 95% of said polymer is resorbed within 45-90 days after an angioplasty procedure wherein said device is delivered in a subject's body. The term “about” when referring to the percent of the polymer resorbed means variations of any of 0.01%, 0.05%, 0.1%, 0.5%, 1%, 2%, 5%, 10%, 15%, 20%, and 25% on either side of the percent or on a single side of the percent.

In some embodiments, 99% of said polymer is resorbed within 45-90 days after an angioplasty procedure wherein said device is delivered in a subject's body. In some embodiments, at least 99% of said polymer is resorbed within 45-90 days after an angioplasty procedure wherein said device is delivered in a subject's body. In some embodiments, at least 95% of said polymer is resorbed within 45-90 days after an angioplasty procedure wherein said device is delivered in a subject's body. In some embodiments, at least 90% of said polymer is resorbed within 45-90 days after an angioplasty procedure wherein said device is delivered in a subject's body. In some embodiments, at least 80% of said polymer is resorbed within 45-90 days after an angioplasty procedure wherein said device is delivered in a subject's body. In some embodiments, at least 75% of said polymer is resorbed within 45-90 days after an angioplasty procedure wherein said device is delivered in a subject's body. In some embodiments, 100% of said polymer is resorbed within 45-90 days after an angioplasty procedure wherein said device is delivered in a subject's body. In some embodiments, at least 99% of said polymer is absorbed within 45-90 days after an angioplasty procedure wherein said device is delivered in a subject's body. In some embodiments, at least 95% of said polymer is absorbed within 45-90 days after an angioplasty procedure wherein said device is delivered in a subject's body. In some embodiments, at least 90% of said polymer is absorbed within 45-90 days after an angioplasty procedure wherein said device is delivered in a subject's body. In some embodiments, at least 80% of said polymer is absorbed within 45-90 days after an angioplasty procedure wherein said device is delivered in a subject's body. In some embodiments, at least 75% of said polymer is absorbed within 45-90 days after an angioplasty procedure wherein said device is delivered in a subject's body. In some embodiments, 100% of said polymer is absorbed within 45-90 days after an angioplasty procedure wherein said device is delivered in a subject's body.

Generally, polymers associated with drug-eluting stents are solvent labile and susceptible to artifactual removal during the histologic processing of implanted arteries. Polymers, present during the formation of the neointima, are a space occupying mass in which the smooth muscle cells must accommodate, and around which the nascent neointima must form. With the removal of the polymer during processing, clear spaces are created and interpreted to be the negative image, or approximate facsimile, of the stent polymer/drug in situ, despite the absence of observable polymer. As such, these clear areas may be used to qualitatively characterize the size, spread, localization and apparent resorption of polymer coating material as a function of implant duration. Bioabsorbable polymers will resorb over time by the body, thus, over time these clear areas will be fewer and smaller, until the polymer is fully absorbed (resorbed). These may be detected by histologic processing of implanted arteries, such as noted in Examples 34-37, at least, and visualized under microscopy as noted therein.

The polymer/drug coating of the coated stents described herein are typically characterized by a larger clear zone intimately surrounding the struts. Lacunae refers to variably sized and shaped clear space(s) located in the peri/extra-strut neointima which appear to have been separated from the polymer intimately associated with the struts. These lacunae were interpreted to represent the deposition/migration of the strut-associated polymer/drug into the surrounding neointima. Since lacunae were not observed in the bare metal stents similarly implanted, their presence in the coated stent tissue samples may be the local effects of neointimal formation inhibition secondary to the presence of the polymer (i.e., space-occupying mass) and/or sirolimus (i.e., smooth muscle cell inhibition).

Neointimal lacunae were only observed only at Day 30 (when evaluated at about day 3, 30, 90, 180, and 365), whether there was a single coated stent implanted, or whether there were two coated stents implanted (overlapping as noted elsewhere herein). The magnitude of extra-strut neointimal lacunae (i.e., polymer/drug) was minimal, and though they were commonly seen on a per plane basis (˜70%), within each affected plane the change was generally limited to only one to two foci. Regardless, the presence of neointimal lacunae after 30 days implantation of the coated stents implanted as noted herein did not appear to be associated with any adverse tissue response. Rarely (˜<5%), lacunae were present in the adventitia, with associated inflammation, and usually the result of mural injury.

In some embodiments, the device provides reduced inflammation over the course of polymer resorbtion compared to a conventional stent.

Provided herein is a method of treating a subject comprising delivering a device as described herein in a body lumen.

Provided herein, is a method of treating a subject comprising delivering in the body of the subject a device comprising: a stent; and a coating comprising a pharmaceutical agent selected from rapamycin, a prodrug, a derivative, an analog, a hydrate, an ester, and a salt thereof and a polymer wherein the coating has an initial pharmaceutical agent amount; wherein said device is delivered in a body lumen of the subject and the pharmaceutical agent is delivered in vessel wall tissue of the subject as follows: i. from about 0.05% to about 35% of the initial pharmaceutical agent amount is delivered in the subject's vessel wall tissue one week after the device is delivered in the subject's body; and ii. from about 0.5% to about 50% of the initial pharmaceutical agent amount is delivered in the subject's vessel wall tissue two weeks after the device is delivered in the subject's body.

In some embodiments, the device provides reduced inflammation over the course of polymer resorbtion.

In some embodiments, the presence of crystallinity is shown by at least one of XRD, Raman Spectroscopy, Infrared analytical methods, and DSC.

In some embodiments, the coating on an abluminal surface of said stent has a greater thickness than coating on a luminal surface of said stent. In some embodiments, the ratio of coating on the abluminal surface to coating on the luminal surface of the device is 80:20. In some embodiments, the ratio of coating on the abluminal surface to coating on the luminal surface of the device is 75:25. In some embodiments, the ratio of coating on the abluminal surface to coating on the luminal surface of the device is 70:30. In some embodiments, the ratio of coating on the abluminal surface to coating on the luminal surface of the device is 60:40.

Provided herein is a device comprising a stent comprising a cobalt-chromium alloy; and a coating on the stent; wherein the coating comprises at least one polymer and at least one active agent; wherein at least one of: quantified neointima, media, percent stenosis, wall injury, and inflammation exhibited at 30 days following implantation of the device in a first artery of an animal is significantly reduced for the device as compared to a bare metal cobalt-chromium stent implanted in a second artery of an animal when both the device and the bare metal cobalt chromium stent are compared in a the study, wherein the study design overlaps two of the devices in the first artery and overlaps two of the bare metal cobalt-chromium stents in the second artery.

In some embodiments, the test performed to determine significant differences between the device and the bare metal cobalt-chromium stent is the Mann-Whitney Rank Sum Test and the p value is less than 0.10. In some embodiments, the test performed to determine significant differences between the device and the bare metal cobalt-chromium stent is the Mann-Whitney Rank Sum Test and the p value is less than 0.05.

In some embodiments, at least one of wall injury, inflammation, neointimal maturation, and adventitial fibrosis of the device tested at day 3 of the animal study is equivalent to the bare metal stent.

In some embodiments, at least one of lumen area, artery area, lumen diameter, IEL diameter, stent diameter, arterial diameter, lumen area/artery area ratio, neointimal area/medial area ratio, EEL/IEL ratio, endothelialization, neointimal maturation, and adventitial fibrosis of the device tested at day 30 of the animal study is equivalent to the bare metal stent.

In some embodiments, at least one of lumen area, artery area, neointimal area, medial area, percent stenosis, wall injury, and inflammation of the device tested at day 30 of the animal study is equivalent to the bare metal stent.

In some embodiments, at least one of lumen area, artery area, neointimal area, medial area, percent stenosis, wall injury, inflammation, endothelialization, neointimal maturation, and adventitial fibrosis of the device tested at day 30 of the animal study is equivalent to the bare metal stent.

In some embodiments, the active agent is at least one of: 50% crystalline, at least 75% crystalline, at least 90% crystalline.

In some embodiments, the polymer comprises a bioabsorbable polymer. In some embodiments, the polymer comprises PLGA. In some embodiments, the polymer comprises PLGA with a ratio of about 40:60 to about 60:40 and further comprises PLGA with a ratio of about 60:40 to about 90:10. In some embodiments, the polymer comprises PLGA having a molecular weight of about 10 kD (weight average molecular weight) and wherein the coating further comprises PLGA having a molecular weight of about 19 kD (weight average molecular weight). In some embodiments, the polymer is selected from the group: PLGA, a copolymer comprising PLGA (i.e. a PLGA copolymer), a PLGA copolymer with a ratio of about 40:60 to about 60:40, a PLGA copolymer with a ratio of about 70:30 to about 90:10, a PLGA copolymer having a molecular weight of about 10 kD (weight average molecular weight), a PLGA copolymer having a molecular weight of about 19 kD (weight average molecular weight), a PLGA copolymer having a number average molecular weight of between about 9.5 kD and about 25 kD, a PLGA copolymer having a number average molecular weight of between about 14.5 kD and about 15 kD, PGA poly(glycolide), LPLA poly(l-lactide), DLPLA poly(dl-lactide), PCL poly(e-caprolactone) PDO, poly(dioxolane) PGA-TMC, 85/15 DLPLG p(dl-lactide-co-glycolide), 75/25 DLPLG, 65/35 DLPLG, 50/50 DLPLG, TMC poly(trimethylcarbonate), poly(anhydrides) such as p(CPP:SA) poly(1,3-bis-p-(carboxyphenoxy)propane-co-sebacic acid), and a combination thereof. As used herein, the term “about,” when referring to a copolymer ratio, means variations of any of 0.5%, 1%, 2%, 5%, 10%, 15%, 20%, 25%, 30%, and 50%, depending on the embodiment. For example, a copolymer ratio of 40:60 having a variation of 10% ranges from 35:65 to 45:55, which is a range of 10% of the total (100) about the target. As used herein, the term “about” when referring to a polymer molecular weight means variations of any of 0.5%, 1%, 2%, 5%, 10%, 15%, 20%, 25%, 30%, and 50%, depending on the embodiment. For example, a polymer molecular weight of 10 kD (weight average molecular weight) having a variation of 10% ranges from 9 kD to 11 kD, which is a range of 10% of the target 10 kD on either side of the target 10 kD.

In some embodiments, the stent is formed from a material comprising the following percentages by weight: about 0.05 to about 0.15 C, about 1.00 to about 2.00 Mn, about 0.04 Si, about 0.03 P, about 0.3 S, about 19.0 to about 21.0 Cr, about 9.0 to about 11.0 Ni, about 14.0 to about 16.00 W, about 3.0 Fe, and Bal. Co. In some embodiments, the stent is formed from a material comprising at most the following percentages by weight: about 0.025 C, about 0.15 Mn, about 0.15 Si, about 0.015 P, about 0.01 S, about 19.0 to about 21.0 Cr, about 33 to about 37 Ni, about 9.0 to about 10.5 Mo, about 1.0 Fe, about 1.0 Ti, and Bal. Co. As used herein, the term “about,” when referring to a weight percentage of stent material, means variations of any of 0.5%, 1%, 2%, 5%, 10%, 15%, 20%, 25%, 30%, and 50% of the total weight percent (i.e. 100%) on either side (+/−) of the weight percentage, depending on the embodiment. For example, a weight percentage of stent material of 3.0 Fe having a variation of 1% ranges from 2.0 to 4.0, which is a range of 1% of the total (100) on either side of the target 3.0.

In some embodiments, the stent has a thickness of from about 50% to about 90% of a total thickness of the device. In some embodiments, the coating has a total thickness of from about 5 μm to about 50 μm. The coating can be conformal around the struts, isolated on the abluminal side, patterned, or otherwise optimized for the target tissue. As used herein, the term “about” when referring to a device thickness or coating thickness means variations of any of 0.5%, 1%, 2%, 5%, 10%, 15%, 20%, 25%, 30%, and 50%, depending on the embodiment. For non-limiting example, a device thickness of 20 μm having a variation of 10% ranges from 18 μm to 22 μm, which is a range of 10% on either side of the target 20 μm. For non-limiting example, a coating thickness of 100 μm having a variation of 10% ranges from 90 μm to 110 μm, which is a range of 10% on either side of the target 100 μm.

In some embodiments, the device has an active agent content of from about 5 μg to about 500 μg. In some embodiments, device has an active agent content of from about 100 μg to about 160 μg. As used herein, the term “about” when referring to a pharmaceutical agent content means variations of any of 0.5%, 1%, 2%, 5%, 10%, 15%, 20%, 25%, 30%, and 50%, depending on the embodiment. For non-limiting example, a pharmaceutical agent content of 120 μg having a variation of 10% ranges from 108 μg to 132 μg, which is a range of 10% on either side of the target 120 μg. Where content is expressed herein in units of μg/mm, however, this may simply be converted to μg/mm2 or another amount per area (e.g., μg/cm2), or vice versa, or converted to a total pharmaceutical content by multiplying by the area or length as needed.

In some embodiments, the active agent comprises a macrolide immunosuppressive (limus) drug. In some embodiments, the macrolide immunosuppressive drug comprises one or more of: rapamycin, biolimus (biolimus A9), 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′:E,4′S)-40-O-(4′,5′-Dihydroxypent-2′-en-1′-yl)-rapamycin, 40-O-(2-Hydroxy)ethoxycar-bonylmethyl-rapamycin, 40-O-(3-Hydroxyl)propyl-rapamycin, 40-O-(6-Hydroxyl)hexyl-rapamycin, 40-O-[2-(2-Hydroxyl)ethoxy]ethyl-rapamycin, 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin 40-O-(2-Nicotinoyloxy)ethyl-rapamycin, 40-O-[2-(N-Morpholino)acetoxy]ethyl-rapamycin, 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxyl)ethyl-rapamycin, 28-O-Methyl-rapamycin, 40-O-(2-Aminoethyl)-rapamycin, 40-O-(2-Acetaminoethyl)-rapamycin, 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl)rapamycin (tacrolimus), 42-[3-hydroxy-2-(hydroxymethyl)-2-methylpropanoate]rapamycin (temsirolimus), (42S)-42-Deoxy-42-(1H-tetrazol-1-yl)-rapamycin (zotarolimus), and salts, derivatives, isomers, racemates, diastereoisomers, prodrugs, hydrate, ester, or analogs thereof. In some embodiments, the macrolide immunosuppressive drug comprises a polymorph of any of the macrolide immunosuppressive drugs noted herein and/or any other macrolide immunosuppressive drug.

### EXAMPLES

The following examples are provided to illustrate selected embodiments. They should not be considered as limiting the scope of the invention, but merely as being illustrative and representative thereof. For each example listed below, multiple analytical techniques may be provided. Any single technique of the multiple techniques listed may be sufficient to show the parameter and/or characteristic being tested, or any combination of techniques may be used to show such parameter and/or characteristic. Those skilled in the art will be familiar with a wide range of analytical techniques for the characterization of drug/polymer coatings. Techniques presented here, but not limited to, may be used to additionally and/or alternatively characterize specific properties of the coatings with variations and adjustments employed which would be obvious to those skilled in the art.

### Example 1

Stents, mounted on holders, supported on a carousel may be introduced into a coating chamber. The process comprises providing a cloud of charged particles to the stents that are orbiting through the cloud. For the polymer coating steps, this is accomplished by the rapid expansion of the pressurized solution of polymer in densified 1,1,1,2,3,3-hexafluoropropane (FC-236EA) through a small diameter stainless steel orifice. Heat is applied to the orifice to overcome Joule-Thompson cooling and to ensure that the compressed gas is fully vaporized on expansion from the orifice. Further control of the charged polymer cloud is obtained through controlling the polymer solution concentration and flow rate. Flow rate is controlled implicitly by the pressure drop across the nozzle from a constant pressure polymer solution provided by an automated syringe pump.

The solution concentration is controlled by the mass of polymer added to the dissolving chamber and the volume filled within the automated syringe pump. The concentration may be 2 w/v %, 4 w/v %, about 2 w/v %, about 4 w/v %, about 2 w/v % to about 4 w/v %, 2 w/v % to 4 w/v %, 2 w/v %+/−0.5 w/v %, 2 w/v %+/−0.25 w/v %, 2 w/v %+/−0.1 w/v %, 4 w/v %+/−0.5 w/v %, 4 w/v %+/−0.25 w/v %, 4 w/v %+/−0.1 w/v %, at least 1 w/v %, at least 1.5 w/v %, at least 2 w/v %, at least 3 w/v %, at least 4 w/v %, at most 4 w/v %, at most 5 w/v %, at most 6 w/v %, at most 7 w/v %, at most 8 w/v %, at most 9 w/v %, at most 10 w/v %, at most 11 w/v %, at most 12 w/v %, at most 13 w/v %, at most 14 w/v %, or at most 15 w/v %. depending on the embodiment. An increase in polymer concentration may coincide with in an attendant decrease in polymer spray time (at fixed flow rate) and greater capacity to coat multiple carousels from a single polymer solution—increasing throughput. Both 2% and 4% have been tested, as examples, and provide a similar polymer particle cloud to the stents. Concentrations of up to 5% have been used yet the upper bound of concentration usable in the coating process has not been determined. In addition, because the sintering steps following application of the polymer particles change the primary polymer particle morphology (but not that of the active agent—whether it be a pharmaceutical agent or biologic agent), implementing the increased solution concentration results in a coating with the same content and performance properties as those coated from 2 w/v % solutions. The limiting effect of concentration is to be low enough to provide particles, instead of fibers, at the exit of the nozzle, wherein particulate may be defined by an aspect ratio of less than 2:1.

Multiple polymer sprays may be incorporated in the coating process (e.g. 2× sprays in the second application of polymer, 3× sprays in the final application of polymer) to provide a consistent cloud and electrostatic environment for all polymer applications, while providing more polymer in the subsequent layers.

A contributing factor to stent coating is the creation of a uniform and reproducible electric potential between the particles (active agent or PLGA), stent and surrounding components (carousel, platform, stainless steel covers, etc.). The process is based on the electrical principle of opposites attract & likes repel. In the process, opposite polarities on the stents vs. particles are established to create an electric field that attracts the particles to the stent. Additionally, the particles are polarized the same as the internal surfaces of the coating chamber leading to enhanced deposition on the stent. Increasing the voltage difference between the stents and the coating chamber increases coating efficiency. However, as voltage increases, the stent struts and fine wires of the stent holder can generate corona discharge disturbing the electric field that can result in poor drug coating consistency. A variety of potentials may be used, however in the present example potentials of ±1.5 kV are conservatively used to minimize the risk of corona discharge and its deleterious effects on the coating. In other embodiments, example potentials include, but are not limited to: ±1.0 kV, ±1.2 kV, ±1.3 kV, ±1.4 kV, ±1.5 kV, ±1.6 kV, ±1.7 kV, ±1.8 kV, ±1.9 kV, ±2 kV, ±3 kV, ±3.5 kV, ±4 kV, ±5 kV, from ±1.0 kV to ±2.0 kV, from ±1.2 kV to ±1.8 kV, from ±1.4 kV to ±1.6 kV, from ±0.5 kV to ±5 kV, or about ±1.5 kV.

After application of each polymer coating step, the stents may be sintered to coalesce the powder coating into a smooth film encapsulating the stent's struts. To accomplish this sintering, the individual stents mounted on stent holders may be moved from the coating carousel into an isothermal sintering chamber set at >40° C. in the instance of PLGA, or any temperature appropriate for the polymer and drug in question—i.e. at or around the Tg (glass transition temperature) of the polymer, but below the temperature at which the drug (active agent) would change its morphology and/or change its activity. The Tg of PLGA used in this example (50:50), is about 45° C., thus, a setting of >40° C. is sufficient to sinter. A temperature setting at or near the Tg may require longer sintering time. In some embodiments, the sintering temperature is 100° C., or about 100° C.

The transfer of the stents from the coating carousel to the isothermal sintering chamber is performed touching only the stent holder wire-form because the coating prior to sintering is a powder held only by electrostatic image charge. The sintering may be accomplished by exposing the stent to conditions sufficient to coalesce the powder, yet not aggressive enough to alter the crystalline particle morphology of the drug (or activity of the biologic agent) or cause degradation of the polymer or active agent. For example the setting may be >40° C. in the instance of PLGA, or any temperature appropriate for the polymer and drug in question—i.e. at or around the Tg (glass transition temperature) of the polymer, but below the temperature at which the drug (active agent) would change its morphology and/or change its activity. The Tg of PLGA used in this example (50:50), is about 45° C., thus, a setting of >40° C. is sufficient to sinter. A temperature setting at or near the Tg may require longer sintering time. In some embodiments, the sintering temperature is 100° C., or about 100° C. Conditions that would degrade the coating are, for example, solvents, solvent vapors or temperatures >150° C. (in the instance of PLGA and rapamycin). Other temperatures for other polymers and drugs would be appropriate based on the principles noted herein to retain the morphology and/or activity of the agents, and yet to meet or exceed the approximate Tg of the polymer.

Use of compressed gases provides benign conditions for the sintering of polymer powders on surfaces. This may include the use of certain gas in the sintering process, or merely elevated temperature and/or pressure. FC-236EA gas pressure may be provided at each sintering step, or only certain sintering steps. Use of the FC-236EA prolongs the coating process cycle time as compared to elevated temperature and/or pressure only. In certain embodiments, use of the FC-236EA pressurized gas in the sintering process is only at the final sintering step. Inadequate sintering conditions (temperature and/or pressure) can result in inconsistent stent topography and in vitro drug release kinetics.

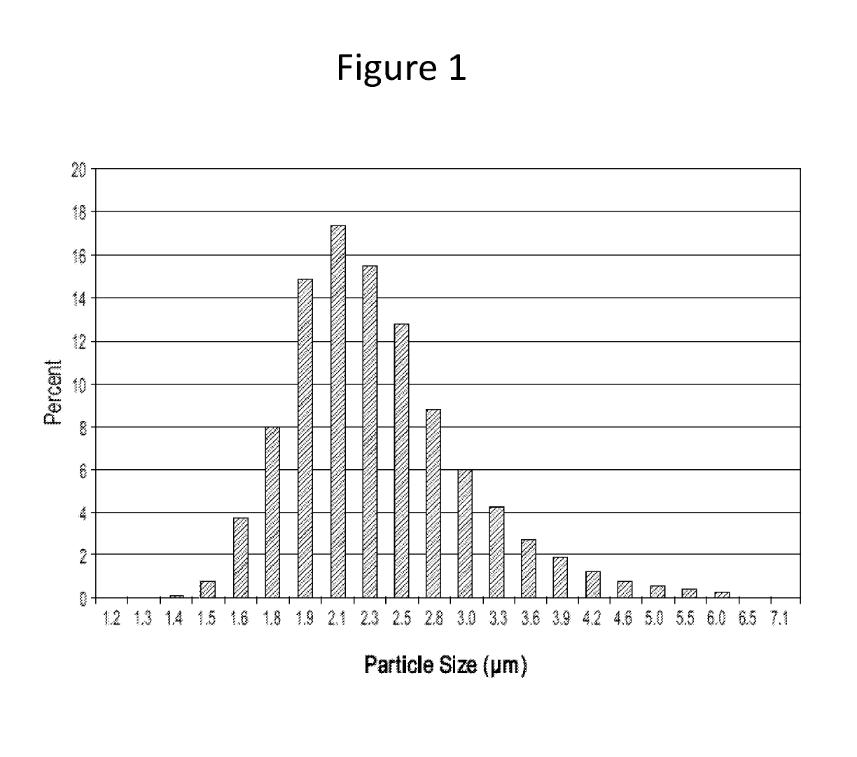

Sirolimus (or any powder form active agent, as noted herein), may be deposited on the stents by an electrostatic dry-powder process, but is distinguished from the polymer coating (e.g. PLGA) process in that the drug is never dissolved in a compressed gas or other solvent In some embodiments, the active agent is micronized prior to deposition on the stent. For example, raw sirolimus may be first micronized to achieve a particle distribution such that at least 99% by volume of the particles are less than 10 microns with the distribution centered at 2.75+/−0.5 microns. In other examples, the active agent may be first micronized to achieve a particle distribution such that 80%, 85%, 90%, 95%, 99%, at least 50%, at least 75%, at least 80%, at least 85%, at least 90%, at least 95%, at least 99%, at least about 50%, at least about 75%, at least about 80%, at least about 85%, at least about 90%, at least about 95%, or at least about 99% by volume of the particles are less than 3 microns, less than 5 microns, less than 7.5 microns, less than 10 microns, less than 20 microns, less than 25 microns, less than 30 microns, less than 40 microns, less than 50 microns, less than 75 microns, less than about 10 microns, less than about 15 microns, or less than about 7.5 microns, with the distribution centered at 1.0+/−0.5 microns, 1.25+/−0.5 microns, 1.5+/−0.5 microns, 1.75+/−0.5 microns, 2.0+/−0.5 microns, 2.25+/−0.5 microns, 2.5+/−0.5 microns, 2.75+/−0.5 microns, 3.0+/−0.5 microns, 3.25+/−0.5 microns, 3.5+/−0.5 microns, 3.75+/−0.5 microns, 4.0+/−0.5 microns, 4.25+/−0.5 microns, 4.5+/−0.5 microns, 4.75+/−0.5 microns, 5+/−0.5 microns, 5.5+/−0.5 microns, 6+/−0.5 microns, 6.5+/−0.5 microns, 7+/−0.5 microns, 7.5+/−0.5 microns, 8+/−0.5 microns, 8.5+/−0.5 microns, 9+/−0.5 microns, 10+/−0.5 microns, 15+/−0.5 microns, 20+/−0.5 microns, 25+/−0.5 microns, 30+/−0.5 microns, 35+/−0.5 microns, 40+/−0.5 microns, 45+/−0.5 microns, 50+/−0.5 microns, about 1.0 microns, about 1.5 microns, about 2.0 microns, about 2.5 microns, about 2.75 microns, about 3.0 microns, about 3.5 microns, about 4.0 microns, about 4.5 microns, about 5 microns, about 6 microns, about 7 microns, about 8 microns, about 9 microns, about 10 microns, about 15 microns, about 20 microns, about 25 microns, about 30 microns, about 35 microns, about 40 microns, about 45 microns, or about 50 microns. FIG. 1 depicts an embodiment of micronized sirolimus used in a spray coating process described in this Example, having a particle distribution such that at least 99% by volume of the particles are less than 10 microns with the distribution centered at 2.75+/−0.5 microns.

To provide a well dispersed cloud of the fine pharmaceutical agent particles, the drug may be pulsed in to the chamber using a fixed volume of nitrogen pressurized at 300 psi as the propellant. Depending on the embodiment, other propellants may be used, and other pressures. For example, any compatible non-reactive propellant may be used based on the active agent and/or the polymer being used, including but not limited to air, one or more noble gas (e.g. argon, nitrogen, helium), or any combination thereof. A variety of pulse pressures may be used, depending on the embodiment. For example, an operating pressure of at least 50 psi, at least 75 psi, at least 100 psi, at least 150 psi, at least 200 psi, at least 250 psi, at least 300 psi, about 50 psi, about 75 psi, about 100 psi, about 150 psi, about 200 psi, about 250 psi, about 300 psi, about 350 psi, about 400 psi, about 450 psi, about 500 psi, about 550 psi, about 600 psi, 50 psi to 500 psi, 200 psi to 400 psi, 250 psi to 350 psi, 50 psi, 75 psi, 100 psi, 150 psi, 200 psi, 250 psi, 300 psi, 350 psi, 400 psi, 450 psi, 500 psi, 550 psi, or 600 psi, may be used. In the instance of sirolimus sprayed as noted herein the operating pressure of 300 psi met the need to deliver a finely dispersed particle cloud into the coating chamber without adding complexity to the coating equipment.

While the stents are sintering, the coating carousel and chambers may be wiped with an acetone-moistened clean room cloth to prevent polymer or drug from building up over multiple cycles on the surfaces exposed to the electrostatic environment.

After the completion of the entire coating sequence: Polymer (1 spray)-Sinter (100° C., ambient pressure)-Drug-Polymer (2 sprays)-Sinter (100° C., ambient pressure)-Drug-Polymer (3 sprays)-Sinter (100° C., 150 psi pressurization with gaseous FC236ea), the stents may be removed from the stent holders for analysis and mounting on a catheter.

The parameters of active agent amount and polymer spray time are dependent on the size of stent (or other device) to be coated. During the course of production, total mass and agent content may be monitored.

### Example 2

Delayed healing, stent thrombosis, late-catch-up and neo-atherosclerosis are unfavorable late-term outcomes associated in part with permanent polymers of current drug-eluting stents (DES). Certain devices as described and produced according to methods herein were used in this example and were designed for improved safety while maintaining strong efficacy.

The devices used in this example comprised crystalline sirolimus and sirolimus release at a controlled, linear rate, with coating off the stent in 45-60 days and with full absorption of the polymer by 90 days. The coatings are lubricious and hydrophilic. The sirolimus eluting stent systems (Sirolimus DES and systems) were built according to methods described herein, and the coated stents comprised sirolimus and PLGA. The process for making the Sirolimus DES included supercritical fluid deposition which allowed the drug/polymer coating to be applied to a bare metal stent. The absorbable drug/polymer formulation controls drug elution and the duration of polymer exposure. As a result, the coating delivers a therapeutic solution for coronary artery disease with the potential to avoid the long-term safety concerns associated with current drug-eluting coronary stents that use non-absorbing or very slowly absorbing polymers. The sirolimus eluting stents (Sirolimus DES) comprised 3.0×15 mm CoCr stents, having a nominal drug dose per stent of 135 micrograms of sirolimus. Sirolimus DES stents were coated as follows: PDPDP layers (i.e. Polymer Drug Polymer Drug Polymer), having a sinter step (100° C./150 psi/10 min) after each “P” (or polymer) layer, wherein the polymer is 50:50 PLGA. There was 135 micrograms+/−15% sirolimus on each coated stent in this study. The coating was about 5-15 micrometers thick on each stent, and comprised a thicker coating on the abluminal surface (coating bias). The coating encapsuled each of the stents.

A human clinical trial was performed as a prospective, single-arm study at five sites evaluating the device as described herein and according to methods described herein for preliminary safety and efficacy. Patients with discrete de novo lesions (2.5-3.5 mm diameter and ≦20 mm length) in native coronary arteries were enrolled. The Patient selection criteria included stable or unstable angina pectoris (Class I, II, III, or IV), documented ischemia, or documented silent ischemia. Patients could not have recent Q wave MI (<72 hrs) and no elevated cardiac biomarkers. The target lesions included planned single, de novo, types A, B1 or B2 coronary lesions (according to the ACC/AHA classification) in the native coronary artery with >50% diameter stenosis. Vessel diameters were to be 2.5 to 3.5 mm and a maximum of a 23 mm long stent was used and indicated. Lesions were to be excluded if they were highly calcified, tortuous, thrombus present, or proximal angulation. Lesions were to be excluded if they were located at size branch over 2.5 mm, at an ostial location, or at a previously treated vessel. Non-target lesions were allowed to be treated if critical in another vessel prior to treating the target lesion.

DAPT (Dual Antiplatelet Therapy) recommendations were in accordance with ACC/AHA/SCAI PCI Practice guidelines. 100% of the patients were found to be taking aspirin daily at 8 months; 80% of the patients remained on daily Plavix of 75 mg at 8 months; 20% discontinued Plavix at 6 months.

Table 1 shows the Clinical and Lesion Characteristics of patients in this study (n=30).

TABLE 1
Baseline Clinical & Lesion Characteristics

| | | Actual | Percentages |
|---|---|---|---|
| Clinical Characteristics | | | |
| Demographics | Age (mean, range) | 62.3, 44-86 | NA |
| | Gender (m/f) | 22/8 | 73%/27% |
| Risk Factors | MI | 5 | 17% |
| | PCI | 9 | 30% |
| | CVA | 1 | 3% |
| | Hypertension | 23 | 77% |
| | Hypercholesterolemia | 26 | 87% |
| | Diabetes | 7 | 23% |
| Lesion Characteristics | | | |
| Baseline | Length | 13.15 +/− 3.77 | NA |
| Angiography | RVD | 2.86 +/− 0.32 | NA |
| Location | RCA | 12 | 40% |
| | LAD | 11 | 37% |
| | LCx | 7 | 23% |
| Classiciation | A | 16 | 53% |
| | B1 | 10 | 44% |
| | C | 4 | 13% |
| Tortuous | Moderate | 2 | 7% |
| Calcium | Moderate | 7 | 23% |
| | Severe | 1 | 3% |

Following the device placement, patients were evenly assigned to 3 groups for follow-up imaging (angiography, intravascular ultrasound evaluation (IVUS) and optical coherence tomography (OCT)) at 4-months, 6-months or 8-months to determine in-stent late lumen loss (LLL), vessel healing, and stent coverage. Clinical safety was conducted for all 30 patients at 8-months.

The initial 10 patients evaluated at 4-months demonstrated an in-stent late lumen loss (LLL) of 0.01±0.12 mm assessed by core laboratory quantitative angiography. Imaging with OCT demonstrated thin, homogenous coverage with high rates of stent strut coverage (70% of the patients having >90% strut coverage and 90% having >80% strut coverage) with a low rate of stent strut malapposition. The IVUS findings supported minimal neointimal hyperplasia with a neointimal obstruction of 5.2%. Clinical outcomes and angiographic, OCT and IVUS analyses at 4, 6 and 8-months for the entire study cohort will be presented, and is described further in Examples 3 and 4.

The device as described and tested according to this example, developed with a predictably absorbed polymer encompassing a new morphology of sirolimus, may address current DES concerns while being very effective. Interim imaging with angiography, OCT and IVUS suggest effective inhibition of neointimal hyperplasia with a high rate of strut coverage.

The coated stent as described and tested according to this example is unique as compared to other DES. As the coating migrates off the stent and is reabsorbed into the surrounding tissue, the crystalline sirolimus is deposited into the tissue and drug is released at a controlled rate. The coating is off the stent in 45-60 days leaving a bare metal stent (BMS) with complete absorption of the polymer by 90 days leaving. This methodology allows for managed drug delivery to the treated artery to reduce the extent of restenosis while allowing progressive stent strut coverage which can mean a reduced rate of late stent thrombosis as compared to other DES.

In some embodiments, a bare metal stent is achieved by 90 days at most, as demonstrated by full absorption of the polymer by the arterial tissue. In some embodiments, a bare metal stent is achieved by 45-60 days at most, as demonstrated by clearance of the polymer from the struts of the stent. Full absorption may be at least 75% absorption, at least 80% absorption, at least 90% absorption, at least 95% absorption, at least 99%, or 100% absorption as measured according to polymer detection methods described herein or known to one of skill in the art, such as methods of U.S. application Ser. No. 13/445,723, at least for histological evaluation in porcine models and evaluation by microscopy.

Absorption may also be referred to herein as resorption. Absorption (or resorption) of the polymer by arterial tissue (or by the vessel, or by the tissue surrounding the stent) may occur not only be actual absorption by such tissue, but may alternatively or additionally occur by resorbtion over time by the body in general, which can include metabolization and/or excretion of any part of the polymer or product of the polymer absorption process. For example in the case of PLGA absorption occurs by, hydrolysis of the PLGA to a low molecular weight whereby the degraded PLGA oligomers are soluble in body fluids, diffuse into the surrounding tissues and/or blood stream and are then either metabolized or excreted. Depending on the polymer used, other methods of absorption or resorption may exist and are considered covered by the idea of absorption or resorption of the polymer by arterial tissue, by the body, by the vessel, by the tissue surrounding the stent, or any variation thereof.

In order to determine whether absorption or resorption has occurred, and to what degree, it is generally understood that polymers associated with drug-eluting stents are solvent labile and susceptible to artifactual removal during the histologic processing of implanted arteries. Polymers, present during the formation of the neointima, are a space occupying mass in which the smooth muscle cells must accommodate, and around which the nascent neointima must form. With the removal of the polymer during processing, clear spaces are created and interpreted to be the negative image, or approximate facsimile, of the stent polymer/drug in situ, despite the absence of observable polymer. As such, these clear areas may be used to qualitatively characterize the size, spread, localization and apparent resorption of polymer coating material as a function of implant duration. Bioabsorbable polymers will resorb over time by the body, thus, over time these clear areas will be fewer and smaller, until the polymer is fully absorbed (resorbed). These may be detected by histologic processing of implanted arteries, such as methods of U.S. application Ser. No. 13/445,723, at least, and visualized under microscopy as noted therein.

The polymer and drug coating of the coated stents described herein are typically characterized by a larger clear zone intimately surrounding the struts. Lacunae refers to variably sized and shaped clear space(s) located in the peri/extra-strut neointima which appear to have been separated from the polymer intimately associated with the struts. These lacunae were interpreted to represent the deposition/migration of the strut-associated polymer/drug into the surrounding neointima. Since lacunae were not observed in the bare metal stents similarly implanted, their presence in the coated stent tissue samples may be the local effects of neointimal formation inhibition secondary to the presence of the polymer (i.e., space-occupying mass) and/or sirolimus (i.e., smooth muscle cell inhibition).

Neointimal lacunae were only observed only at Day 30 (when evaluated at about days 3, 30, 90, 180, and 365), whether there was a single coated stent implanted, or whether there were two coated stents implanted (overlapping as noted elsewhere herein). Thus, the polymer was resorbed (or absorbed) by day 90, at the latest. In some embodiments the polymer is resorbed (or absorbed) between day 30 and day 90. The magnitude of extra-strut neointimal lacunae (i.e., polymer/drug) was minimal, and though they were commonly seen on a per plane basis (˜70%), within each affected plane the change was generally limited to only one to two foci. Regardless, the presence of neointimal lacunae after 30 days implantation of the coated stents implanted as noted herein did not appear to be associated with any adverse tissue response. Rarely (˜<5%), lacunae were present in the adventitia, with associated inflammation, and usually the result of mural injury.

Full absorption (by the arterial tissue, but the vessel, or by the body, for example) exists when at least 75%, at least 80%, at least 90%, at least 95%, at least 99%, or 100% of the polymer is not visible in histology tissue sections tested by histological evaluation methods of U.S. application Ser. No. 13/445,723, and visualized according to methods also in U.S. application Ser. No. 13/445,723, at least, or by other evaluation methods known to one of skill in the art. In some embodiments, this may mean that an evaluation of artifacts of the polymer is evaluated, rather than finding polymer itself in the sample. Artifacts may include the clear spaces in the histological samples attributable to polymer (and not the stent struts).

In some embodiments, a bare metal stent is achieved by 45 days at most, as demonstrated by clearance of the coating from the stent, such as by measuring the amount of drug on the stent. In some embodiments, a bare metal stent is achieved by 45-60 days at most, as demonstrated by clearance of the coating from the stent, such as by measuring the amount of drug on the stent. In some embodiments, a bare metal stent is achieved by 90 days at most, as demonstrated by clearance of the coating from the stent, such as by measuring the amount of drug on the stent. In some embodiments, a bare metal stent is achieved within 45-90 days, as demonstrated by clearance of the coating from the stent, such as by measuring the amount of drug on the stent. In some embodiments, a bare metal stent is achieved within 45-60 days, as demonstrated by clearance of the coating from the stent, such as by measuring the amount of drug on the stent. Clearance of the coating from the stent may be when over 52% of the sirolimus is no longer associated with the stent thus over 52% of the coating is released from the stent, at least 75% of the sirolimus is no longer associated with the stent thus at least 75% of the coating is released from the stent, at least 80% of the sirolimus is no longer associated with the stent thus at least 80% of the coating is released from the stent, at least 90% of the sirolimus is no longer associated with the stent thus at least 90% of the coating is released from the stent, at least 95% of the sirolimus is no longer associated with the stent thus at least 95% of the coating is released from the stent, at least 97% of the sirolimus is no longer associated with the stent thus at least 97% of the coating is released from the stent as measured according to detection methods described herein (e.g. by measuring the amount of drug on the stent) or known to one of skill in the art.

In this example a thin strut (64 μm) Co Cr GENIUS® Magic coronary Stent & Rx Catheter was used. The stent was coated according to RESS methods described herein using a PDPDP sequence of steps to produce the coated stent. In this example, the PDPDP sequence of steps comprises Polymer single spray, sinter, Drug spray, Polymer double spray, sinter, Drug spray, Polymer triple spray, sinter. In some embodiments, the PDPDP sequence of steps comprises a first Polymer spray, sinter, Drug spray, a second Polymer spray that is about twice as long as the first Polymer spray, sinter, Drug spray, third Polymer spray that is about three times as long as the first Polymer spray, sinter. In some embodiments, the PDPDP sequence of steps comprises a first Polymer spray, sinter, Drug spray, a second Polymer spray that deposits about twice as much Polymer as the first Polymer spray, sinter, Drug spray, third Polymer spray deposits about three times as much Polymer as the first Polymer spray, sinter.

The Polymer was PLGA 50:50 having a number average molecular weight of about 15 kD. The drug was sirolimus in crystalline form (or at least partially crystalline). The resulting coated stent comprised crystalline sirolimus having a controlled elution profile similar to or equivalent to the profile as shown and described with respect to FIG. 2. The nominal target drug loading on the stents used was according to Table 2. The actual drug loading on a stent, depending on the embodiment for a particular device, may be at least one of: the target+/−5%, the target+/−10%, the target+/−15%, the target+/−20%, the target+/−25%, the target+/−30%, the target+/−35%, the target+/−40%, the target+/−45%, the target+/−50%, at least 50% of the target, at least 75% of the target, at least 80% of the target, at least 85% of the target, at least 90% of the target, at least 95% of the target, at most 105% of the target, at most 110% of the target, at most 115% of the target, at most 120% of the target, at most 125% of the target, at most 130% of the target, at most 140% of the target, and at most 150% of the target.

TABLE 2
Nominal Drug Target loading (in micrograms Sirolimus)

| Target | 9 mm | 15 mm | 19 mm | | 30 mm |
|---|---|---|---|---|---|
| Loading | length | length | length | 23 mm length | length |
| 7 cell stents | 83 μg | 135 μg | 175 μg | 214 μg | 280 μg |
| 9 cell stents | 107 μg | 165 μg | 210 μg | 253 μg | NA |

Enrollment in the trial was designed to be 30 patients at 5 sites—all implanted with the coated stent as described in this example. Efficacy was evaluated by reviewing in-stent late lumen loss (LLL) by QCA at each time point. There were exclusive groups of 10 patients at 4, 6 and 8 months follow-up. The Safety evaluation was conducted by reviewing MACE (death, MI and TVR). Mechanistic studies were performed using IVUS and OCT to understand the timeline for vessel healing at each time point. Additionally, angiographic evaluation was conducted at the follow-up time points. Furthermore, patients are to be followed for 5 years (data not available at the time of drafting).

The results were as follows. All 30 patients returned for 4, 6, and 8 months imaging follow up visits. Regarding the MACE Safety endpoint evaluation results are presented in Table 3. All events were adjudicated by CEC.

TABLE 3
Safety-MACE

| n = 30 | In-Hospital | <30 Days | <8 months |
|---|---|---|---|
| MACE | 1 | 1 | 2 |
| Death | 0 | 0 | 0 |
| Q wave MI | 0 | 0 | 0 |
| Non Q wave MI | 0 | 0 | 1** |
| Peri-procedural MI | 1* | — | — |
| TVR | 0 | 0 | 0 |
| Stent Thrombosis | 0 | 0 | 0 |

*due to elevation of cardiac enzymes only- with no elevation of CK, CK-MB 3.5 × ULN at 8-hours i.e. CK-MB elevation only post-procedure, 5.75 × ULN at 24 hours
**non-TL, non-Q wave MI: increased trooping post diagnostic angiogram at 44 days post-procedure

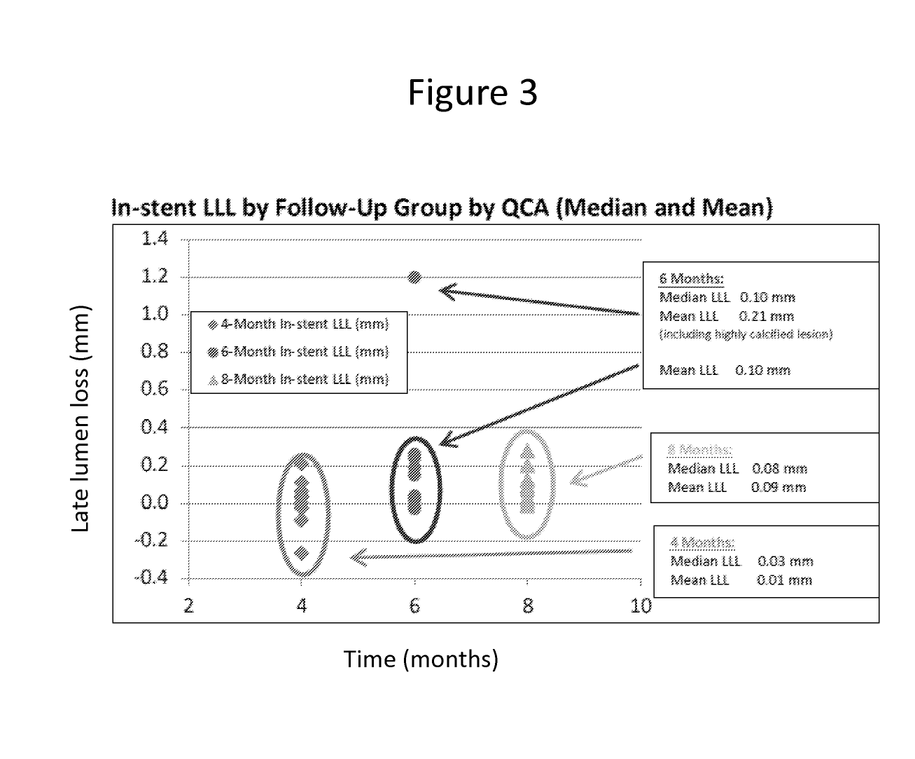

FIG. 3 shows the Patient level in-stent LLL by follow-up group, indicating no binary restenosis and having a linear regression indicating minimal change in LLL between 4 and 8 months. The x-axis is depicted in months, while the y-axis is the amount of in-stent LLL given in mm. The mean LLL at 4 months was determined to be 0.01 mm with a standard deviation of 0.12 mm; the median LLL at 4 months was determined to be 0.03 mm. The mean LLL at 6 months was determined to be 0.21 mm with a standard deviation of 0.36 mm; the median LLL was 0.10 mm. Note that there appeared to be a single statistical outlier in this 6 month group, which was attributed to the lesion of this patient being highly calcified and resulting in under-expansion of the stent, in contrast to the other lesions of the other patients. If this outlier is removed, the mean LLL at 6 months was 0.10 mm. The LLL at 8 months was determined to be 0.09 mm with a standard deviation of 0.10 mm; the median LLL was 0.08 mm. The regression line of the data (excluding the single outlier in the 6 month group) is y=0.019x−0.0098 (or −0.0098+0.0192*time). The QCA results as shown in FIG. 3 and in Table 4, at least, demonstrate a sustained and effectively suppressed neointimal hyperplasia. The results also show no binary restenosis, and linear regression of the data shown indicates minimal change in late lumen loss between 4 and 8 months.

Table 4 shows the Angiography results (QCA) for each of the 4 month, 6 month, and 8 month group. The median, range, and mean and standard deviation is provided for each group regarding in stent LLL. This is the same data that is presented graphically in FIG. 3.

TABLE 4
Angiography Results (QCA)

| | 4-month group | 6-month group | 8-month group |
|---|---|---|---|
| In-stent LLL (mm) | (n = 10) | (n = 10) | (n = 10) |
| Median | 0.03 | 0.10 | 0.08 |
| Range | −0.27 to 0.21 | −0.03 to 1.20 | −0.02 to 0.28 |
| Mean +/− SD | 0.01 +/− 0.12 | 0.21 +/− 0.36 | 0.09 +/− 0.10 |

In cross sectional analysis, vessel, stent and lumen borders were manually traced and neointimal area was obtained as stent area minus lumen area. To analyze the complete stented segment, volume parameters were generated using Simpson's method/rule: Volume equals the sum of multiple segments (S1 through Sn) i.e. Sum of Segment 1, Segment 2, Segment 3 . . . Segment n−1, Segment n. The volumes include vessel, lumen, stent, and neointima. To adjust for different stent lengths, the volume data were divided by stent length, and this volume index was shown as volume data. (Volume Index=Volume/length (mm³/mm)). To evaluate the overall magnitude of neointimal suppression of DES, the percent neointimal volume (% Neointimal volume Obstruction (% NIV)) was defined as neointimal volume divided by the volume and expressed as a percent. Cross-sectional narrowing percentage was defined as neointimal area divided by stent area (×100, i.e. expressed as a percent) to assess the most severe impact of neointima on luminal encroachment. To assess gross coverage of struts, neointima-free frame ratio (the ratio of frames without neointima) was also calculated and expressed as a percent. In each group certain results were not interpretable, and only interpretable results are presented with sample sizes as indicated (e.g. 1 case at 4 months, 3 cases at 6 months, and 2 cases at 8 months were not interpretable).

Table 5 shows the IVUS (3-D) results for each of the 4 month, 6 month, and 8 month group with respect to neointimal obstruction (%), neointimal volume index (mm³/mm), and late area loss (mm²). The entire stent and adjacent reference segment up to 5 mm were analyzed by IVUS. IVUS results demonstrate low neointimal hyperplasia.

TABLE 5
IVUS results

| | 4-month group | 6-month group | 8-month group |
|---|---|---|---|
| | Mean (SD) | Mean (SD) | Mean (SD) |
| Parameter | (n = 9) | (n = 7) | (n = 8) |
| neointimal obstruction | 5.2 (+/−3.2) | 8.0 (+/−3.1) | 10.9 (+/−4.6) |
| (%) | | | |
| neointimal volume | 0.3 (+/−0.1) | 0.7 (+/−0.4) | 0.8 (+/−0.5) |
| index (mm³/mm) | | | |
| late area loss (mm²) | 0.4 (+/−0.6) | 0.7 (+/−0.8) | 0.8 (+/−0.8) |

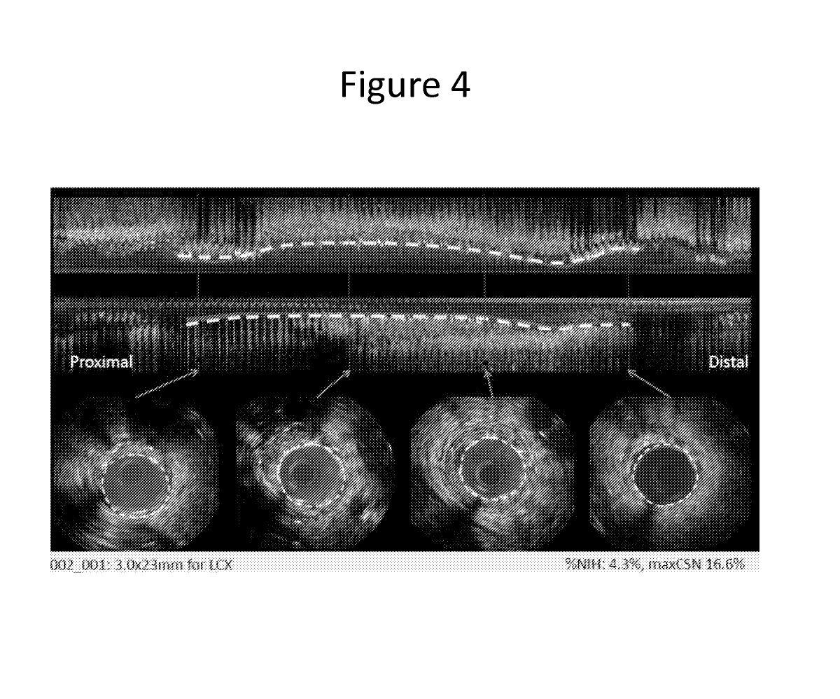

FIG. 4 shows a target artery and lesion of a single patient from the study in this example viewed by IVUS at 8 months follow up.

FIG. 5 shows a histogram of Neointimal obstruction of devices of this example at 4 months follow up as tested and analyzed using IVUS. The majority of the patient's neointimal obstruction was under 10%. The x-axis shows the percent neointimal obstruction, while the y-axis is sample size (N). The Average percent neointimal obstruction at 4 months was 5.2%+/−3.2%, while the median was 5.3%, the 25% Quartile was 3.0% and the 75% Quartile was 5.85.

The average percent of maximum cross sectional narrowing was detected and determined by IVUS at 4 months to be 11.9%+/−4.6% (n=9), and there was no case in which there was lumen encroachment (which would be an encroachment over 50%). The neointima free frame ratio at 4 months was determined to be 20%+/−17%. Late area Loss was 0.4+/−0.6 mm², and the minimum lumen area in the stented segment appeared to trend to decrease between baseline (at implantation) which had a minimum lumen area in the stented segment of 5.9+/−1.4 mm² to 4 month follow up which had a minimum lumen area in the stented segment determined to be 5.5+/−1.1 mm², but the decrease was not statistically significant (p=0.122).

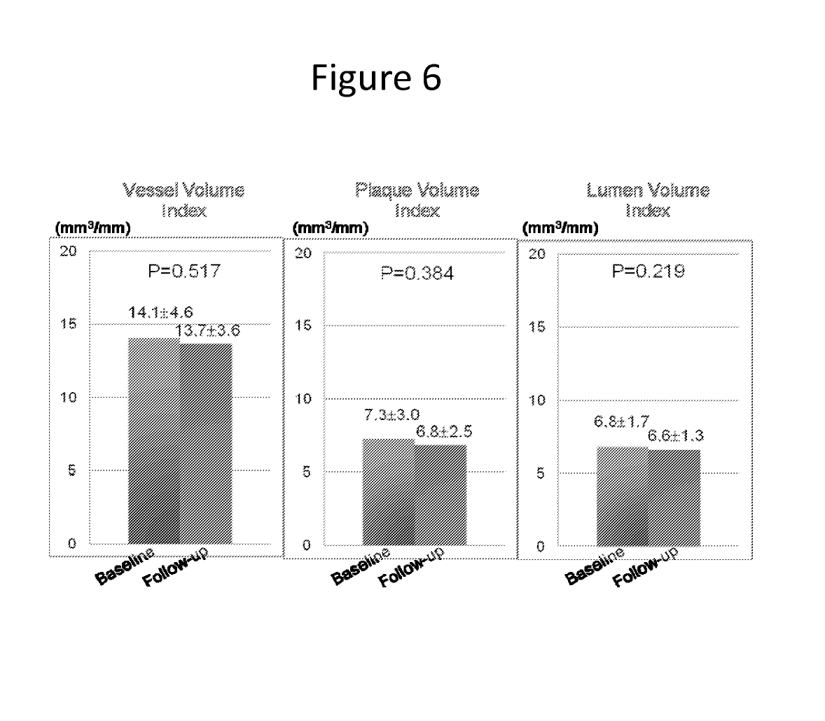

FIG. 6 shows Vessel Response in this study, which shows Vessel Volume Index, Plaque Volume Index, and Lumen Volume Index at baseline (at implantation) and at 4 months follow up. No significant changes were observed in any of these Indexes.

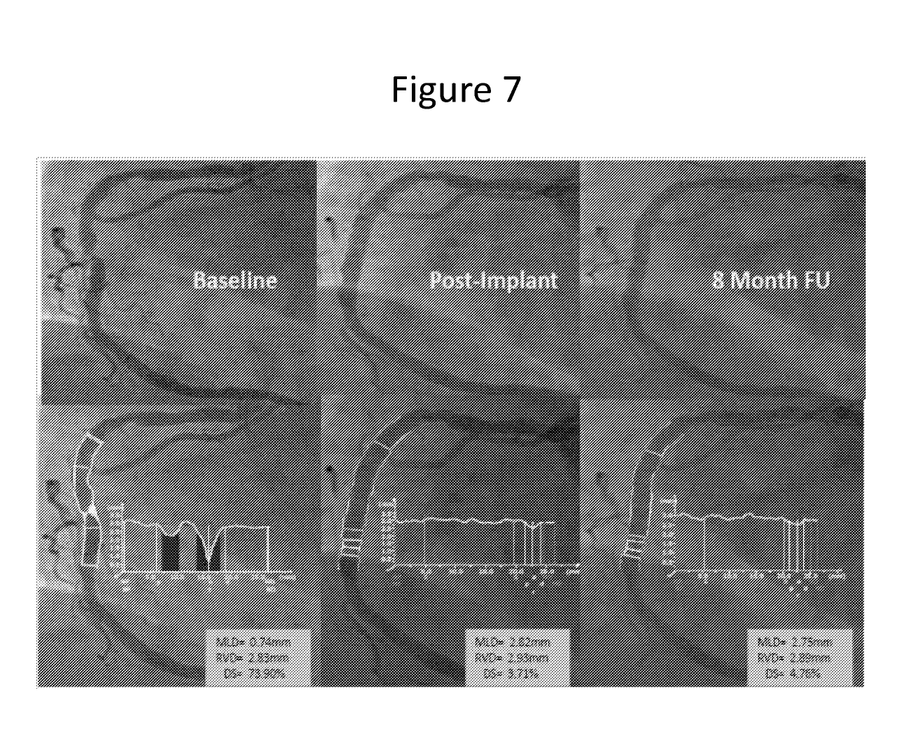

FIG. 7 shows the target artery and lesion of a single patient from the study in this example viewed under fluoroscopy prior to implantation of the device from this study (first images top and bottom labeled “Baseline”), just after implantation of the device (middle images top and bottom labeled “Post-Implant”) and at 8 months follow up (last top and bottom images labeled “8 Month FU”).

In the qualitative analysis using IVUS at 4 month follow up, both tissue prolapse in one case and stent edge dissection (proximal end) in another case was observed. In the serial IVUS analysis, both resolved incomplete stent apposition (edge) in one case and late acquired incomplete stent apposition (body) in another were observed. Table 6 shows the OCT results with respect to strut coverage (%) for each of the 4 month, 6 month, and 8 month groups. The OCT results show a high rate of stent strut coverage at all time points, indicating safety of the device.

TABLE 6
OCT results

| Strut Coverage (%) | 4-month group | 6-month group | 8-month group |
|---|---|---|---|
| Median | 90% | 97% | 96% |
| Mean | 85% | 93% | 96% |

Results of this example study indicate that the device as designed according to methods described herein and having the features noted herein results in a complete absorption of the polymer in 90 days leaving a bare metal stent. The device as shown and described herein has rapid, uniform neointimal coverage with no adverse vessel reaction at four months follow up, at least. The late lumen loss and percent (%) obstruction show good inhibition of neointimal hyperplasia. This is demonstrated at least by: in stent LLL at 8 months was 0.09 mm; the percent neointimal obstruction at 8 months was 10.9%; and/or there were no incidences of binary restenosis or revascularizations. In the 4 month follow up analysis, no significant changes were observed in vessel volume index, plaque volume index, or lumen volume index. Neointimal obstruction at 4 months was minimal, and no case showed significant lumen encroachment. Neointima-free frame ratio was 20.2+/−16.6%, indicating the large part of the stented segments covered with IVUS-detectable neointima even at 4 months follow up. Late acquired incomplete apposition was observed in one case. Furthermore, OCT demonstrates good strut coverage at all time points. Good strut coverage is demonstrated by OCT evaluation of strut coverage as noted herein and shows that at least 80% of the struts are covered on average at each of 4 months, 6 months and 8 months following implantation of the device. Good strut coverage is demonstrated by OCT evaluation of strut coverage as noted herein and shows that at least 80% of the struts are covered on average at 4 months, and at least 90% of the struts are covered on average at 6 months and at 8 months following implantation of the device. Good strut coverage is demonstrated by OCT evaluation of strut coverage as noted herein and shows that at least 80% of the struts are covered on average at 4 months, at least 90% of the struts are covered on average at 6 months, and at least 95% of the struts are covered on average at 8 months following implantation of the device. Good strut coverage is demonstrated by OCT evaluation of strut coverage as noted herein and shows that at least 85% of the struts are covered (median) at each of 4 months, 6 months and 8 months following implantation of the device. Good strut coverage is demonstrated by OCT evaluation of strut coverage as noted herein and shows that at least 85% of the struts are covered (median) at 4 months, and at least 95% of the struts are covered (median) at 6 months and at 8 months following implantation of the device.

The devices exhibit good efficacy and safety through 8 months follow up and improved safety profile as compared to current DES made by other methods such as using solvent based coating methods wherein the drug is amorphous in form. The devices as described herein and/or as described in this study provide controlled, continuous, sustained release of drug over 6 months, without an initial drug burst into the tissue surrounding the device or into the blood stream (as exists in current DES devices). Efficacy appears comparable or improved as compared to current DES devices. Absence of polymer coating in the tissue (after 90 d), or on the stent (once cleared from the stent at 45-60 days) may mitigate hypersensitivity, impaired healing, and abnormal vasomotor function. The devices as described herein can thus reduce risks of DAPT non-compliance and/or interruption. They can be used on high-risk patients. They can reduce or eliminate risks of permanent coating such as long term thrombosis risks. No stent thrombosis was detected in any patient in the study as tested through 8 months.

### Example 3

12-month data from an in-human clinical study of a sirolimus eluting absorbable polymer coronary stent system is presented in this example. The 4 month, 6 month, and 8 month data for the same study was presented in Example 2, at least, above. The sirolimus eluting absorbable polymer coronary stent system comprises a thin-strut drug-eluting stent distinguished by a rapid-absorbing drug/polymer coating designed for controlled drug release. The clinical trial assessed safety and efficacy and had an important mechanistic design component providing detailed data on vessel “healing” following polymer absorption. Thirty subjects were treated with a stent produced according to Example 1 or 2, at least, and then evaluated at 4, 6 and 8 months by angiography, intravascular ultrasound (IVUS), and optical coherence tomography (OCT).

There were no reports of target lesion or vessel revascularization, or stent thrombosis with the implanted stents. The major adverse cardiac events (MACE) rate at 12-months was 3.3%, reflective of a non Q-wave MI in a vessel treated with a commercial stent. The primary efficacy endpoint was in-stent late lumen loss (LLL). The median in-stent late lumen loss was low at 0.03, 0.10 and 0.08 mm at 4, 6 and 8 months, respectively. The median percent of stent struts covered with tissue was 90%, 97% and 96% at 4, 6 and 8 months, respectively, with no positive remodeling. Angiography, intravascular ultrasound (IVUS) and optical coherence tomography (OCT) imaging results were measured by independent core laboratories.

Durable polymers on coated stents currently in use have been linked with very late stent thrombosis, which can surface at more than one year post-procedure. Contributing factors can include one or more of: chronic inflammation stimulus, incomplete re-endothelialization, acquired incomplete stent apposition, positive remodeling, and local allergic reactions. Absorbable polymers, on the other hand, if properly used (such as used herein) can provide the same safety profile as bare metal stents, can decrease late stent thrombosis events, and/or can shorten the time requirements for DAPT, which may allow patients to suffer less from non-compliance, or undergo additional surgical procedures sooner after the intervention. The stents produced as noted herein increase patient safety while achieving equivalent or better efficacy than currently is available with existing products. The absorbable polymer used in the coated stents implanted in this example (designed as noted in Example 1, at least) may decrease stent thrombosis events, provide the desirable safety profile of bare metal stents and shorten the duration of dual antiplatelet therapy.

The human implantation of the coated stent in this Example represents the first clinical assessment of safety and efficacy of the sirolimus eluting absorbable polymer stent system, treated thirty patients (subjects) with de novo lesions in coronary arteries ranging in diameter from 2.5 to 3.5 mm and amenable to treatment with a maximum 23 mm length stent (19 or 23 mm stent lengths used). In order to be part of the study, the subject needed to have stable or unstable angina pectoris (Class I, II, III or IV), documented ischemia, or documented silent ischemia. The subject was not in the study if they had recent Q wave MI (<72 hrs) or no elevated cardiac biomarkers. Subjects were excluded if the target lesion was highly calcified, tortuous, thrombus present, proximal angulation, and excluded if the target lesion was located at side branch >2.5 mm, ostial location, previously treated vessel. It was acceptable to treat one critical non-target lesion in another vessel prior to target lesion. Subjects were enrolled across five study centers in New Zealand, Australia and Belgium. Three independent subgroups of 10 patients each were evaluated using angiography, IVUS and OCT at three time points: four, six and eight months. All 30 patients were evaluated for major adverse cardiac events at 12 months. The primary efficacy endpoint was in-stent late lumen loss. Safety was assessed by incidence of major adverse cardiac events (death, Q and non Q wave MI or target vessel revascularization) and presence of strut coverage with tissue within the treated artery at each time point. Patients were to be followed at 18 months (angiography, IVUS, and OCT) and annually for 4 years.

In accordance with ACC/AHA/SCAI PCI Practice Guidelines, the DAPT recommendations were for Aspirin indefinitely and Plavix® (clopidogrel) for 12 mo, with (minimum of 6 months taking local country recommendations into consideration). Pre-procedure, 87% of patients were already on DAPT. Patient compliance to DAPT was at 6 months 83%, at 9 months 80%, and at 12 months 60% compliance.

The demographics, risk factors, and lesion characteristics were as described in Example 2, Table 1. The procedure included mean % DS pre-procedure (mean %+/−SD) 69.3+/−7.7; maximum stent deployment pressure (in mean atm+/−SD) of 13.9+/−2.7; and number of non-TV lesions treated were (as a percent) 26.7% (i.e. 8/30). Device success was determined existing in 96.6% ( 29/30) of the cases. A single device was unable to cross the lesion due to calcification in the artery, a subsequent device was implanted. Lesion success was 100%, and procedural success was 100%. Angiographically speaking (expressed as mean+/−SD): the in-stent % DS following the procedure was 2.26% (±7.44); the in-lesion % DS following the procedure was 13.37% (±7.39); the in-stent MLD (in mm) following the procedure was 2.83 mm (±0.30); and the the in-lesion MLD (in mm) following the procedure was 2.51 mm (±0.28).

The sirolimus eluting absorbable polymer coronary stent system is designed to optimize healing in patients with coronary artery disease. The rapid-absorbing drug/polymer coating is intended to precisely and consistently control drug elution and polymer exposure duration to reduce the safety risks associated with current commercially available drug eluting stent technologies.

The sirolimus eluting absorbable polymer coronary stent system includes a stent coating that contains crystalline drug (sirolimus) and an absorbable polymer. As the polymer softens and disperses from the stent into the adjacent tissue, the coating provides controlled and sustained release of therapeutic levels of drug within the surrounding tissue. Results of animal studies have determined that the drug/polymer coating is cleared from the stent in 45 to 60 days leaving a bare metal stent and the polymer is completely absorbed into the surrounding tissue in 90 days to promote long-term patency and compatibility with the artery.

Using an approved drug (sirolimus) and polymer (PLGA), the supercritical fluid technology allows a rigorously controlled drug/polymer coating to be applied to a bare-metal stent. The sirolimus eluting absorbable polymer coronary stent system leverages the benefits of Eurocor's Genius® MAGIC cobalt chromium coronary stent system, a state-of-the-art bare-metal stent, which has demonstrated excellent deliverability, conformability and flexibility.

Using the methods herein, as demonstrated in this and other examples, at least, it is possible to precisely and consistently control drug elution and polymer exposure duration, creating the potential for a therapeutic solution to coronary artery disease without the long-term safety concerns of currently available drug-eluting stents.

Polymer generally has been linked to DES (drug eluting stent) complications, including delayed vessel healing, hypersensitivity reaction, stent thrombosis (ST), acute myocardial infarction (MI) and sudden death. Strut thickness generates flow disruption and exacerbate thrombogenicity. An absorbable polymer (absorption in 90-days) and a thin stent (64 μm-thick cobalt chromium, for example) is used and evaluated in this example, at least; the exact aspects of which are described in this example, at least. Using this device, DES safety can be increased, while maintaining efficacy, by means of this thin stent platform coupled with optimized drug delivery and reduced vessel exposure to polymer.

Intravascular frequency-domain optical coherence tomography (FD-OCT) is a high-resolution (10 μm) imaging modality, which enables detailed analysis of stent-vessel interaction. The patients in each group (4 months—10 patients, 6 months—10 patients with one excluded, and 8 months—10 patients with one excluded) completed FD-OCT follow-up.

Every single strut in the cross section evaluated was analyzed for distance from its luminal surface to the lumen contour, definition of malapposed struts was provided during the post-processing phase based on the knowledge of the total strut thickness (strut+polymer+drug), and a qualitative call was used for protruding and LIT struts.

A coefficient of variation (CV) was determined to establish a distribution of uncovered struts (stent-level). The CV=the standard deviation of % uncovered struts per 2.5 mm stent subsegment divided by the mean of % uncovered struts per 2.5 mm stent subsegment. For example, in one case the percent of total uncovered struts was 2.8%, but the CV was 120.6. In another case, the percent of total uncovered struts was also 2.8%, but the CV was 80.6. The CV of the former case shows homogeneous distribution of uncoverage by 2.5 mm subsegment assessment, as compared to the latter case.

Tissue Characterization based on OD was also determined. Optical Density (Pixel Intensity or “PI”) of the tissue covering the stent struts normalized for the optical density of the stent struts. Optical Density (OD)=PI of tissue/PI of strut blooming. As an example of how this can be helpful, in one case a pig model was implanted with a device and at three days the OD was determined to be 0.57, demonstrated by the tissue mean intensity of 4.12 divided by the stent mean intensity of 7.15. In another case a pig model was implanted with a device herein and at 180 days the OD was determined to be 0.80, demonstrated by the tissue mean intensity of 5.78 divided by the stent mean intensity of 7.22. At three days, the OD indicated fibrin, whereas at 180 days, the OD indicated neointimal hyperplasia (NIH).

Statistical analysis was done of the data collected in this example. The continuous variables are presented as median and the difference among three time points was analyzed by Kruskal-Wallis test with post-hoc Mann-Whitney U tests with the Bonferroni correction. Based on the results noted in Table 7, significant difference was detected between 4 M and 8 M with Mann-Whitney U test, p<0.05/3 after Bonferroni correction.

TABLE 7

| Planar | | | | | p Value 4 | P Value 4 | Planar |
|---|---|---|---|---|---|---|---|
| analysis | 4 M | 6 M | 8 M | P* Value | vs. 6 M | vs. 8 M | analysis |
| Stent | 6.22 | 7.19 | 6.82 | 0.415 | 0.236 | 0.348 | Stent |
| area, mm² | (5.08, 7.84) | (5.57, 9.84) | (6.03, 8.95) | | | | area, mm² |
| Lumen | 6.20 | 6.36 | 6.30 | 0.643 | 0.348 | 0.653 | Lumen |
| area, mm² | (4.67, 7.29) | (5.73, 8.88) | (5.95, 7.63) | | | | area, mm² |
| Minimal | 5.23 | 5.52 | 5.02 | 0.934 | 0.713 | 0.967 | Minimal |
| lumen | (4.04, 6.03) | (2.80, 7.90) | (4.48, 5.89) | | | | lumen |
| area, mm² | | | | | | | area, mm² |
| Neointimal | 0.47 | 0.85 | 1.19 | 0.012* | 0.025 | 0.010 | Neointimal |
| area, mm² | (0.19, 0.54) | (0.72, 0.99) | (0.58, 1.33) | | | | area, mm² |
| Malapposit. | 0.03 | 0.01 | 0.04 | 0.844 | 0.540 | 0.775 | Malapposit. |
| area, mm² | (0.01, 0.11) | (0.00, 0.32) | (0.00, 0.07) | | | | area, mm² |
| Stenosis | 6.89 | 10.05 | 13.73 | 0.035 | 0.111 | 0.020 | Stenosis |
| (%) | (3.06, 8.94) | (8.10, 11.86) | (10.12, 17.90) | | | | (%) |

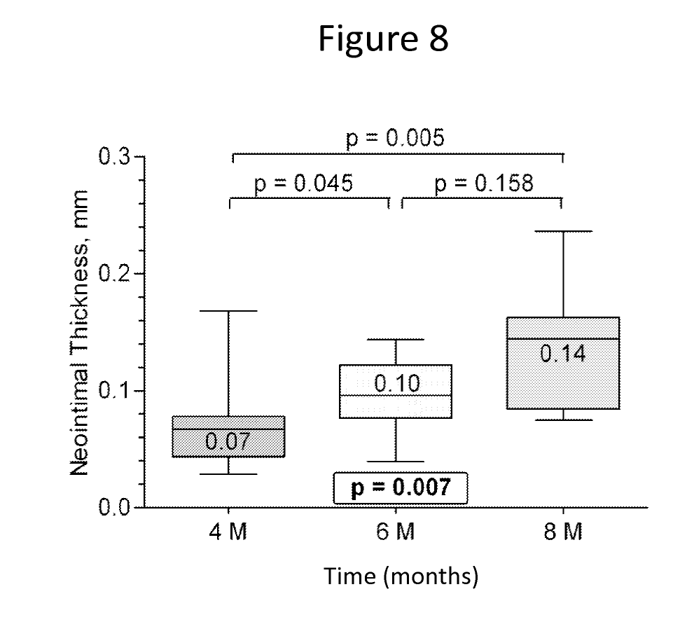

FIG. 8 depicts the results of neointimal thickness in mm, including statistical results comparing the groups as noted, as described in this example, at least. It shows that the mean neointimal thickness at 4 months was 0.07 mm, at 6 months was 0.10 mm, and at 8 months was 0.14 mm, with a p value between 4 months and 6 months of 0.045, between 4 months and 8 months was 0.005, between 6 months and 8 months was 0.158, and among all groups (4 mo, 6 mo, 8 mo) was 0.007.

Table 8 shows the neointimal hyperplasia (NIH) distribution (cross section level) of the subjects in this study, expressed as a neointima exocentricity index (NEI). It can be said that these results, at 6 months follow-up, are in alignment (not statistitcally significantly different) than other studied devices such as Resolute Long OCT, Everest (CoCr) Xience V, Everest (PtCr) Xience V.

TABLE 8

| | | | | | p Value 4 | p Value 4 | p Value 6 |
|---|---|---|---|---|---|---|---|
| | 4 M | 6 M | 8 M | p Value | vs. 6 | vs. 8 | vs. 8 |
| Neointima | 3.07 (2.60 | 2.47 (2.26 | 2.43 (2.05 | 0.200 | 0.206 | 0.111 | 0.596 |
| Eccentricity | min, 4.61 | min, 3.56 | min, 2.85 | | | | |
| Index (NEI) | max) | max) | max) | | | | |

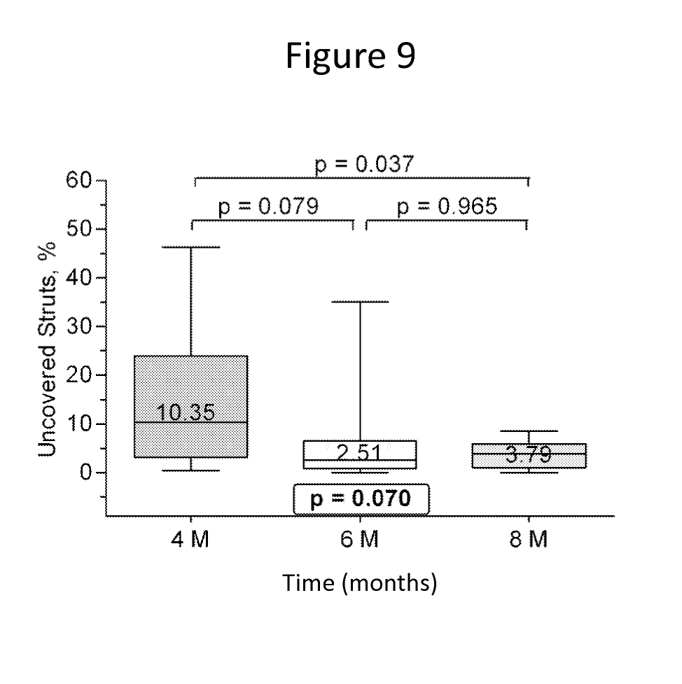

FIG. 9 depicts the uncovered struts percent of the 28 patients of the study, including an evaluation of 7403 stent struts total, and includes statistical results comparing the groups noted, as described in this example, at least. It shows that the mean uncovered struts percent at 4 months was 10.35%, at 6 months was 2.51%, and at 8 months was 3.79%, with a p value between 4 months and 6 months of 0.079, between 4 months and 8 months was 0.037, between 6 months and 8 months was 0.965, and among all groups (4 mo, 6 mo, 8 mo) was 0.070.

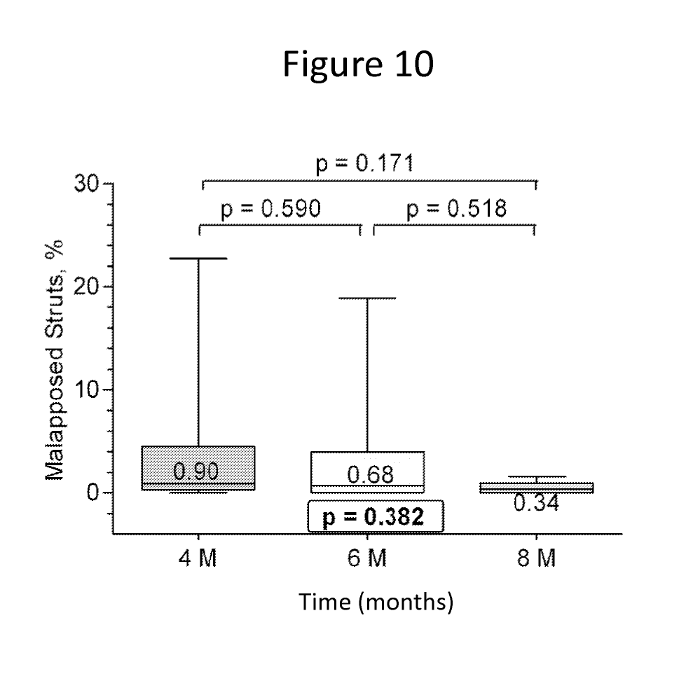

FIG. 10 depicts the malapposed struts percent of the 28 patients of the study, and includes statistical results comparing the groups noted, as described in this example, at least. It shows that the mean malapposed struts percent at 4 months was 0.90%, at 6 months was 0.68%, and at 8 months was 0.34%, with a p value between 4 months and 6 months of 0.590, between 4 months and 8 months was 0.171, between 6 months and 8 months was 0.518, and among all groups (4 mo, 6 mo, 8 mo) was 0.382.

Table 9 shows the additional results of the study, including number of patients with >30% uncovered struts, maximum length of the uncovered segment, maximum length of any malapposed segment, any patient with AIT related to uncovered strut (none at any time point), and any patient with AIT related to malapposed struts (none at any time point).

TABLE 9

| | | | | | p Value 4 | p Value 4 | p Value 6 |
|---|---|---|---|---|---|---|---|
| | 4 M | 6 M | 8 M | p Value | vs. 6 | vs. 8 | vs. 8 |
| Patients | 2 (20.0) | 1 (11.1) | 0 (0.0) | 0.753 | 1.000 | 0.474 | 1.000 |
| with >30% | | | | | | | |
| uncovered | | | | | | | |
| struts, n (%) | | | | | | | |
| Maximum | 2.70 (1.80 | 1.20 (0.60 | 2.00 (0.60 | 0.164 | 0.064 | 0.268 | 0.563 |
| length of | min, 6.20 | min, 1.80 | min, 3.40 | | | | |
| uncovered | max) | max) | max) | | | | |
| segment, mm | | | | | | | |
| Maximum | 0.60 | 0.60 | 0.60 | 0.189 | 0.332 | 0.072 | 0.504 |
| length of | (0.60, | (0.00, | (0.00, | | | | |
| malapposed | 2.00) | 1.20) | 0.60) | | | | |
| segment, mm | | | | | | | |
| Patient with | 0 (0.0) | 0 (0.0) | 0 (0.0) | NA | NA | NA | NA |
| AIT related | | | | | | | |
| with uncovered | | | | | | | |
| strut, n (%) | | | | | | | |
| Patient with | 0 (0.0) | 0 (0.0) | 0 (0.0) | NA | NA | NA | NA |
| AIT related | | | | | | | |
| with malapposed | | | | | | | |
| struts, n (%) | | | | | | | |

Table 10 shows the CV of uncovered struts in 2.5 mm subsegments, as a %, and CV of malapposed struts in 2.5 mm subsegments, as a percent. It can be said that these results, at 6 months follow-up, are in alignment (not statistically significantly different) than other studied devices such as Resolute Long OCT, Everest (CoCr) Xience V, Everest (PtCr) Xience V.

TABLE 10

| | | | | | p Value 4 | p Value 4 | p Value 6 |
|---|---|---|---|---|---|---|---|
| | 4 M | 6 M | 8 M | p Value | vs. 6 | vs. 8 | vs. 8 |
| CV of uncovered | 99.88 (75.54 | 109.35 (92.13 | 156.64 (138.40 | 0.115 | 0.488 | 0.045 | 0.216 |
| struts in 2.5-mm | min, 141.03 | min, 158.02 | min, 242.17 | | | | |
| subsegment, % | max) | max) | max) | | | | |
| CV of malapposed | 208.59 (38.06 | 58.76 (0.00 | 182.77 (0.00 | 0.596 | 0.339 | 0.771 | 0.548 |
| struts in 2.5-mm | min, 244.95 | min, 129.50 | min, 244.95 | | | | |
| subsegment, % | max) | max) | max) | | | | |

With regard to tissue characterization, in evaluating normalized optical density and the percent of struts covered by fibrin (n/total), a significant difference was detected between 4 M and 8 M with Mann-Whitney U test, p<0.05/3 after Bonferroni correction. The threshold of normalized optical density utilized (0.610) to determine fibrin coverage (95% specificity) was based on pre-clinical longitudinal FD-OCT coupled with histological assessments. Table 11 shows the results of this tissue characterization.

TABLE 11

| | | | | | p Value 4 | p Value 4 | p Value 6 |
|---|---|---|---|---|---|---|---|
| | 4 M | 6 M | 8 M | p Value | vs. 6 | vs. 8 | vs. 8 |
| Normalized | 0.67 (0.63 | 0.73 (0.67 | 0.77 (0.76 | 0.002 | 0.054 | 0.002 | 0.070 |
| Optical Density | min, 0.69 | min, 0.76 | min, 0.78 | | | | |
| | max) | max) | max) | | | | |
| Struts Covered | 8.8 | 3.1 | 0.3 | 0.015 | 0.072 | 0.008 | 0.158 |
| by Fibrin, % | (50/570) | (9/286) | (1/353) | | | | |
| (n/total) † | | | | | | | |

The comprehensive assessment of vascular response performed at 4-, 6-, and 8-month follow-up after sirolimus eluting absorbable polymer coronary stent system implantation in human coronary arteries by means of FD-OCT revealed—Low rates of uncovered and malapposed struts, with a trend towards reduction in uncovered struts after 4 months; homogeneous distribution of uncovered and malapposed struts along the stent, and lack of abnormal intraluminal tissue related with uncovered or malapposed struts was identified.

Generally, durable polymers have been linked with very late (>1 yr) stent thrombosis events, evidence of which can be found in delayed endothelial coverage, impaired endothelial function, elevated luminal inflammatory cells, and increased fibrin deposition compared to bare metal stent controls. The sirolimus eluting absorbable polymer stent system evaluated in this Example (at least) eliminates risks associated with durable polymers while optimizing properties of absorbable drug eluting coatings. The devices herein are characterized by the following (at least): rapid elimination of the polymer from the stent (i.e. cleared from the stent) in 45-60 days; thin struts are used (˜64 microns on average) for enhanced deliverability which may reduce risk of acute thrombogenicity; effective known drug, sirolimus, known to be a powerful neointimal suppressor; and is safe, in that it provides controlled release of the drug from the absorbable polymer.

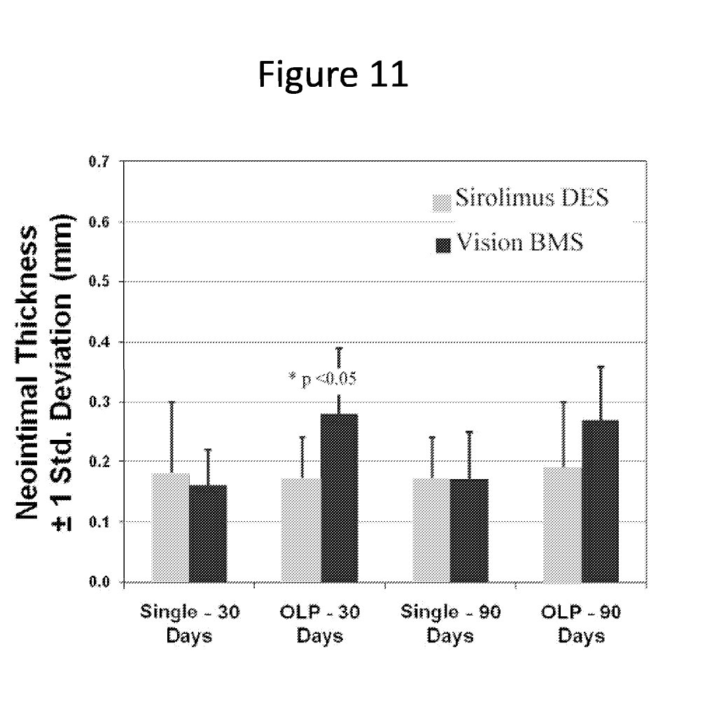

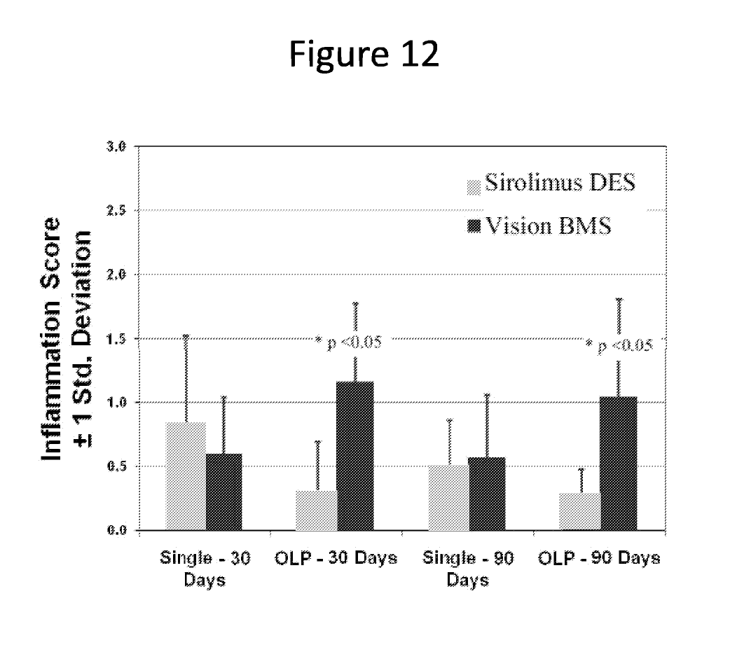

The device has been shown in a porcine model to result in significantly lower inflammation than BMS through 90 days in a challenging overlap configuration, and significantly less neointimal hyperplasia (NIH) through 30 days (see e.g., FIGS. 11 and 12). FIG. 11 shows the neointimal thickness score and standard deviation recorded at each of 30 days and 90 days in both a single and overlapping (OLP) Sirolimus DES (left column at each time point and condition) and Vision BMS (right column at each time point and condition) stent implantation in a porcine model. FIG. 12 shows the average inflammation score and standard deviation recorded at each of 30 days and 90 days in both a single and overlapping (OLP) Sirolimus DES (left column at each time point and condition) and Vision BMS (right column at each time point and condition) stent implantation in a porcine model.

The safety at 12 months was evaluated in terms of MACE (major adverse cardiac events), at least, defined as death, MI i.e. myocardial infarction (Q-wave myocardial infarction, or “QWMI” and non-Q-wave myocardial infarction or “NQWMI”) and TVR (target vessel revascularization). At 12 months, 100% of the patients were evaluated for MACE. Table 12 shows the Events (as adjudicated by CEC) at 12 months, showing safety of the device. In summary, the MACE rate was 3.35 with no TLR (target lesion revascularization), TVR (target vessel revascularization) or ST (stent thrombosis) at 12 months. This data for MACE was not changed from that found at 8 months (See Example 2, Table 3).

TABLE 12

| Events | Devices of Example 3 |
|---|---|
| as adjudicated by CEC | (N = 30 pts) |
| Total MACE* | 3.3% (1)* |
| Death | 0% |
| Q and non Q wave MI | 3.3% (1) |
| TVF | 0% |
| Peri-procedural MI | 6.7% (2)** |
| Stent Thrombosis | 0% |

*non TV, NQWMI: increased troponin following diagnostic angiogram at 44 days post-index procedure
**ppmi only: slight CK-MB elevation post-procedure, do not meet definition of non Q-wave MI

Angiographically speaking the primary endpoint of In-stent late lumen loss is shown in Table 13 (also expressed in Table 4 in Example 2).

TABLE 13

| In-Stent LLL | 4-Month Group | 6-Month Group | 8-Month Group |
|---|---|---|---|
| (mm) | (n = 10) | (n = 10) | (n = 10) |
| Mean (±SD) | 0.01 (±0.12) | 0.21* (±0.36) | 0.09 (±0.10) |
| Median | 0.03 | 0.10 (−0.03 to 1.20) | 0.08 |
| (Range) | (−0.22 to 0.21) | | (−0.01 to 0.28) |

*Includes outlier with lesion outside proximal end to stent that grew into the stent. Excluding outlier the mean 6-mo LLL was 0.1 mm

Patient Level in-stent LLL (QCA) by follow-up group was as depicted in FIG. 3, and as described in Example 2, at least. Neointimal Hyperplasia at 4 months, 6 months and 8 months were presented in Table 5 as noted and described in Example 2. Based on these IVUS results, there was no significant positive remodeling shown (defined as vessel volume index increase of >/=20%). Additionally, there was one case of late acquired incomplete stent apposition (ISA) at 6 months observed. OCT results showed a high rate of stent strut coverage at all time points (also depicted in Table 6 of Example 2, at least). Efficacy at 8 months was shown by the determination of in stent LLL of 0.09 mm, essentially no change in NIH from 6 months to 8 months follow up, and since there was no binary restosis or revascularizations at 8 months. These results, at least are also expressed in Example 2. Safety was also determined at 12 months, showing no MACE events (only one non-TV, NQWMI), good stent coverage of 96.5% at 8 months by OCT evaluation, and no stent thrombosis or revascularizations.

### Example 4

18-month data from an in-human clinical study of a sirolimus eluting absorbable polymer coronary stent system is presented in this example. The 4 month, 6 month and 8 month data for the same study was presented in Example 2 and the 12 month data for the same study was presented in Example 3, at least, above. The sirolimus eluting absorbable polymer coronary stent system comprises a thin-strut drug-eluting stent distinguished by a rapid-absorbing drug/polymer coating designed for controlled drug release. The clinical trial assessed safety and efficacy and had an important mechanistic design component providing detailed data on vessel “healing” following polymer absorption. Thirty subjects were treated with a stent produced according to Examples 1, 2 and 3, at least, and then evaluated at 4, 6, 8 and 18 months by angiography, intravascular ultrasound (IVUS), and optical coherence tomography (OCT).

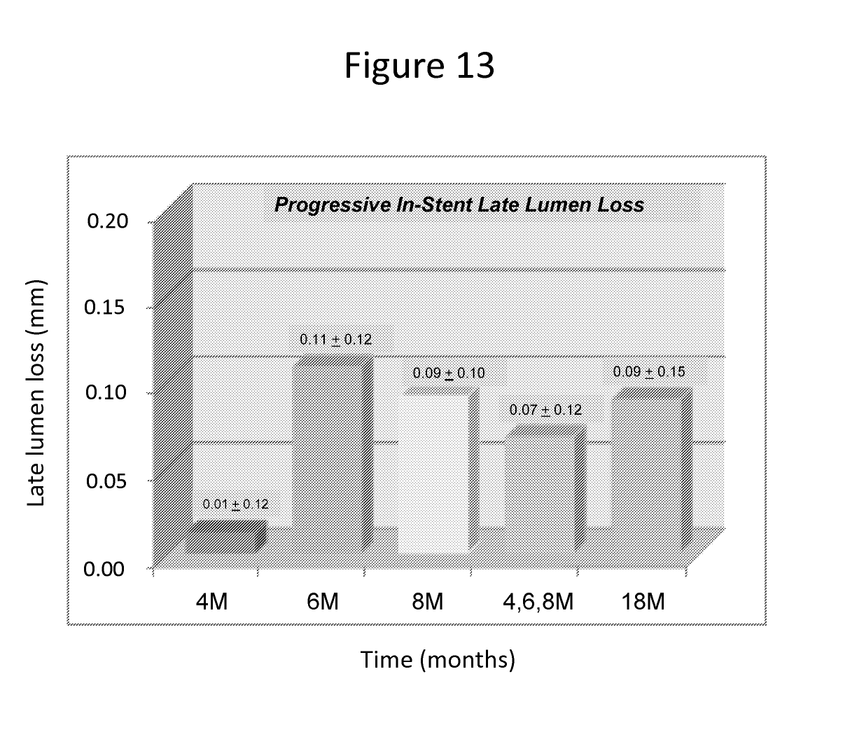

There were no reports of target lesion or vessel revascularization, or stent thrombosis with the implanted stents. The major adverse cardiac events (MACE) rate at 18-months was 3.3%, reflective of a non Q-wave MI in a vessel treated with a commercial stent. The primary efficacy endpoint was in-stent late lumen loss (LLL). The mean in-stent late lumen loss was low at 0.01, 0.11, 0.09, and 0.09 mm at 4, 6, 8 and 18 months, respectively (FIG. 13). The corresponding median in-stent late lumen loss was 0.03, 0.10, 0.08, and 0.08 mm at 4, 6, 8 and 18 months, respectively. Imaging with optical coherence tomography (OCT) demonstrated thin, homogeneous coverage with high rates of stent strut coverage and a low rate of stent strut malapposition. The mean percent of stent struts covered with tissue was 86%, 93%, 96%, and 99% at 4, 6, 8 and 18 months, respectively and the mean percent of uncovered malapposed struts was 4%, 3%, 0.5%, and 0.1% at 4, 6, 8 and 18 months, respectively. Angiography, intravascular ultrasound (IVUS) and optical coherence tomography (OCT) imaging results were measured by independent core laboratories.

The demographics, risk factors, and lesion characteristics were as described in Example 2, Table 1. The procedure included mean % DS pre-procedure (mean %+/−SD) 69.3+/−7.7; maximum stent deployment pressure (in mean atm+/−SD) of 13.9+/−2.7; and number of non-TV lesions treated were (as a percent) 26.7% (i.e. 8/30). Device success was determined existing in 96.6% ( 29/30) of the cases. A single device was unable to cross the lesion due to calcification in the artery, a subsequent device was implanted. Lesion success was 100%, and procedural success was 100%. Angiographically speaking (expressed as mean+/−SD): the in-stent % DS following the procedure was 2.26% (±7.44); the in-lesion % DS following the procedure was 13.37% (±7.39); the in-stent MLD (in mm) following the procedure was 2.83 mm (±0.30); and the in-lesion MLD (in mm) following the procedure was 2.51 mm (±0.28).

The sirolimus eluting absorbable polymer coronary stent system includes a stent coating that contains crystalline drug (sirolimus) and an absorbable polymer. As the polymer softens and disperses from the stent into the adjacent tissue, the coating provides controlled and sustained release of therapeutic levels of drug within the surrounding tissue. Results of animal studies have determined that the drug/polymer coating is cleared from the stent in 45 to 60 days leaving a bare metal stent and the polymer is completely absorbed into the surrounding tissue in 90 days to promote long-term patency and compatibility with the artery.

Using an approved drug (sirolimus) and polymer (PLGA), the supercritical fluid technology allows a rigorously controlled drug/polymer coating to be applied to a bare-metal stent. The sirolimus eluting absorbable polymer coronary stent system leverages the benefits of Eurocor's Genius® MAGIC cobalt chromium coronary stent system, a state-of-the-art bare-metal stent, which has demonstrated excellent deliverability, conformability and flexibility.

Using the methods herein, as demonstrated in this and other examples, at least, it is possible to precisely and consistently control drug elution and polymer exposure duration, creating the potential for a therapeutic solution to coronary artery disease without the long-term safety concerns of currently available drug-eluting stents.

Intravascular frequency-domain optical coherence tomography (FD-OCT) is a high-resolution (10 μm) imaging modality, which enables detailed analysis of stent-vessel interaction. The patients in each group (4 months—10 patients, 6 months—10 patients with one excluded, 8 months—10 patients with one excluded, and 18 months—30 patients with three excluded) completed FD-OCT follow-up.

Every single strut in the cross section evaluated was analyzed for distance from its luminal surface to the lumen contour, definition of malapposed struts was provided during the post-processing phase based on the knowledge of the total strut thickness (strut+polymer+drug), and a qualitative call was used for protruding and LIT struts.

Statistical analysis was done of the data collected in this example. The continuous variables are presented as median and the difference among three time points was analyzed by Kruskal-Wallis test with post-hoc Mann-Whitney U tests with the Bonferroni correction.

The safety at 18 months was evaluated in terms of MACE (major adverse cardiac events), at least, defined as death, MI i.e. myocardial infarction (Q-wave myocardial infarction, or “QWMI” and non-Q-wave myocardial infarction or “NQWMI”) and TVR (target vessel revascularization). At 18 months, 100% of the patients were evaluated for MACE. Table 14 shows the Events (as adjudicated by CEC) at 18 months, showing safety of the device. In summary, the MACE rate was 3.35 with no TLR (target lesion revascularization), TVR (target vessel revascularization) or ST (stent thrombosis) at 18 months. This data for MACE was not changed from that found at 8 months (See Example 2) or 12 months (See Example 3).

TABLE 14

| Events | Devices of Example 3 |
|---|---|
| as adjudicated by CEC | (N = 30 pts) |
| Total MACE* | 3.3% (1)* |
| Death | 0% |
| Q and non Q wave MI | 3.3% (1) |
| TVR | 0% |
| Peri-procedural MI | 6.7% (2)** |
| Stent Thrombosis | 0% |

*non TV, NQWMI: increased troponin following diagnostic angiogram at 44 days post-index procedure
**ppmi only: slight CK-MB elevation post-procedure, do not meet definition of non Q-wave MI

Angiography results (QCA) depicting the primary endpoint of in-stent late lumen loss is shown in Table 15 (also expressed in Table 4 in Example 2 and Table 13 in Example 3). The data revealed minimal change over time in late lumen loss from 4, 6, and 8 months to 18 months.

TABLE 15

| In-Stent LLL | 4-Month Group | 6-Month Group | 8-Month Group | 18-Month Group |
|---|---|---|---|---|
| (mm) | (n = 10) | (n = 10) | (n = 0) | (n = 25) |
| Mean (±SD) | 0.01 (±0.12) | 0.21* (±0.36) | 0.09 (±0.10) | 0.09 (±0.15) |
| Median | 0.03 (−0.22 to 0.21) | 0.10 (−0.03 to 1.20) | 0.08 (−0.01 to 0.28) | 0.08 (−0.07 to 0.46) |
| (Range) | | | | |

*Includes outlier with lesion outside proximal end to stent that grew into the stent. Excluding outlier the mean 6-mo LLL was 0.1 mm

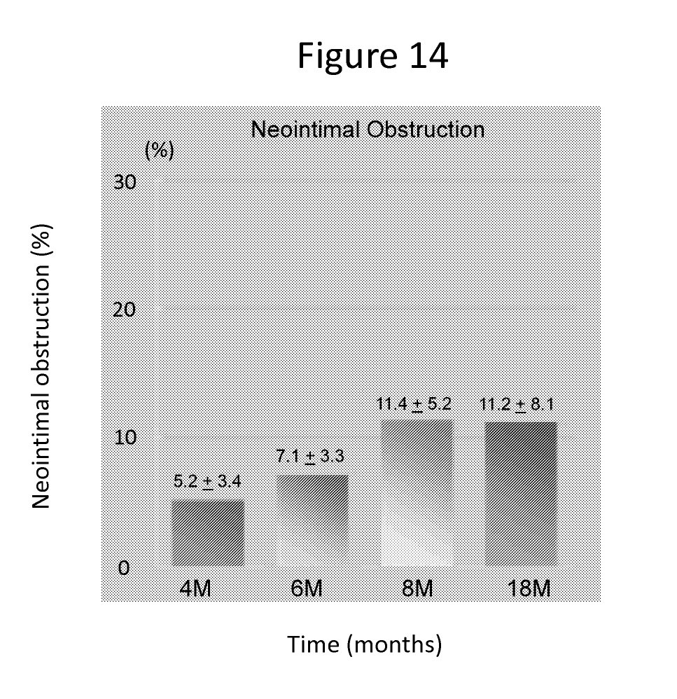

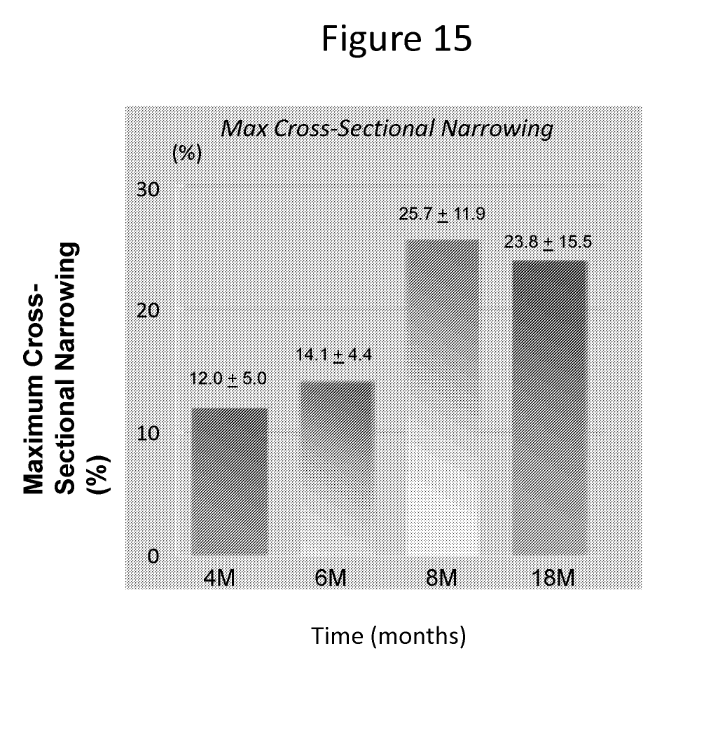

Table 16 shows the IVUS (3-D) results for each of the 4 month, 6 month, 8, and 18 month groups with respect to neointimal obstruction (%) and maximum cross sectional narrowing (%). The entire stent and adjacent reference segment up to 5 mm were analyzed by IVUS. The average percent neointimal obstruction at was 5.2%+/−3.4%, 7.1%+/−3.3%, 11.4%+/−5.2%, and 11.2%+/−8.1% at 4, 6, 8 and 18 months, respectively (FIG. 14). The average percent of maximum cross sectional narrowing was detected and determined by IVUS at 4 months to be 12.0%+/−5.0%, 14.1%+/−4.4%, 25.7%+/−11.9%, and 23.8%+/−15.5% at 4, 6, 8 and 18 months, respectively (FIG. 15). IVUS results demonstrate low neointimal hyperplasia and no evidence of progression of LLL from 8 month to 18 month follow up. There was no case in which there was lumen encroachment (which would be an encroachment over 50%).

TABLE 16
IVUS results

| | | | 8-month | 18-month |
|---|---|---|---|---|
| | 4-month group | 6-month group | group | group |
| | Mean (SD) | Mean (SD) | Mean (SD) | Mean (SD) |
| Parameter | (n = 9) | (n = 7) | (n = 8) | (n = 25) |
| neointimal obstruction | 5.2 (+/−3.4) | 7.1 (+/−3.3) | 11.4 (+/−5.2) | 11.2 (+/−8.1) |
| (%) | | | | |
| maximum cross section | 12.0 (+/−5.0) | 14.1 (+/−4.4) | 25.7 (+/−11.9) | 23.8 (+/−15.5) |
| narrowing (%) | | | | |

### Example 5

A human clinical trial was performed as a prospective, two-arm study with 184 patients at 26 sites evaluating Sirolimus DES (alternatively called a test device herein), a device as described herein and according to methods described herein (test device), and Endeavor® Sprint (Medtronic), a currently marketed DES device, for preliminary safety and efficacy. Patients were randomized in a 2:1 ratio (Sirolimus DES: Endeavor® Sprint) for implantation of one of the devices. Patients with discrete de novo lesions with vessel diameter and lesion length suitable for a 2.5 to 3.5 mm and a maximum of a 9-30 mm stent in native coronary arteries were enrolled. The patient selection criteria included stable or unstable angina pectoris (Class I, II, III, or IV), documented ischemia, or documented silent ischemia. Patients could not have recent Q wave MI (<72 hrs) and no elevated cardiac biomarkers. The target lesions included planned single, de novo, types A, B1 or B2 coronary lesions (according to the ACC/AHA classification) in the native coronary artery with >50% diameter stenosis. Vessel diameters were to be 2.5 to 3.5 mm and a maximum of a 9-30 mm long stent was used and indicated. Lesions were to be excluded if they were highly calcified, tortuous, thrombus present, or proximal angulation. Lesions were to be excluded if they were located at size branch over 2.5 mm, at an ostial location, or at a previously treated vessel. Non-target lesions were allowed to be treated if critical in another vessel prior to treating the target lesion.

Table 17 shows the Clinical and Lesion Characteristics of patients in this study.

TABLE 17
Baseline Clinical & Lesion Characteristics

| | | Test device | Endeavor ® | |
|---|---|---|---|---|
| Clinical Characteristics | | (n = 121) | Sprint (n = 60) | p-value |
| Demographics | Age (mean, range) | 64.9, +/−10.5 | 65.0, +/−10.5 | 0.956 |
| | Gender (m/f) | 69.4%/30.6% | 75.0%/25.0% | 0.488 |
| Risk Factors | MI | 23.5% | 16.7% | 0.337 |
| | PCI | 3.3% | 5.0% | 0.687 |
| | CABG | 4.2% | 3.3% | 1.000 |
| | Hypertension | 70.8% | 68.3% | 0.733 |
| | Hypercholesterolemia | 73.1% | 83.1% | 0.189 |
| | Diabetes | 18.5% | 20.0% | 0.841 |
| | Current smoker | 21.8% | 25.4% | 0.696 |
| Angina Status | Stable | 78.5% | 79.7% | 0.973 |
| | Unstable | 14.9% | 13.6% | |
| | Silent Ischemia | 6.6% | 6.8% | |
| | | Test device | Endeavor ® Sprint | |
| Lesion Characteristics | | (lesions = 121) | (lesions = 60) | p-value |
| Baseline | Length <10 mm | 27.3% | 31.7% | 0.843 |
| Angiography | Length 10-20 mm | 64.5% | 56.7% | |
| | Length >20 mm | 8.3% | 11.7% | |
| Lesion Location | Proximal | 35.5% | 46.7% | 0.478 |
| | Mid | 47.9% | 38.3% | |
| | Distal | 15.7% | 13.3% | |
| Vessel Location | RCA | 34.7% | 25.0% | 0.053 |
| | LAD | 43.8% | 36.7% | |
| | LCX | 21.5% | 38.3% | |
| Lesion | A | 8.3% | 10.0% | 0.991 |
| Classiciation | B1 | 46.3% | 45.0% | |
| | B2/C | 45.5% | 45.0% | |
| Calcification | Moderate-Severe | 27.3% | 31.7% | 0.602 |
| TIMI Flow 3 | | 91.7% | 91.7% | 0.987 |

Following the placement of either the test device or Endeavor® Sprint device, patients were followed up by imaging (angiography, intravascular ultrasound evaluation (IVUS) and optical coherence tomography (OCT)) at 9-months to determine in-stent late lumen loss (LLL), vessel healing, and stent coverage. Clinical safety was conducted for all 184 patients at 9-months.

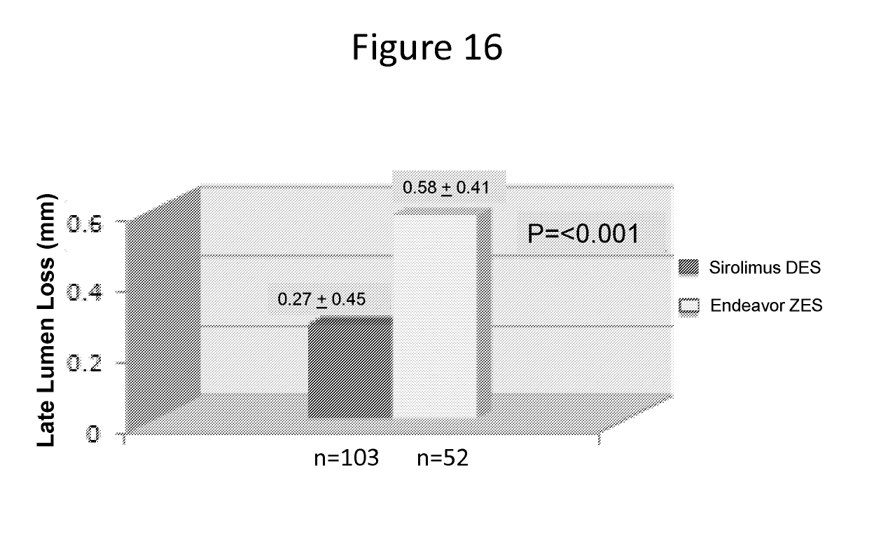

The test device patients evaluated at 9-months demonstrated an in-stent late lumen loss (LLL) of 0.27±0.46 mm, while the Endeavor® Sprint patients evaluated at 9-months demonstrated an in-stent late lumen loss (LLL) of 0.58±0.41 mm (p<0.001) (FIG. 16). Thus, the test device was superior to Endeavor® Sprint for the primary endpoint analysis of late lumen loss.

At procedure and at 9 months, OCT was conducted on a subgroup of 38 patients, 24 in the test device group and 14 in the Endeavor® Sprint group to determine the extent of stent strut coverage and strut malapposition. The OCT revealed a mean of 2% uncovered struts with good suppression of neointimal hyperplasia and minimal strut malapposition for the test device group (Table 18).

TABLE 18
Strut Coverage and Malapposition

| | Test device | Endeavor ® | |
|---|---|---|---|
| Measure | (n = 24) | Sprint (n = 14) | p-value |
| Strut Coverage - % | 2.05 +/− 3.99 | 0.52 +/− 1.46 | 0.048 |
| Uncovered Struts | | | |
| Max Segment Length | 0.88 +/− 1.09 | 0.53 +/− 1.31 | 0.072 |
| with Uncovered Strut (mm) | | | |
| % Strut Malapposition | 0.99 +/− 2.47 | 0.23 +/− 0.87 | 0.126 |
| Max Segment Length with | 0.44 +/− 0.095 | 0.17 +/− 0.64 | 0.148 |
| Malapposed Struts (mm) | | | |

Quantitative coronary angiography results obtained post procedure and at 9 months are represented in Table 19.

TABLE 19
Quantitative coronary angiography.

| | | | |
|---|---|---|---|
| Variable | Test device | Endeavor ® | |
| mean or % (n) | (n = 103) | Sprint (n = 52) | p-value |

Post-procedure

| | | | |
|---|---|---|---|
| In-stent MLD, mm | 2.83 ± 0.34 | 2,86 ± 0.38 | 0.69 |
| In-stent DS, % | 3.38 ± 7.29 | −0.72 ± 7.19 | <0.001 |
| In-lesion DS, % | 13.42 ± 7.61 | 14.13 ± 6.84 | 0.54 |

Follow-up 9-months

| | | | |
|---|---|---|---|
| In-stent MLD, mm | 2.58 ± 0.53 | 2.28 ± 0.51 | 0.001 |
| In-stent DS, % | 12.11 ± 16.10 | 19.34 ± 15.62 | 0.008 |
| In-lesion late loss, mm | 0.19 ± 0.43 | 0.34 ± 0.36 | 0.029 |
| In-stent binary restenosis, n | 5 (4.9%) | 1 (1.9%) | 0.66 |
| (%) | | | |
| In-lesion binary restenosis, n | 5 (4.9%) | 2 (3.8%) | 1.00 |
| (%) | | | |

At 9 months, endothelial function testing (EFT) was conducted on a subgroup of 29 patients, 19 in the test device group and 10 in the Endeavor® Sprint group to evaluate endothelial function by rapid atrial pacing (Table 20). Vasomotor Response was categorized as follows: Responder—dilation of the proximal/distal reference vessel segment after pacing indicating normal endothelial function; Non-responder—no dilation or constriction of the proximal/distal reference vessel segment after pacing indicating abnormal endothelium throughout the vessel; Vasoconstrictor—constriction of the proximal/distal reference vessel segment after pacing indicating endothelial dysfunction. EFT demonstrated an overall return on endothelial function with no incidence of vasoconstriction in either group.

TABLE 20
endothelial function testing (EFT) - Vasomotor Response

| | Test device | Endeavor ® | |
|---|---|---|---|
| EFT Status | (n = 19) | Sprint (n = 10) | p-value |
| Responders | 100% (19/19) | 90% (9/10) | 0.34 |
| Non-Responders | 0% (0/19) | 10% (1/10) | 0.34 |
| Vasoconstrictors | 0% (0/19) | 0% (0/10) | 1.00 |

The safety evaluation was conducted by reviewing MACE (death, MI and TVR) for the test device group and the Endeavor® Sprint group at 9 months. The clinical follow-up was available for 177 patients and all results were adjudicated by an independent CEC. The test device group experienced a lower TLR and MACE compared to the Endeavor® Sprint group (0.9% TLR and 4.3% MACE vs. 1.7% TLR and 6.7% MACE) with no definite/probable ST (Table 21).

TABLE 21
9-month MACE

| n = 177 | Test device | Endeavor ® Sprint | p-value |
|---|---|---|---|
| MACE | 4.3% | 6.7% | 0.49 |
| Death | 0.9% | 1.7% | 1.00 |
| MI | 2.6% | 3.3% | 0.49 |
| Ischemic TVR | 0.9% | 1.7% | 0.49 |
| Ischemic TLR | 0.9% | 1.7% | 1.00 |
| Definite/Probable ST | 0% | 1.7% | 0.33 |
| TVF | 3.4% | 6.7% | 0.45 |

As used herein, the term “about,” unless otherwise defined for the aspect to which it refers, means variations of any of 0.01%, 0.05%, 0.1%, 0.5%, 1%, 2%, 5%, 10%, 15%, 20%, 25%, 30%, and 50% on either side of the aspect target or on a single side of the aspect target, depending on the embodiment. When referring to an aspect that is expressed as a percent, the term about does not generally refer to a percent of the percent, but rather a range about the percent—unless otherwise stated. For non-limiting example, if an aspect was “about 5.0%” and the variation for about was 0.5% (depending on the embodiment), this could mean 5.0% plus or minus 0.5%—equating to a range of 4.5% to 5.5%.

The foregoing is illustrative of the present invention, and is not to be construed as limiting thereof. While embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method comprising providing a device comprising a stent and a coating on the stent wherein the coating comprises PLGA and crystalline sirolimus and wherein following implantation into a subject no more than 25% of the struts are malapposed at 4 months, no more than 20% of the struts are malapposed at 6 months, no more than 10% of the struts are malapposed at 8 months, and no more than 5% of the struts are malapposed at 18 months.

2. The method of claim 1, wherein at 8 months, no more than 5% of the struts are malapposed.

3. The method of claim 1, wherein a lot of devices which includes the device has an average percent malapposition of stent struts of about 1% at 4 months, about 1% at 6 months, about 0.5% at 8 months, and/or about 0.1% at 18 months.

4. The method of claim 1, wherein a lot of devices which includes the device has an average percent malapposition of stent struts of less than 1% at 4 months, less than 1% at 6 months, less than 0.5% at 8 months, and/or less than 0.1% at 18 months.

5. The method of claim 1, wherein a lot of devices which includes the device has an average percent of malapposed stent struts that trends downward over time.

6. The method of claim 1 wherein there is a homogeneous distribution of malapposed stent struts along the stent at any one or more of 4, 6, 8 and 18 months following implantation of the device in the subject.

7. The method of claim 1, wherein there is no abnormal intraluminal tissue related with any malapposed stent struts of the device.

8. A method comprising providing a device comprising a stent and a coating on the stent wherein the coating comprises PLGA and crystalline sirolimus and wherein following implantation into a subject there is no more than 50% of the struts are uncovered at 4 months, no more than 40% of the struts are uncovered at 6 months, no more than 15% of the struts are uncovered at 8 months, and no more than 10% of the struts are uncovered at 18 months.

9. The method of claim 8, wherein at 8 months, no more than 10% of the struts are uncovered.

10. The method of claim 8, wherein at 18 months, no more than 5% of the struts are uncovered.

11. The method of claim 8, wherein a lot of devices which includes the device has an average percent of uncovered stent struts of about 10% at 4 months, about 5% at 6 months, and/or about 5% at 8 months.

12. The method of claim 8, wherein a lot of devices which includes the device has an average percent of uncovered stent struts of about 10% at 4 months, about 5% at 6 months, about 5% at 8 months, and/or about 1% at 18 months.

13. The method of claim 8, wherein a lot of devices which includes the device has an average percent of uncovered stent struts of less than 11% at 4 months, less than 5% at 6 months, and/or less than 5% at 8 months.

14. The method of claim 8, wherein a lot of devices which includes the device has an average percent of uncovered stent struts of less than 11% at 4 months, less than 5% at 6 months, less than 5% at 8 months and/or less than 1% at 18 months

15. The method of claim 8, wherein a lot of devices which includes the device has an average percent of uncovered stent struts that trends downward over time.

16. The method of claim 8, wherein there is a homogeneous distribution of uncovered stent struts along the stent at any one or more of 4, 6, and 8 months following implantation of the device in the subject.

17. The method of claim 8, wherein there is no abnormal intraluminal tissue related with any uncovered stent struts of the device.

18. A method comprising providing a device comprising a stent and a coating on the stent wherein the coating comprises PLGA and crystalline sirolimus and wherein following implantation into a subject at least 0.02 mm of neointimal thickness exists on the stent on average at 4 months, at least 0.02 mm of neointimal thickness exists on the stent on average at 6 months, at least 0.06 mm of neointimal thickness exists on the stent on average at 8 months.

19. The method of claim 18, wherein a lot of devices which includes the device has an average neointimal thickness of about 0.07 mm at 4 months, about 0.1 mm at 6 months, and/or about 0.14 mm at 8 months.

20. The method of claim 18, wherein a lot of devices which includes the device has an average neointimal thickness of at least 0.05 mm at 4 months, about 0.075 mm at 6 months, and/or about 0.1 mm at 8 months.

21. The method of claim 18, wherein a lot of devices which includes the device has an average neointimal thickness that trends upward over time.

22. The method of claim 21, wherein there is a statistically significant difference in the neointimal thickness between the 4 month and the 8 month time point with respect to average neointimal thickness for the lot.

23. The method of any one of claim 1, 8, or 18, wherein: at most 10% of the stent struts are covered by fibrin at 4 months following implantation, at most 4% of the stent struts are covered by fibrin at 6 months following implantation, or at most 1% of the stent struts are covered by fibrin at 8 months following implantation.

24. The method of any one of claim 1, 8, or 18, wherein a MACE rate of a lot of devices which includes the device is at most 5%, between 1% and 5%, less than 5%, less than 4%, between 3% and 5%, or less than 3.5%.

25. The method claim 24 wherein: there is no target vessel revascularization and/or there is no stent thrombosis from implantation until 12 months following implantion.

26. A method comprising providing a device comprising a stent and a coating on the stent wherein the coating comprises PLGA and crystalline sirolimus and wherein following implantation into a subject the late lumen loss is less than 0.55 mm, less than 0.50 mm, less than 0.45 mm, less than 0.40 mm, less than 0.35 mm, or less than 0.30 mm at 9 months following implantation.

27. A method comprising providing a device comprising a stent and a coating on the stent wherein the coating comprises PLGA and crystalline sirolimus and wherein following implantation into a subject the target lesion revascularization (TLR) is less than 1.5%, or less than 1.0% at 9 months following implantation.

28. A method comprising providing a device comprising a stent and a coating on the stent wherein the coating comprises PLGA and crystalline sirolimus and wherein following implantation into a subject the MACE rate is less than 6.5%, less than 6.0%, less than 5.5%, less than 5.0%, less than 4.5%, or less than 4.0% at 9 months following implantation.

29. A method comprising providing a device comprising a stent and a coating on the stent wherein the coating comprises PLGA and crystalline sirolimus and wherein following implantation into a subject endothelial function is maintained with no incidence of vasoconstriction at 9 months following implantation.

30. A device provided according to any one of claims 1-29.
